(19) United States
(12) Patent Application Publication
(10) Pub. No.: US 2024/0156937 A1
(43) Pub. Date: May 16, 2024

(54) BACERIOPHAGE VIRUS-LIKE PARTICLES VACCINES FOR CHLAMYDIA TRACHOMATIS INFECTIONS

(71) Applicant: UNM RAINFOREST INNOVATIONS, Albuquerque, NM (US)

(72) Inventors: AMANDA L. COLLAR, ALBUQUERQUE, NM (US); KATHRYN MARIE FRIETZE, ALBUQUERQUE, NM (US)

(21) Appl. No.: 18/278,118

(22) PCT Filed: Apr. 20, 2022
(86) PCT No.: PCT/US2022/025542

§ 371 Date: Aug. 21, 2023

Related U.S. Application Data
(60) Provisional application No. 63/178,967, filed on Apr. 23, 2021.

(51) Int. Cl. A61K 39/118; A61K 47/69; A61P 37/04; C07K 14/005; C07K 14/295; C12N 7/00; A61K 39/00

(52) U.S. Cl. CPC A61K 39/118; A61K 47/6901; A61P 37/04; C07K 14/005; C07K 14/295; C12N 7/00; A61K 2039/5258; A61K 2039/575; C12N 2795/18122; C12N 2795/18123; C12N 2795/18134; C12N 2795/18171

(57) ABSTRACT

The present invention is directed virus-like particles (VLPs) which are useful in immunogenic compositions and vaccines epitopes mediating protection are disclosed. Immunogenic peptides are identified and are displayed on virus-like particles, especially Qbeta, MS2, or AP205 VLPs which provide a potent immunogenic response in a patient or subject and enhanced protection from Ct infection in a patient or subject. Pharmaceutical compositions and vaccines are disclosed as are methods for providing an immunogenic response and/or vaccinating a patient or subject against Chlamydia trachomatis infections.

### RELATED APPLICATIONS AND GRANT SUPPORT

This application claims the benefit of priority of provisional application Ser. No. US63/178,967, filed 23 Apr. 2021, the entire contents of which is incorporated by referenced herein.

This invention was made with government support under grant nos. KL2 TR001448, F30 AI156995, U19 AI113187 and UL1 TR001449 awarded by the National Institutes of Health (NIH). The government has certain rights in the invention.

### FIELD OF THE INVENTION

The present invention is directed virus-like particles which are useful in immunogenic compositions and vaccines for the prevention and treatment of Chlamydia trachomatis (Ct) infections. Methods of identifying Ct antibody epitopes mediating protection are disclosed. Immunogenic peptides, including peptides derived from Ct adhesion factors Outer membrane complex B (OmcB, CTT443), Major Outer Membrane Protein (MOMP, CTH522), Putative Outer Membrane Protein C (PmpC, CT414), Putative Outer Membrane Protein D (PmpD, CT812), and Pgp3 are identified and are displayed on virus-like particles, especially Qbeta, MS2, or AP205 VLPs which provide a potent immunogenic response in a patient or subject and enhanced protection from Ct infection in a patient or subject. Pharmaceutical compositions and vaccines are disclosed as are methods for providing an immunogenic response and/or vaccinating a patient or subject against Chlamydia trachomatis infections.

### BACKGROUND OF THE INVENTION

Chlamydia trachomatis (Ct) is the cause of the most common form of bacterial sexually transmitted infection (STI), causing long-term medical complications in a subset of women. Despite screening, the prevalence of these infections has increased dramatically and some 7 out of 10 infections of women are asymptomatic. The direct medical costs of these infections surpasses $500 million annually in the United States. Due to high prevalence, associated morbidity, and the relative ineffectiveness and high cost of screening programs, an effective vaccine for Ct would meet this growing need. With over 70 years of vaccine research being directed to finding a vaccine for Ct, developing an effective Ct vaccine would meet a long felt need. Studies have shown that antibodies may play a role in Ct infection.

### Brief Description of the Invention

Bacteriophage virus-like particles (VLPs) are highly immunogenic vaccine platforms that are multivalent platforms that can be used to dramatically increase the immunogenicity of molecules that are immunogenic and might provide an approach to vaccine development and use. The present invention is directed to virus-like particles (VLPs) derived from bacteriophages, especially Qbeta, MS2, or AP205 bacteriophage which are engineered to provide VLPs which are conjugated to immunogenic peptides or peptide insertions for providing immunogenic VLP compositions and vaccines for Chlamydia trachomatis (Ct). The immunogenic peptides are chemically conjugated at high density to the virus-like particles. The immunogenic peptides are conjugated principally to lysine residues which exist on the surface of the VLP and crosslinked to the immunogenic peptides for display at the surface of the VLP particles. In embodiments, the present invention addresses the failings of prior art attempts to provide vaccines to effectively prevent and/or reduce the likelihood of Ct infections and/or ameliorate the symptoms of Ct. In embodiments, VLP compositions and vaccines according to the present invention provide binding to multiple serovars of Ct and in embodiments provide multi-serovar and pan-serovar protection with binding to all clinically relevant Ct serovars and in certain embodiments, to all known Ct serovars. For example, VLP compositions and vaccines according to the present invention provide binding to multiple serovars of Ct such as ocular serovars (A-C), urogenital serovars (D-K) and lymphogranuloma serovars (LI, L2, L3). Although the present invention focuses principally on the urogenital serovars (D-K) and binds to multiple serovars and in preferred embodiments all of the urogenital serovars within this group , the VLPs and vaccines of the present invention also bind to multiple serovars such as the ocular serovars and lymphogranuloma serovars identified above.

The present invention meets a long-felt need in the art, which has searched for an approach to preventing and/or inhibiting Ct infection for more than years.

The present invention thus provides immunotherapeutic and prophylactic Qbeta bacteriophage viral-like particles (VLPs) conjugated to immunogenic peptides which are useful in the prevention of Chlamydia trachomatis (Ct) infection and medical consequences which occur from such an infection. Related compositions (e.g. immunogenic compositions, including vaccines) and therapeutic methods are also provided. VLPs and related compositions of the invention induce high-titer antibody responses to protect against Chlamydia trachomatis (Ct) infection, long term medical sequalae (such as pelvic inflammatory disease, upper genital tract inflammation and scarring, ectopic pregnancy, and infertility), and associated symptoms (such as discharge and/or bleeding from the vagina, penis or rectum of a subject, painful urination, painful sexual intercourse, bleeding between periods and/or after sexual intercourse in women and testicular pain in men) in subjects in need. VLPs, VLP-containing compositions, and therapeutic methods of the invention induce an immunogenic response against Chlamydia trachomatis (Ct) infection, confer immunity against, protect against and reduce the likelihood of such infections and related symptoms and morbidity as disclosed herein.

Accordingly, VLPs can be used as a platform to elicit rapid, high-titer, and long-lasting antibody responses to Chlamydia trachomatis (Ct) infection. These features are required for effective vaccine-based treatment for Chlamydia trachomatis (Ct) infection. In embodiments, the present vaccines provide an unexpectedly quick immunogenic response, which is a strong and long lasting response which represents an unexpected result.

In an embodiment, the present invention is directed to a composition comprising: (a) a virus-like particle (VLP) comprising a bacteriophage coat protein, often a Q-beta (Qβ bactiophage coat protein ; and (b) at least one conjugated peptide determinant; wherein said peptide determinant is displayed on said virus-like particle, and wherein said determinant comprises a conjugated immunogenic peptide of at least five contiguous amino acids of a peptide sequence according to the peptides of SEQIDNOS 1-75 hereof or as otherwise described herein. Immunopeptides which are conjugated to VLPs according to the present invention include the following:

| Immunogenic Peptide Sequence | SEQ ID NO: |
|---|---|
| FEGNSANFANGGAIAASGKVLFVAN | SEQ ID NO: 1 |
| GAQLTQTTSGSS | SEQ ID NO: 2 |
| ARAPQAIPTRSSD | SEQ ID NO: 3 |
| DLEDSVNSEK | SEQ ID NO: 4 |
| DGYKGAYGGASA | SEQ ID NO: 5 |
| TFFLEEEKLPSEAF | SEQ ID NO: 6 |
| SEQENNAEIG | SEQ ID NO: 7 |
| ALFASEDGDLSPESS | SEQ ID NO: 8 |
| STPVQQGHAISK | SEQ ID NO: 9 |
| QGHAISKPEAEIE | SEQ ID NO: 10 |
| SKPEAEIESSSEP | SEQ ID NO: 11 |
| ARAPQALPTQEEF | SEQ ID NO: 12 |
| KKEGRPLSSGYS | SEQ ID NO: 13 |
| ASEDGDLSPE | SEQ ID NO: 14 |
| GGAICTQNLTISQNTGNVLFYNN | SEQ ID NO: 15 |
| FEGNSAQLANGGAIAASGKVLFVAN | SEQ ID NO: 16 |
| GGAICTQNLTISQNNGNVLFLNN | SEQ ID NO: 17 |
| DGYRGSYIGASA | SEQ ID NO: 18 |
| LEEEKLPSE | SEQ ID NO: 19 |
| FASEDGDLSPESSISSEE | SEQ ID NO: 20 |
| FLEEEKLPSEAFISAEE | SEQ ID NO: 21 |
| GSQGDTADT | SEQ ID NO: 22 |
| APSGDQS | SEQ ID NO: 23 |
| LEGSQGDTADTGT | SEQ ID NO: 24 |
| FDTTTLNPTIAGAGDVK | SEQ ID NO: 25 |
| VDITTLNPTIAGSGSVA | SEQ ID NO: 26 |
| LDVTTLNPTIAGKGAVV | SEQ ID NO: 27 |
| LDVTTLNPTIAGKGTVV | SEQ ID NO: 28 |
| VDITTLNPTIAGSGSVV | SEQ ID NO: 29 |
| EFTINKPKGYVGKEFPLD | SEQ ID NO: 30 |
| LDVTTWNPTIAGAGTIA | SEQ ID NO: 31 |
| LKMTTWNPTISGSGI | SEQ ID NO: 32 |
| EFTINKPKGYVGQEFPLN | SEQ ID NO: 33 |
| ARKNHSK | SEQ ID NO: 34 |
| NTSHKSKKARKNSHSKET | SEQ ID NO: 35 |
| ATGPKQDSSFGRMY | SEQ ID NO: 36 |
| TTSHQKDRKARKNHQNRT | SEQ ID NO: 37 |
| ARKNHQN | SEQ ID NO: 38 |
| AVEPRQDSCFGKMY | SEQ ID NO: 39 |
| KSTPVAAKMTAS | SEQ ID NO: 40 |
| TNTGLTPTT | SEQ ID NO: 41 |
| SNNSSTNA | SEQ ID NO: 42 |
| RTSITNTGLT | SEQ ID NO: 43 |
| STTPVAAKITAS | SEQ ID NO: 44 |
| TNSGSTPTT | SEQ ID NO: 45 |
| TNNAQANS | SEQ ID NO: 46 |
| KTCVTNSGST | SEQ ID NO: 47 |
| SNNPSTNA | SEQ ID NO: 48 |
| INTGLTPTT | SEQ ID NO: 49 |
| GDSKPYAISYGY | SEQ ID NO: 50 |
| GDTQPCAISYGY | SEQ ID NO: 51 |
| TSTPVAAKMTAS | SEQ ID NO: 52 |
| RTRIINTGLT | SEQ ID NO: 53 |
| VDITTLNPTIAGCGSVV | SEQ ID NO: 54 |
| FDVTTLNPTIAGAGDVK | SEQ ID NO: 55 |
| LDVTTLNPTIAGKGSVV | SEQ ID NO: 56 |
| FDTTTLNPTIAGAGEVK | SEQ ID NO: 57 |
| AVEPRQDSCFGKMY | SEQ ID NO: 58 |
| ATGPKQDSCFGRMYGGGC | SEQ ID NO: 59 |
| TTLNPTIAG | SEQ ID NO: 60 |
| EDEQIGARIV | SEQ ID NO: 61 |
| NIYESIGGSRTSGPEN | SEQ ID NO: 62 |
| LNPTIAG | SEQ ID NO: 63 |
| LNPTIAGA | SEQ ID NO: 64 |
| APSGDQS | SEQ ID NO: 65 |
| PSGDQSI | SEQ ID NO: 66 |
| GSQGDTADT | SEQ ID NO: 67 |
| SQGDTADTG | SEQ ID NO: 68 |
| ASSGAPSGDQSISANA | SEQ ID NO: 69 |
| TNLEGSQGDTADTGTGDVN | SEQ ID NO: 70 |
| EFTINKPKGYVGKEFPLD | SEQ ID NO: 71 |
| | (MOMP AA 233-250) |
| FDTTTLNPTIAGAGDV | SEQ ID NO: 72 |
| | (MOMP AA 313-329) |
| ATGPKQDSCFGRMY | SEQ ID NO: 73 |
| | (OcmB AA 314-329) |
| LEGSQGDTADTGT | SEQ ID NO: 74 |
| | (PmpC AA 647-659) |
| ASEDGDLSPE | SEQ ID NO: 75 |
| | (Pmpd AA 764-773) |

Preferred immunogenic peptides are derived from Chlamydia trachomatis (Ct) adhesion factors Outer membrane complex B (OmcB, CTT443) and Major Outer Membrane Protein (MOMP, CTH522).

In embodiments, the bacteriophage coat protein used to form the VLPs is a Qβ, MS2, or AP205 bacteriophage single coat protein, preferably a Qβ bacteriophage single coat protein. In embodiments, the single coat protein is presented as a dimer. In embodiments, often 90 single coat protein dimers or 180 bacteriophage single coat proteins self-assemble into a VLP onto the surface of which immunogenic peptides comprising small peptide determinants derived from Ct proteins are conjugated. These immunogenic peptides (also referred to as immunodominant epitopes) are often obtained from Ct antigens. In embodiments, the immunogenic peptides which are conjugated to the surface of the VLP comprise between 5 and 25 contiguous amino acids of a peptide of SEQIDNO:1-75 derived from a Ct antigen, in particular, a Ct adhesion factor such as Major Outer Membrane Protein (MOMP, CT681)) Outer membrane complex B (OmcB, CT443), Putative outer membrane protein C (PmpC, CT414) and Putative outer membrane protein D (PmpD, CT812), other Ct antigens also provide immunogenic peptides for use in the present invention. In embodiments, these immunogenic peptides are often obtained from immunogenic peptides of SEQIDNOs:25-29, 54-58 or 71-75.

Immunogenic peptides which are conjugated to the surface of the VLP according to the present invention are obtained from the immunogenic peptides of SEQIDNO:1-75 or as otherwise described herein and are between 5 and 25 contiguous amino acids, 5 and 24 contiguous amino acids, 5 and 23 contiguous amino acids, 5 and 22 contiguous amino acids, 5 and 21 contiguous amino acids, 5 and 20 contiguous amino acids, 5 and 19 contiguous amino acids, 5 and 18 contiguous amino acids, 5 and 17 contiguous amino acids, 5 and 16 contiguous amino acids, 5 and 15 contiguous amino acids, 5 and 14 contiguous amino acids, 5 and 13 contiguous amino acids, 5 and 12 contiguous amino acids, 6 contiguous amino acids, 7 contiguous amino acids, 8 contiguous amino acids, 9 contiguous amino acids, 10 contiguous amino acids and 11 contiguous amino acids in length.

In embodiments, the VLPs are conjugated to the Ct immunogenic peptides through linkers as described herein. These linkers often comprise a crosslinker as described herein and an oligopeptide linker covalently bonded thereto. The crosslinker and oligopeptide linker may be covalently bonded directly to each other, or optionally through a covalent connector, to form the linker as described herein. Often, the crosslinker is bonded directly to the oligopeptide linker. The crosslinker is often bonded to the surface of the VLP through a sidechain of an amino acid (often, the butyleneamine sidechain of surface lysine amino acid residues), either directly or through a covalent connector. The crosslinker may be bonded directly to the immunogenic peptide or often to the oligopeptide linker, which is bonded to the immunogenic peptide directly or through a covalent connector. The number of immunogenic peptides which are conjugated to each VLP ranges from less than 1 to more than about 180, 10 to 180, 50 to 180, often 90 to 180 or in certain cases more (e.g often between 90-720, or between 1 and 4 conjugates per coat protein in the VLP).

In embodiments, linkers often are used to conjugate immunogenic peptides to the surface of the VLPs, from nucleophilic or electrophilic sites on side chains of amino acids of the coat polypeptide of the VLP (e.g. butyleneamine sidechains of lysine residues, among others) to electrophilic or nucleophilic sites (often, the carboxy or amine terminus of the immunogenic peptide or an oligopeptide linker) on the carboxy or amine terminus of the immunogenic peptide which links the immunogenic peptide to the crosslinker or directly to the VLP. In embodiments, often the carboxy terminus of the immunogenic peptides are linked to an oligopeptide which often comprises between 2 and 15 neutral amino acids, often between 3 and 10 neutral amino acids and terminates in a cysteinyl group which is further bonded to the crosslinker which can be conjugated to the VLP. The oligopeptide linker may be bonded to the immunopeptide directly or through a covalent connector. Often the oligopeptide comprises a cysteinyl group and between 2 and 5, often 3 or 4 neutral (often glycine) amino acid residues.

In embodiments of the present invention, the oligopeptide linker of the immunogenic peptide conjugate is covalently bonded to an electrophilic or nucleophilic group of the crosslinker (often, a carbonyl group, a vinyl group, a sulfhydryl group, an amine or hydroxyl group) directly or optionally through a covalent connector. Often the oligopeptide linker contains a terminal cysteinyl group which can be used to covalently bind the oligopeptide to the crosslinker. The crosslinker is also bonded to the VLP either directly or through a covalent connector to a nucleophilic or electrophilic group on an amino acid sidechain on the surface of the VLP (often lysine). The oligopeptide may be prepared or modified to facilitate the binding of the oligopeptide and immunogenic peptide to the crosslinker directly (through cysteine or another amino acid containing a functional group) or through a covalent connector. The crosslinker is bonded to the nucleophilic or electrophilic amino acid residues, preferably lysine residues, on the surface of the VLP. The crosslinker may be optionally modified to promote covalent binding between the VLP and the crosslinker (which is linked through the oligopeptide linker to the immunogenic peptide). In embodiments, the oligopeptide of the linker is a 3 to 15 mer, often a 4 to 10 mer oligopeptide comprising neutral amino acid residues bonded to nucleophilic or electrophilic sites of the immunogenic peptide (which is often an amine group or carboxylic acid group of the immunogenic peptide). In embodiments, on one end of the oligopeptide, often the carboxyl terminus, the oligopeptide comprises a cysteinyl group or other amino acid which may be used to link the oligopeptide to the crosslinker. The amino end of the oligopeptide linker may optionally be conjugated to the immunogenic peptide through the use of a covalent connector such as a short amide linker (e.g. a C₁-C₄ alkyl amide group which forms a urea or urethane group with the peptide) or other group, among others. Often the oligopeptide linker is conjugated to the immunogenic peptide by forming a covalent bond directly with the amine terminus or carboxyl terminus of the immunogenic peptide, often the carboxy terminus.

In embodiments, the neutral amino acid residues of the oligopeptide are selected from the group consisting of glycine, alanine, valine, leucine, isoleucine, phenylalanine, tryptophan, methionine, proline, serine and mixtures thereof. In embodiments, the neutral amino acids often are selected from the group consisting of glycine, alanine, valine, leucine, isoleucine, serine and mixtures thereof, more often glycine or alanine, most often glycine. In embodiments, the oligopeptide is GGGC (SEQ ID NO: 76).

In embodiments, a covalent connector group is used to bridge the VLP, the crosslinker, the oligopeptide linker and/or the immunogenic peptide depending principally upon the functional groups on the amino acid residues of the VLP and the crosslinker. Often the amino acid residues on the VLP are lysine residues and the crosslinker may vary, but often is a (Succinimidyl 6-((beta-maleimidopropionamido)hexanoate)) (SMPH) crosslinker.

Common connector groups which are used in the present invention include the following chemical groups:

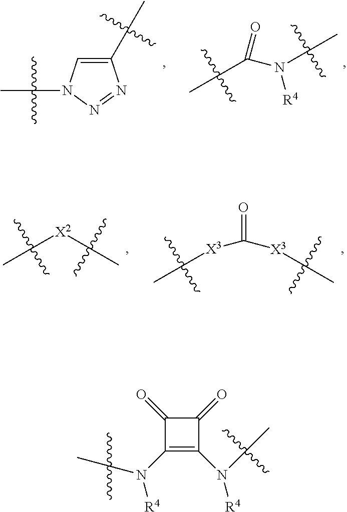

Where X² is O, S, NR 4 , C(O), S(O), S(O)₂, —S(O)₂O, —OS(O)₂, or OS(O)₂O;
X³ is O, S, NR₄; and
R⁴ is H, a C₁-C₃ alkyl or alkanol group, or a —C(O)(C₁-C₃) group. The amide group, indicated above, is a preferred connector group. In each connector group, the connector group may be extended with a C₁-C₄ alkylene group at a point of attachment to bind to a VLP, crosslinker, oligopeptide and/or immunogenic peptide (e.g, preferably between the amino acid residue on the VLP and the crosslinker, between the crosslinker and the oligopeptide of the linker or between the oligopeptide of the linker and the immunogenic peptide).

In embodiments, in the composition according to the present invention the immunogenic peptide conjugate is displayed at one or more nucleophilic or electrophilic amino acid residues on the surface of the VLP, often at a plurality of lysine residues on the surface of the VLP. In embodiments, the immunogenic peptide conjugate is displayed on the bacteriophage at the lysine residues by covalently binding an immunogenic peptide as described herein to the lysine residues through a crosslinker group and optional connector group. In embodiments, the linker group comprises a 3 to 15 mer, preferably a 4 to 10 mer oligopeptide linker covalently bonded to a crosslinker which is bonded to the VLP particle as described herein. Often the linker group comprises a cysteinyl group at the carboxyl end of the oligopeptide and 2 to 5 neutral amino acid residues, often 3 or 4 glycine amino acid residues.

In embodiments of the present invention, the oligopeptide linker of the immunogenic peptide conjugate is covalently bonded to an electrophilic or nucleophilic group of the crosslinker (e.g. a carbonyl group, a vinyl group, an amine or hydroxyl group) which optionally has been modified to facilitate the binding of the oligopeptide and immunogenic peptide to the crosslinker through a covalent connector and the crosslinker is bonded to the nucleophilic or electrophilic amino acid residues, preferably lysine residues on the surface of the bacteriophage through the crosslinker, which may be optionally modified with a covalent connector to promote covalent binding between the bacteriophage and the crosslinker (which is often linked through the oligopeptide linker to the immunogenic peptide). In embodiments, the oligopeptide of the linker is a 3 to 15 or 4 to 15 mer, preferably a 4 to 10 mer oligopeptide comprising neutral amino acid residues which are bonded to nucleophilic or electrophilic sites of the immunogenic peptide (which is often an amine group or carboxylic acid group of the immunogenic peptide).

In embodiments, on one end of the oligopeptide linker, often the carboxyl terminus, the oligopeptide linker comprises a cysteinyl group or other amino acid which may be used to link the oligopeptide linker to the crosslinker. The amino end of the oligopeptide may optionally be conjugated to the peptide through the use of a short amide linker (e.g. a C₁-C₄ alkyl amide group which forms a urea or urethane group with the peptide or other group, among others. At the other end of the oligopeptide linker, often the oligopeptide linker is conjugated to the immunogenic peptide by forming a covalent bond with the amine terminus or carboxyl terminus of the immunogenic peptide.

In embodiments, the neutral amino acid residues are selected from the group consisting of glycine, alanine, valine, leucine, isoleucine, phenylalanine, tryptophan, methione, proline, serine and mixtures thereof. In embodiments, the neutral amino acids often are selected from the group consisting of glycine, serine and mixtures thereof, more often glycine. In embodiments, the crosslinker is (Succinimidyl 6-((beta-maleimidopropionamido)hexanoate)) (SMPH) and the oligopeptide comprises three glycine amino acid residues and a cysteine amino acid residue at the carboxy end of the immunopeptide, wherein the sulfhydryl group of the cysteine residue binds to the crosslinker.

In embodiments, the immunogenic peptide comprises at least five contiguous amino acids of the immunogenic peptide sequence of an immunogenic peptide as described herein, often at least five contiguous amino acids from an immunogenic peptide of SEQIDNOs 1-75, often SEQIDNOs:1-10, 25-29, 54-58 or 71-75 hereof or as otherwise disclosed herein. In embodiments, the immunogenic peptide is a peptide according to the sequence of SEQ ID Nos: 25-29, 54-58 or 71-75 as described herein.

In embodiments, the present invention is directed to a population of virus-like particles (VLPs) derived from Qbeta or AP205 bacteriophages to which short chain immunopeptides derived from Chlamydia trachomatis (Ct) antigenic peptides such as Major outer membrane protein (MOMP), especially amino acids 233-250 and 314-329; Outer membrane complex B (OcmB), especially amino acids 86-99; Putative outer membrane protein C (PmpC), especially amino acids 647-659 and Putative outer membrane protein D (PmpD), especially amino acids 764-773 are conjugated to the VLPs as otherwise described herein. In preferred embodiments, an immunogenic peptide of SEQIDNO: 1-75, often an immunogenic peptide of SEQIDNO: 25-29, 54-58 or 71-75 is conjugated to the VLPs. In embodiments, the population of conjugated VLPs is combined with a pharmaceutical carrier, additive and/or excipient to provide immunogenic compositions according to the present invention. In embodiments the population of VLPs is formulated into a pharmaceutical composition as a vaccine formulation (vaccine) for immunizing a patient or subject against a Chlamydia trachomatis (Ct) infection. In embodiments, the pharmaceutical composition comprises an adjuvant or other active component to enhance or facilitate an immunogenic response in a patient or subject to which the composition is administered. In embodiments, the population of VLPs may include a mixture of VLPs each of which is conjugated to a different immunopeptide (or contains a genetic insertion) or more than one immunopeptide as described herein.

In embodiments, in the composition according to the present invention which comprises a population of VLPs, the immunogenic peptide is displayed at one or more nucleophilic or electrophilic amino acid residues on the surface of the VLP, preferably at a plurality of lysine residues (often the butyleneamine side chain of lysine) on the surface of the VLP. In embodiments, the immunogenic peptide is displayed on the bacteriophage at lysine residues on the coat polypeptide by covalently binding an immunogenic peptide of Ct or more often, SEQ ID NO:1-75 or SEQIDNO: 25-29, 54-58 or 71-75 to lysine residues of the coat polypeptide through a linker group which includes a crosslinker and an oligopeptide linker. In embodiments, the oligopeptide linker group comprises a 4 to 15 mer, preferably a 4 to 10 mer oligopeptide covalently bonded to a crosslinker as described herein.

In embodiments, the present invention is directed to a method for enhancing an immune response against a Chlamydia trachomatis (Ct) infection in a patient or subject in need comprising introducing a pharmaceutical composition comprising a population of VLPs as otherwise described herein to said subject or patient, wherein an enhanced immune response against said Ct infection is produced in said patient or subject. In embodiments, the present invention is directed to a method for reducing the likelihood of a Chlamydia trachomatis (Ct) infection and/or morbidity in a patient or subject in need. In embodiments, the present invention is directed to a method wherein the composition is prophylactic for a Chlamydia trachomatis (Ct) infection.

In embodiments, the present invention is directed to a method of inducing an immunogenic response in a patient or subject comprising administering a composition comprising an effective amount of a population of immunogenic peptide conjugated VLPs as otherwise described herein to said patient or subject.

In embodiments, the present invention is directed to a method for treating or inhibiting a Chlamydia trachomatis (Ct) infection, transmission, morbidity, or a symptom thereof in a patient or subject in need comprising administering to said patient or subject a composition comprising an effective amount of a population of immunogenic peptide conjugated VLPs as otherwise described herein to said patient or subject.

In embodiments, the present invention is directed to a method of identifying immunogenic peptides and/or epitopes which can be used to raise an immunogenic response to a Chlamydia trachomatis (Ct) infection in a patient or subject. These methods are disclosed in the detailed description of the invention and the examples, set forth herein.

In embodiments, the invention is directed to compositions comprising a population of VLPs conjugated to immunogenic peptides as described herein, optionally in combination with a pharmaceutically acceptable carrier, additive and/or excipient. In embodiments, vaccines according to the present invention comprise an amount of VLPs to which immunopeptides are conjugated effective to elicit an immunogenic response, often a protective effect (which effect may include an attenuated infection) against Ct infection in the patient or subject administered said vaccines.

In embodiments, the immunogenic peptide is conjugated to the surface of the VLP at high density. In embodiments, the immunogenic peptide conjugate determinant is displayed at one or more lysine residues on the surface of the VLP which comprises a bacteriophage coat protein, especially a Qβ or AP205 bacteriophage coat protein. In embodiments, the immunogenic peptide determinant is displayed at the A-B loop, N-terminus or carboxy terminus of the bacteriophage coat protein.

In embodiments, the bacteriophage coat protein used to form the VLPs is a coat protein derived from Qbeta or AP205 bacteriophage, preferably a coat protein derived from Qbeta bacteriophage, often a dimer coat protein.

In embodiments, in the composition according to the present invention the immunogenic peptide conjugate determinant is displayed at one or more nucleophilic or electrophilic amino acid residues on the surface of the bacteriophage, preferably at a plurality of lysine residues on the surface of the VLP. In embodiments, the immunogenic peptide conjugate is displayed on the bacteriophage at the lysine residues by covalently binding an immunogenic peptide as described above and/or in attached examples to the lysine residues through a linker group. In embodiments, the linker group comprises a 4 to 15 mer, preferably a 4 to 10 mer oligopeptide covalently bonded to a crosslinker which is bonded to the VLP particle as described herein and as presented herein below.

In embodiments of the present invention, the oligopeptide linker of the immunogenic peptide conjugate is covalently bonded to an electrophilic or nucleophilic group of the crosslinker (e.g. a carbonyl group, a vinyl group, an amine or hydroxyl group) which optionally has been modified to facilitate the binding of the oligopeptide and immunogenic peptide to the crosslinker and the crosslinker is bonded to the nucleophilic or electrophilic amino acid residues, preferably lysine residues on the surface of the bacteriophage through the crosslinker, which may be optionally modified to promote covalent binding between the bacteriophage and the crosslinker (which is linked through the oligopeptide linker to the immunogenic peptide). In embodiments, the oligopeptide of the linker is a 4 to 15 mer, preferably a 4 to 10 mer oligopeptide comprising neutral amino acid residues bonded to nucleophilic or electrophilic sites of the immunogenic peptide (which is often an amine group or carboxylic acid group of the immunogenic peptide). In embodiments, on one end of the oligopeptide, often the carboxyl terminus, the oligopeptide comprises a cysteinyl group or other amino acid which may be used to link the oligopeptide to the crosslinker. The amino end of the oligopeptide may optionally be conjugated to the peptide through the use of a short amide linker (e.g. C₁-C₄ alkyl amide group which forms a urea or urethane group with the peptide or other group, among others. At the other end of the oligopeptide linker, often the oligopeptide linker is conjugated to the immunogenic peptide by forming a covalent bond with the amine terminus or carboxyl terminus of the immunogenic peptide.

In embodiments, the neutral amino acid residues are selected from the group consisting of glycine, alanine, valine, leucine, isoleucine, phenylalanine, tryptophan, methione, proline, serine and mixtures thereof. Embodiments, the neutral amino acids often are selected from the group consisting of glycine, serine and mixtures thereof, more often glycine.

In embodiments, the immunogenic peptide comprises at least five contiguous amino acids of the immunogenic peptide sequence of SEQ ID NOs. 1-75 hereof or as otherwise disclosed herein.

In embodiments, the present invention is directed to a population of virus-like particles as otherwise described herein.

In embodiments, the present invention is directed to a pharmaceutical composition comprising a population of virus-like particles as described herein in combination with a pharmaceutically acceptable carrier, additive and/or excipient, or alone. In embodiments, the composition is formulated for administration to a subject or patient as a vaccine. In embodiments the pharmaceutical composition or vaccine comprises an adjuvant (e.g., Advax, MF 59, CPG 1018, AS01B, AS03, AS04, etc.).

In embodiments, the present invention is directed to a method for enhancing an immune response against a Chlamydia trachomatis (Ct) infection in a patient or subject in need comprising introducing a pharmaceutical composition comprising a population of VLPs as otherwise described herein to said subject or patient, wherein an enhanced immune response against said Chlamydia trachomatis (Ct) infection is produced in said patient or subject. In embodiments, the present invention is directed to a method for reducing the likelihood of a Chlamydia trachomatis (Ct) infection and/or morbidity in a patient or subject in need. In embodiments, the present invention is directed to a method wherein the composition is prophylactic for a Chlamydia trachomatis (Ct) infection.

In embodiments, the present invention is direct to a method of inducing an immunogenic response in a patient or subject comprising administering a composition comprising an effective amount of a population of immunogenic peptide VLPs as otherwise described herein to said patient or subject.

In embodiments, the present invention is directed to a method for treating or inhibiting a Chlamydia trachomatis (Ct) infection, morbidity, or a symptom thereof in a patient or subject in need comprising administering to said patient or subject a composition comprising an effective amount of a population of immunogenic peptide conjugated VLPs as otherwise described herein to said patient or subject.

In embodiments, the present invention is directed to a method of identifying immunogenic peptides and/or epitopes which can be used to raise an immunogenic response to a Chlamydia trachomatis (Ct) infection in a patient or subject. These methods are disclosed herein and further presented in the describe experimental section.

In embodiments, the infection is a Chlamydia trachomatis (Ct) infection and the symptom is a symptom associated with said infection, such as discharge and/or bleeding from the vagina, penis or rectum of a subject, painful urination, painful sexual intercourse, bleeding between periods and/or after sexual intercourse in women and testicular pain in men. The morbidity is a morbidity associated with said infection, such as pelvic inflammatory disease, ectopic pregnancy, and tubal factor infertility caused by fallopian tube scarring.

In embodiments, the present invention is directed to a method for treating or reducing the likelihood of a Chlamydia trachomatis (Ct) infection, morbidity, or a symptom thereof in a patient or subject in need comprising administering to said patient a composition comprising an effective amount of a population of immunogenic peptide conjugated VLPs as otherwise described herein to said patient or subject. In embodiments, the symptom is one or more of discharge and/or bleeding from the vagina, penis or rectum of a subject, painful urination, painful sexual intercourse, bleeding between periods and/or after sexual intercourse in women and testicular pain in men. In embodiments, the morbidity is one or more of pelvic inflammatory disease, ectopic pregnancy, fallopian tube scarring, and tubal factor infertility.

The present invention is therefore directed to vaccines which target Chlamydia trachomatis (Ct) infection or a symptom or morbidity thereof in a patient or subject in need as otherwise disclosed herein for prophylactic and/or therapeutic purposes.

### BRIEF DESCRIPTION OF THE FIGURES

FIG. 1 shows the engineering bacteriophage Qβ virus-like particle-based vaccines targeting the variable domain 4 of the major outer membrane protein. A) Amino acid sequences of the core conserved VD4 epitope and flanking regions for all urogenital Ct serovars and murine Cm. B) Peptide ELISA demonstrating binding potential of the E4 monoclonal to a peptide containing flanking regions of the VD4 epitope (CtsvDE, FDTTTLNPTIAGAGDVK) and to a peptide containing only the conserved core epitope (CtVD4CONS, LNPTIAG). C) 180 Qβ coat proteins self-assemble into a Qβ VLP, which contains surface-exposed lysines (yellow). Using a bifunctional crosslinker, SMPH, modified synthetic peptides of the VD4 epitope containing a terminal tri-glycine linker sequence (blue) and a cysteine (red) can be chemically conjugated to surface-exposed lysines of the Qβ VLP. D) Denaturing SDS-PAGE gel confirms successful conjugation of each individual peptide to the surface of the coat protein, as noted by increased molecular weight of the coat protein.

FIG. 2 shows the immunogenicity of Qβ-VD4 vaccination. A) Schematic of immunization schedule in female NZW rabbits. Rabbits received 2-3 subcutaneous doses 3-weeks apart before terminal sera collection. B) Peptide ELISAs demonstrating binding potential of immune sera IgG to each cognate peptide included in the Qβ-Ct VD4 vaccination (panels 1-5) and Qβ-Cm VD4 vaccination (panel 6), with comparison between antibody-titers after the first (open circles) and terminal dose (closed circles). Unconjugated Qβ immune sera IgG binding utilized as a negative control (squares).

FIG. 3 shows the binding capacity of immune sera to Ct elementary bodies. A) CtsvD EB ELISA demonstrating Qβ-Ct VD4 binding capacity across reciprocal serum dilutions, as compared to Qβ control immune serum. B) EB ELISA demonstrating Qβ-Ct VD4 binding capacity to remaining urogenital Ct serovars and murine Cm, 1:32 serum dilution. C) Amino acid sequences of the core conserved VD4 epitope and flanking regions for ocular trachoma causing Ct serovars (A-C) and lymphogranuloma venereum causing Ct serovars (L1-3), as compared to CtsvD. D) EB ELISA demonstrating Qβ-Ct VD4 binding capacity to ocular trachoma and LGV causing Ct serovars, 1:32 serum dilution. All data shown are technical replicates. Statistical analysis was performed utilizing nonparametric Mann-Whitney t-test. Quantitative data represents the mean±SEM.

FIG. 4 shows intrauterine CtsvD murine challenge. A) Schematic of immunization schedule in female C57BL/6 mice. Mice received 3 intramuscular doses 3-weeks apart. Medroxyprogesterone acetate was administered subcutaneously at days 10 and 3 pre-infection. Mice were challenged with 10⁵ IFU via intrauterine instillation. Primary outcomes for mice were either determination of bacterial burden at day 3 post-infection or determination of upper genital tract pathology at day 60 post-infection. B) Peptide ELISA to determine antibody titers of both bacterial burden and pathology mouse groups before challenge at day 64. Quantitative data represents the mean±SEM. C) EB ELISA to demonstrate binding of pooled murine sera (day 64) to CtsvD, 1:32 serum dilution. D) Day 3 post-infection bacterial burdens, measured by qPCR of upper genital tract homogenates (pg of 16S/μg of GAPDH). E) Representative pathology of excised upper genital tracts; gross (upper), fixed (middle), sectioned and H&E stained (bottom). F) Uterine horn dilation scores of both vaccinated and control mice. Quantitative data represents the median.

FIG. 5 shows luciferase-expressing Cm murine challenge. A) Schematic of immunization schedule in female Balb/c mice. Mice received 3 intramuscular doses 3-weeks apart. Medroxyprogesterone acetate was administered subcutaneously at day 7 pre-infection. Mice were challenged with 2×10⁴ IFU of Luc-Cm via vaginal instillation. Infection was monitored via IVIS days 3 through 6 post-infection. B-C) Average radiance (p/s/cm²/sr) measured for the Qβ-Cm VD4 vaccinated group and the unconjugated Qβ vaccinated control group on days 3 through 6 post-infection. D) Area under the curve (AUC) measured for bacterial burden (average radiance) on days 3 through 6 post-infection and percent reduction of area under the curve. E) IVIS images for Qβ control mice (top) and Qβ-Cm VD4 mice (bottom) on day 6 post-infection, with luminescence visualized. Statistical analysis was performed utilizing nonparametric Mann-Whitney t-test. Quantitative data represents the mean±SEM.

FIG. 6 shows in vivo neutralization to determine antibody-mediated protection. A) Schematic of in vivo neutralization schedule in female Balb/c mice. Two mice received 3 intramuscular doses 3-weeks apart, followed by terminal sera collection. Medroxyprogesterone acetate was administered to naive mice subcutaneously at day 7 pre-infection. Heat-inactivated pooled sera was allowed to incubate with Luc-Cm before vaginal instillation of 2×10⁴ IFU of sera-Luc-Cm via vaginal instillation. Infection was monitored via IVIS days 3 through 6 post-infection. B-C) Average radiance (p/s/cm²/sr) measured days 3 through 6 post-infection for naive mice receiving vaginal Qβ-Cm VD4 serum or Qβ control serum. D) Area under the curve (AUC) measured for bacterial burden (average radiance) on days 3 through 6 post-infection and percent reduction of area under the curve. E) IVIS images for naive mice receiving vaginal Qβ serum (top) and Qβ-Cm VD4 serum (bottom) on day 3 post-infection, with luminescence visualized. Statistical analysis was performed utilizing nonparametric Mann-Whitney t-test. Quantitative data represents the mean±SEM.

FIG. 7 shows the longevity of peripheral and mucosal murine antibody responses. A) Peptide ELISA measuring Qβ-Ct VD4 IgG peripheral serum end-point serum dilution antibody titer through week 45 to cognate CtsvDE VD4 peptide. B) Peptide ELISA measuring Qβ-Cm VD4 IgG peripheral serum end-point serum dilution antibody titer through week 45 to cognate Cm VD4 peptide. C) Peptide ELISA measuring Qβ-Ct VD4 IgG mucosal serum end-point serum dilution antibody titer through week 45 to CtsvDE VD4 peptide. D) Peptide ELISA measuring Qβ-Ct VD4 IgG mucosal end-point dilution antibody titer their respective VD4 peptides, measured 3-weeks post-3^{rd} dose. E) Peptide ELISA measuring Qβ-Cm VD4 IgG mucosal serum end-point serum dilution antibody titer through week 45 to Cm VD4 peptide. F) Peptide ELISA measuring Qβ-Ct VD4 and Qβ-Cm VD4 IgA mucosal end-point dilution antibody titer their respective VD4 peptides, measured 6-weeks post-3^{rd} dose. Arrows indicate immunization administration.

FIG. 8 shows the engineering of bacteriophage Qβ VLP-based vaccines targeting the immunodominant epitope of OmcB. (A) Amino acid homology among the human urogenital Chlamydia trachomatis serovars (D-K) and murine Chlamydia muridarum of the immunodominant B cell epitope of Ct adhesion factor, OmcB. (B) Qβ VLP self-assembles and is composed of 180 coat proteins. Short peptide epitopes can be chemically conjugated to surface-exposed lysines via the bifunctional crosslinker SMPH. Protein epitopes are modified to contain a terminal tri-glycine linker sequence (blue), followed by a terminal cysteine (orange) to facilitate conjugation. Diagram illustrates C-terminal conjugation of the Ct amino acid sequence.

FIG. 9 shows Qβ VLP-based vaccines targeting the immunodominant OmcB epitope are highly immunogenic. (A) Vaccine strategy, in which a female NZW rabbit was immunized twice with their respective vaccine (Qβ-Ct OmcB, Qβ-Cm OmcB, or unconjugated Qβ control). Terminal sera was collected to determine immunogenicity via in vitro assays. (B & C) Terminal serum cognate peptide-specific IgG was assessed by peptide ELISA for Qβ-Ct OmcB (B) and Qβ-Cm OmcB (C).

FIG. 10 shows the binding characteristics of Qβ-Ct OmcB and Qβ-Cm OmcB immune sera. (A & B) Elementary body ELISAs investigating antibody binding to CtsvD EBs (A, 1:4 sera dilution) and Cm EBs (B, 1:8 sera dilution). (V-Cm OmcB demonstrates cross-reactivity to CtsvD. (C) Cross-reactivity of Qβ-Cm OmcB immune sera toward CtsvD elementary bodies was assessed by ELISA where Qβ-Cm OmcB immune sera, Qβ-VD4 MOMP immune sera (positive control), and unconjugated Qβ immune sera (negative control) binding was measured across serum dilutions. (D) Peptide ELISA was used to assess cross-reactivity of Qβ-Ct OmcB immune sera to Cm OmcB peptide, and Qβ-Cm OmcB immune sera binding to Ct OmcB cognate peptide.

FIG. 11 shows that immunization with Qβ-Cm OmcB results in decreased bacterial burden. (A) Vaccine strategy, in which female Balb/c mice were immunized with their respective vaccine (n=10 β-Cm OmcB, n=8 Qβ) 3 times, 3 weeks apart. 7 days before vaginal challenge, mice were administered 2.5 mg of medroxyprogesterone acetate to prolong and synchronize estrous cycles. Mice were then challenged with 2×10⁴ IFU of luciferase-expressing Cm vaginally. Infection was monitored via IVIS at days 3-8 post-infection. (B) Bacterial burden was assessed via IVIS at days 3-8 post-infection, measured by average radiance (p/s/cm²/sr); time course visualized. (C & D) Calculated area under the curve, resulting in a 0.56 log mean reduction in bacterial (C) and percent reduction of AUC normalized to Qβ, resulting in 72.5% reduction (D). Statistical analysis was performed utilizing nonparametric Mann-Whitney t-test. Quantitative data represents the mean±SEM.

FIGURE S1 shows a comparison of Qβ-Ct VD4 sera IgG avidity indexes across serial dilutions between those collected 3-weeks after the first and terminal dose using VD4 CtsvDE peptide (A) and VD4 CtsvK peptide (B).

FIGURE S2 shows the pathology of excised upper genital tracts from female mice vaccinated with Qβ-VD4 (n=10); gross (upper), fixed (middle), sectioned and H&E stained (bottom).

FIGURE S3 shows the pathology of excised upper genital tracts from female mice vaccinated with unconjugated Qβ control (n=10); gross (upper), fixed (middle), sectioned and H&E stained (bottom).

FIGURE S4 shows A) Peptide ELISA to determine sera IgG antibody titers of Qβ-Cm VD4 vaccinated mice before Luc-Cm challenge, at day 78. B) Peptide ELISA to determine sera IgG antibody titers of terminal mouse sera utilized for in vivo neutralization assay. Quantitative data represents the mean±SEM.

FIGURE S5 shows peptide ELISA measuring murine Qβ-Ct VD4 IgG peripheral pooled serum end-point serum dilution antibody titer through week 45 to CtsvF, G, HIaJ, and K VD4 peptides, respectively, demonstrating binding potential to each cognate peptide included within the Qβ-Ct VD4 vaccine.

FIGURE S6 shows that immunization with Qβ-Cm OmcB elicits high titer antibodies prior to Cm-luciferase challenge study. Vaccine strategy, in which female Balb/c mice were immunized with their respective vaccine (n=10 Qβ-Cm OmcB, n=8 Qβ) 3 times, 3 weeks apart. 7 days before vaginal challenge, mice were administered 2.5 mg of medroxyprogesterone acetate to prolong and sync estrous cycles. Mice were then challenged with 2×104 IFU of luciferase expressing Cm vaginally. Infection was monitored via IVIS at days 3-8 post-infection. Sera from mice were collected 28 days post final immunization and assessed for IgG to cognate Cm-OmcB peptide by ELISA.

FIGURE S7 shows intravital imaging of mice challenged with Cm-luciferase. (A) Detectable bacterial burden measured by radiance (p/s/cm²/sr) for individual mice over time. Mice 1 and 9 (orange boxes) of the Qβ control group were excluded because of incorrect vaccination administration. (B) Calculated average radiance for each mouse is shown.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984,“Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal and preferably a human, to whom treatment, including prophylactic treatment (prophylaxis), with the immunogenic compositions and/or vaccines according to the present invention is provided. For treatment of those infections, conditions or disease states which are specific for a specific animal such as a human patient, the term patient refers to that specific animal. In most instances, the patient or subject of the present invention is a human patient of either or both sexes.

The term “effective” is used herein, unless otherwise indicated, to describe a number of VLP's or an amount of a VLP-containing composition which, in context, is used to produce or effect an intended result, whether that result relates to the prophylaxis and/or therapy of a Chlamydia trachomatis infection or morbidity/symptom thereof as otherwise described herein. The term effective subsumes all other effective amount or effective concentration terms (including the term “therapeutically effective”) which are otherwise described or used in the present application.

Accordingly, VLPs can be used as a platform to elicit rapid, high-titer, and long-lasting antibody responses to Chlamydia trachomatis (Ct) infection. These features are required for effective vaccine-based treatment for Chlamydia trachomatis (Ct) infection. In embodiments, the present vaccines provide an unexpectedly quick immunogenic response, which is strong and long lasting response to which represents an unexpected result.

In an embodiment, the present invention is directed to a composition comprising: (a) a virus-like particle (VLP) comprising a bacteriophage coat protein; and (b) at least one conjugated or inserted peptide determinant; wherein said peptide determinant is displayed on said virus-like particle, and wherein said determinant comprises a conjugated immunogenic peptide of at least five contiguous amino acids of a peptide sequence according to the peptides of SEQIDNOS:1-75 hereof or as otherwise described herein. In embodiments, the immunogenic peptide is conjugated to the surface of the VLP at high density. In an embodiment, the immunogenic peptide conjugate determinant is displayed at one or more lysine residues of said bacteriophage coat protein.

In embodiments, the bacteriophage coat protein used to form the VLPs is a coat protein derived from Qbeta, MS2, or AP205 bacteriophage as a monomeric unit, preferably a monomeric coat protein derived from Qbeta bacteriophage.

In embodiments, in the composition according to the present invention the immunogenic peptide conjugate determinant is displayed at one or more nucleophilic or electrophilic amino acid residues on the surface of the bacteriophage, preferably at a plurality of lysine residues on the surface of the VLP. In embodiments, the immunogenic peptide conjugate is displayed on the bacteriophage at the lysine residues by covalently binding an immunogenic peptide as described herein to the lysine residues through a linker group. In embodiments, the linker group comprises a 4 to 15 mer, preferably a 4 to 10 mer oligopeptide covalently bonded to a crosslinker which is bonded to the VLP particle as described herein.

In embodiments of the present invention, the oligopeptide linker of the immunogenic peptide conjugate is covalently bonded to an electrophilic or nucleophilic group of the crosslinker (e.g. a carbonyl group, a vinyl group, an amine or hydroxyl group) which optionally has been modified to facilitate the binding of the oligopeptide and immunogenic peptide to the crosslinker and the crosslinker is bonded to the nucleophilic or electrophilic amino acid residues, preferably lysine residues on the surface of the bacteriophage through the crosslinker, which may be optionally modified to promote covalent binding between the bacteriophage and the crosslinker (which is linked through the oligopeptide linker to the immunogenic peptide). In embodiments, the oligopeptide of the linker is a 4 to 15 mer, preferably a 4 to 10 mer oligopeptide comprising neutral amino acid residues bonded to nucleophilic or electrophilic sites of the immunogenic peptide (which is often an amine group or carboxylic acid group of the immunogenic peptide). In embodiments, on one end of the oligopeptide, often the carboxyl terminus, the oligopeptide comprises a cysteinyl group or other amino acid which may be used to link the oligopeptide to the crosslinker. The amino end of the oligopeptide may optionally be conjugated to the immunogenic peptide molecule through the use of a short amide linker (e.g. a C₁-C₄ alkyl amide group which forms a urea or urethane group with the peptide radical) or other group, among others. At the other end of the oligopeptide linker, often the oligopeptide linker is conjugated to the immunogenic peptide by forming a covalent bond with the amine terminus or carboxyl terminus of the immunogenic peptide.

In embodiments, the neutral amino acid residues are selected from the group consisting of glycine, alanine, valine, leucine, isoleucine, phenylalanine, tryptophan, methione, proline, serine and mixtures thereof. Embodiments, the neutral amino acids often are selected from the group consisting of glycine, serine and mixtures thereof, more often glycine.

In embodiments, the immunogenic peptide comprises at least five contiguous amino acids of the immunogenic peptide sequence of SEQIDNOS. 1-75, often SEQIDNOs: 25-29, 54-58 or 71-75.

In embodiments, the present invention is directed to a population of virus-like particles as otherwise described herein.

In embodiments, the present invention is directed to a pharmaceutical composition comprising a population of virus-like particles as described herein in combination with a pharmaceutically acceptable carrier, additive and/or excipient, or alone. In embodiments, the composition is formulated for administration to a subject or patient as a vaccine. In embodiments the pharmaceutical composition or vaccine comprises an adjuvant (e.g., Advax, MF 59, CPG 1018, AS01B, AS03, AS04, etc.).

In embodiments, the present invention is directed to a method for enhancing an immune response against a Chlamydia trachomatis (Ct) infection in a patient or subject in need comprising introducing a pharmaceutical composition comprising a population of VLPs as otherwise described herein to said subject or patient, wherein an enhanced immune response against said Chlamydia trachomatis (Ct) infection is produced in said patient or subject. In embodiments, the present invention is directed to a method for reducing the likelihood of a Chlamydia trachomatis (Ct) infection or associated morbidity in a patient or subject in need. In embodiments, the present invention is directed to a method wherein the composition is prophylactic for a Chlamydia trachomatis (Ct) infection.

In embodiments, the present invention is direct to a method of inducing an immunogenic response in a patient or subject comprising administering a composition comprising an effective amount of a population of immunogenic peptide VLPs as otherwise described herein to said patient or subject.

In embodiments, the present invention is directed to a method for treating or inhibiting a Chlamydia trachomatis (Ct) infection, Ct transmission, morbidity, or a symptom thereof in a patient or subject in need comprising administering to said patient or subject a composition comprising an effective amount of a population of immunogenic peptide conjugated VLPs as otherwise described herein to said patient or subject.

In embodiments, the infection is a Chlamydia trachomatis (Ct) infection and the symptom is a symptom associated with said infection, such as discharge and/or bleeding from the vagina, penis or rectum of a subject, painful urination, painful sexual intercourse, bleeding between periods and/or after sexual intercourse in women and testicular pain in men. Morbidity is one or more of pelvic inflammatory disease, ectopic pregnancy, fallopian tube scarring, upper genital tract inflammation, cervicitis, and tubal factor infertility.

In embodiments, the present invention is directed to a method for treating or reducing the likelihood of a Chlamydia trachomatis (Ct) infection, morbidity, or a symptom thereof in a patient or subject in need comprising administering to said patient a composition comprising an effective amount of a population of immunogenic peptide conjugated VLPs as otherwise described herein to said patient or subject. In embodiments, the symptom is one or more of discharge and/or bleeding from the vagina, penis or rectum of a subject, painful urination, painful sexual intercourse, bleeding between periods and/or after sexual intercourse in women and testicular pain in men. In embodiments, the morbidity is one or more of pelvic inflammatory disease, ectopic pregnancy, fallopian tube scarring, and tubal factor infertility.

The present invention is therefore directed to vaccines which target Chlamydia trachomatis (Ct) infection or a symptom/morbidity thereof in a patient or subject in need as otherwise disclosed herein for prophylactic and/or therapeutic purposes.

As used herein, the term “immunogenic peptide” is used to describe a peptide which comprises a peptide determinant (i.e. an antigenic determinant or epitope) which interacts with and raises antibodies for an immunogenic response. These peptides are chemically conjugated with VLPs or genetically inserted into VLP coat proteins according to the present invention in order to produce an immunogenic response in a subject at risk for or challenged by a Chlamydia trachomatis (Ct) infection. Immunogenic peptides for use in the present invention are include any immunogenic peptide of at least 5 contiguous amino acids (between 5 and 25 contiguous amino acids of the peptide sequences which are set forth as SEQIDNOS:1-75, often SEQIDNOS: 25-29, 54-58 or 71-75.

The term “immunogenic peptide conjugate” or peptide conjugate refers to an immunogenic peptide of between 5 and 25 amino acid residues in length which comprises a peptide or antigenic determinant (epitope) and is conjugated to the external surface of a VLP, often a Qβ or AP205 bacteriophage, often a Qβ bacteriophage through a linker molecule to a nucleophilic amino acid on the surface of the bacteriophage. In embodiments, the nucleophilic amino acid is a lysine residue on the surface of the bacteriophage. The immunogenic peptide is conjugated to the bacteriophage through a linker molecule. Often the linker molecule comprises a 4-15 mer, often a 4-12 mer, a 4-10 mer, a 4-8 mer a 4-6 mer or a 4 mer oligopeptide (preferably comprising neutral amide acid residues) which is covalently bonded to a crosslinker molecule at one end and the immunogenic peptide on the other end as described herein to form the linker. Accordingly, the oligonucleotide is covalently linked at one end to the immunogenic peptide often through an electrophilic or nucleophilic functional group on the immunogenic peptide (often a carboxyl group or amine group, more often an amine group which is optionally further linked by an amide or other group, often a short, C₁-C₄ alkyl amide) and on the other end to the crosslinker, which further links the VLP to the oligopeptide and the immunogenic peptide. This is shown in the examples which are described herein.

The term “crosslinker” or “crosslinking agent” refers to a chemical compound used to covalently bind, or conjugate, biomolecules together, such as an oligopeptide to a VLP, directly to a VLP or to an oligopeptide which is covalently bonded to an immunogenic peptide. The term “protein crosslinking” refers to utilizing protein crosslinkers to conjugate peptides or proteins together. Crosslinking agents for use herein possess reactive moieties specific to various electrophilic or nucleophilic functional groups (e.g., sulfhydryls, amines, carbohydrates, carboxyl groups, hydroxyl groups, carbonyls, etc.) on proteins, peptides, or other molecular complexes or molecules as described herein. The atoms separating a crosslinker agent's reactive groups, and eventually the conjugated oligopeptide/VLP or oligopeptide/immunogenic peptide form the “spacer arm”. A zero-length crosslinker refers to protein crosslinkers that join two molecules without adding additional spacer arm atoms, which is relevant and used in embodiments according to the present invention. Hotnobifunctional crosslinker reagents have the same reactive group on both ends of the spacer arm (i.e., Amine Reactive-Amine Reactive) while heterobifnctional crosslinkers have different reactive groups on each end of a spacer arm (i.e., Sulfhydryl Reactive-Amine Reactive). It is noted that in addition to the following crosslinking agents, additional short-chain crosslinking agents such as short-chain alkyl amides (CH₂)ᵢC(O)NH₂, (CH₂)ᵢC(O), C(O)(CH₂)ᵢC(O), NHC(O)(CH₂)ᵢC(O) or NHC(O)(CH₂)ᵢC(O)NH groups where i is from 1 to 4, can be used to link an immunogenic peptide to an oligopeptide or a crosslinker to a lysine group on the VLP. The following crosslinking agents are exemplary for use in the present invention:
ANB-NOS (N-5-Azido-2-nitrobenzoyloxysuccinimide)
BMPS N-(β-Maleimidopropyloxy)succinimide ester
EMCS (N[e-Maleimidocaproyloxy]succinimide ester)
GMBS (N[Gamma-Maleimidobutyryloxy] Succinimide)
LC-SPDP Succinimidyl 6-(3[2-pyridyldithio]-propionamido)hexanoate)
MBS (in-Maleinaidobenzoyl-N-hydroxysuccinimide ester)
PDPH (3[2-Pyridyldithio]propionyl hydrazide)
SBA (N-Succinimidyl bromoacetate)
SIA (N-Succinimidyl iodoacetate)
Sulfo-SIA N-Sulfosuccinimidyl iodoacetate)
SMCC (Succinimidyl-4[N-maleirnidomethyl]cyclohexane-1-carboxylate)
SMPB (N-Succinimidyl 4[4-maleimidophenyl]butyrate)
SMPH (Succininaidyl-6[β-maleimidopropionamido]hexanoate)
SPDP (N-Succinimidyl 3[2-pyridyldithio]-propionate)
Sulfo-LC-SPDP Sulfosuccinimidyl 6-(3′[2-pyridyldithio]-propionamido)hexoate
Sulfo-MBS (m-Maleimidobenzoyl-N-hydroxysulfosuccinimide ester)
Sulfo-SANPAH (N-Sulfosuccinimidyl-6[4′-azido-2′-nitrophenylamino]hexanoate)
sulfo-SMCC (Sulfosuccinimidyl-4-[N-maleimidomethyl]cyclohexane-l-carboxylate)
BS2G (Bis[Sulfosuccinimidyl] glutarate)
BS3 (Bis[sulfosuccinimidyl] suberate)
DSG (Disuccinimidyl glutarate)
DSP (Dithiobis[succininudyl propionate])
DSS (Disuccinimidyl suberate)
DSSeb (Disuccinirnidyl sebacate)
DST (Disuccinimidyl tartrate)
DTSSP (3,3′-Dithiobis[sulfosuccinimidylpropionate])
EGS (Ethylene glycolbis(succinimidylsuccinate)
Sulfo-EGS Ethylene glycolbis(sulfosuccinimidylsuccinate)
CDI (N,N′-Carbonyldiimidazole)
DCC (N,N′-dicyclohexylcarbodihnide)
EDC-HCl 1-Ethyl-3-[3-dimethylaminopropyl]carbodiimide hydrochloride)
NHS (N-hydroxysuccinimide) and
Sulfo-NHS (N-hydroxysulf7osuccinimide).

Crosslinkers which also may be used in the present invention are heterobifunctional agents which are capable of linking Amine-to-Sulfhydryl groups. Exemplary crosslinking agents include:
SIA (succinimidyl iodoacetate)
SBAP (succinimidyl 3-(bromoacetamido)propionate)
SIAB (succinimidyl (4-iodoacetyl)aminobenzoate)
Sulfo-SIAB (sulfosuccinimidyl (4-iodoacetyl)aminobenzoate)
AMAS (N-α-maleimidoacet-oxysuccinimide ester)
BMPS (N-β-maleimidopropyl-oxysuccinimide ester)
GMBS (N-y-maleimidobutyryl-oxysuccinimide ester)
MBS (m-maleimidobenzoyl-N-hydroxysuccinimide ester)
SMCC (succinimidyl 4-(N-maleimidomethyl)cyclohexane-1-carboxylate)
EMCS (N-c-malemidocaproyl-oxysuccinimide ester)
Sulfo-GMBS (N-y-maleimidobutyryl-oxysulfosuccinimide ester)
Sulfo-MBS (m-maleimidobenzoyl-N-hydroxysulfosuccinimide ester)
Sulfo-SMCC (sulfosuccinimidyl 4-(N-maleimidomethyl)cyclohexane-1-carboxylate)
Sulfo-EMCS (N-c-maleimidocaproyl-oxysulfosuccinimide ester)
Sulfo-SMPB (sulfosuccinimidyl 4-(N-maleimidophenyl)butyrate)
SMPB (succinimidyl 4-(p-maleimidophenyl)butyrate)
SMPH (Succinimidyl 6-((beta-maleimidopropionamido)hexanoate))
LC-SMCC (succinimidyl 4-(N-maleimidomethyl)cyclohexane-l-carboxy-(6-amidocaproate)) and
Sulfo-KMUS (N-K-maleimidoundecanoyl-oxysulfosuccinimide ester).

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, such as coding regions, and non-coding regions such as regulatory sequences (e.g., promoters or transcriptional terminators). A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

The term “single-chain dimer” refers to a normally dimeric protein whose two subunits of coat polypeptide of a RNA bacteriophage have been genetically (chemically, through covalent bonds) fused into a single polypeptide chain. Specifically, in the present invention single-chain versions of bacteriophage are constructed. Each of these proteins is naturally a dimer of identical polypeptide chains. In certain of the bacteriophages coat protein dimers of the N-terminus of one subunit lies in close physical proximity to the C-terminus of the companion subunit. Single-chain coat protein dimers may be produced using recombinant DNA methods by duplicating the DNA coding sequence of the coat proteins and then fusing them to one another in tail to head fashion. The result is a single polypeptide chain in which the coat protein amino acid appears twice, with the C-terminus of the upstream copy covalently fused to the N-terminus of the downstream copy. Normally (wild-type) the two subunits are associated only through noncovalent interactions between the two chains. In the single-chain dimer these noncovalent interactions are maintained, but the two subunits have additionally been covalently tethered to one another. This greatly stabilizes the folded structure of the protein and confers to it its high tolerance of peptide insertions as described above.

In preferred embodiments, the coat polypeptide of the VLP, often monomeric Qbeta, MS2, or AP205 coat polypeptide, most often Qbeta, is prepared and the bacteriophage coat polypeptides self-assemble into VLPs typically comprising 180 copies of the coat polypeptide for each VLP.

The RNA phage coat proteins possess a conserved tertiary structure. Immunogenic peptides conjugated at the N- or C-terminus are exposed on the surface of the VLP and are strongly immunogenic.

The amino acid residues described herein are preferred to be in the “L” isomeric form. However, residues in the “D” isomeric form can be substituted for any L-amino acid residue, as long as the desired functional is retained by the polypeptide. NH 2 refers to the free amino group present at the amino terminus of a polypeptide. COOH refers to the free carboxy group present at the carboxy terminus of a polypeptide.

The term “valency” is used to describe the density of the immunogenic peptide conjugates displayed on VLPs according to the present invention. Valency in the present invention may range from low valency to high valency (“high density”), i.e., from less than 1 to more than about 180, preferably 90 to 180 or in certain cases more (e.g between 90-720, or between 1 and 4 conjugates per coat protein in the VLP). Immunogenic compositions according to the present invention comprise VLPs which are preferably high valency and comprise VLPs which display at least 50-60 up to about 180 or more, often 50-180 or more, more often 90-180 or more crosslinked conjugated immunogenic peptides per VLP as otherwise described herein. In embodiments, at least 90 immunogenic peptide conjugates are “high density” because of the display of 90 copies of antigen/immunogenic peptide on the surface of the VLP produces high titer antibodies.

The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) located just upstream of the open reading frame at the 5′-end of the mRNA and a transcription terminator sequence located just downstream of the open reading frame at the 3′-end of the mRNA. A coding sequence can include, but is not limited to, DNA, cDNA, and recombinant nucleic acid sequences.

A “heterologous” region of a recombinant cell is an identifiable segment of nucleic acid within a larger nucleic acid molecule that is not found in association with the larger molecule in nature.

An “origin of replication” refers to those DNA sequences that participate in DNA synthesis.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the -10 and -35 consensus sequences.

In bacteria, transcription normally terminates at specific transcription termination sequences, which typically are categorized as rho-dependent and rho-independent (or intrinsic) terminators, depending on whether they require the action of the bacterial rho-factor for their activity. These terminators specify the sites at which RNA polymerase is caused to stop its transcription activity, and thus they largely define the 3′-ends of the RNAs, although sometimes subsequent action of ribonucleases further trims the RNA.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

An “antibiotic resistance gene” refers to a gene that encodes a protein that renders a bacterium resistant to a given antibiotic. For example, the kanamycin resistance gene directs the synthesis of a phosphotransferase that modifies and inactivates the drug. The presence on plasmids of a kanamycin resistance gene provides a mechanism to select for the presence of the plasmid within transformed bacteria. Similarly, the chloramphenicol resistance gene allows bacteria to grow in the presence of the drug by producing an acetyltransferase enzyme that inactivates the antibiotic through acetylation.

The term “PCR” refers to the polymerase chain reaction, a technique used for the amplification of specific DNA sequences in vitro. The term “PCR primer” refers to DNA sequences (usually synthetic oligonucleotides) able to anneal to a target DNA, thus allowing a DNA polymerase (e.g. Taq DNA polymerase) to initiate DNA synthesis. Pairs of PCR primers are used in the polymerase chain reaction to initiate DNA synthesis on each of the two strands of a DNA and to thus amplify the DNA segment between two primers. Representative PCR primers which are used in the present invention are those which are presented in the examples section hereof.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid, which normally replicate independently of the bacterial chromosome by virtue of the presence on the plasmid of a replication origin. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

It should be appreciated that also within the scope of the present invention are nucleic acid sequences encoding the polypeptide(s) of the present invention, which code for a polypeptide having the same amino acid sequence as the sequences disclosed herein, but which are degenerate to the nucleic acids disclosed herein. By “degenerate to” is meant that a different three-letter codon is used to specify a particular amino acid.

As used herein, “epitope” refers to an antigenic determinant of a polypeptide. An epitope could comprise 3 amino acids in a spatial conformation which is unique to the epitope. Generally an epitope consists of at least 5 such amino acids, and more often, consists of least 5-25, often 5-20, more often 5-15 or 5-10 such amino acids. Methods of determining the spatial conformation of amino acids are known in the art, and include, for example, x-ray crystallography and 2-dimensional nuclear magnetic resonance.

As used herein, the term “coat protein(s)” refers to the protein(s) of a bacteriophage or a RNA-phage capable of being incorporated within the capsid assembly of the bacteriophage or the RNA-phage. These include, but are not limited to Qβ, PP7, MS2, AP205, R17, SP, PP7, GA, M11, MX1, f4, CbS, Cb12r, Cb23r, 7s and f2 RNA bacteriophages. Preferred coat proteins which are used in the present invention include coat proteins from bacteriophages include Qβ, AP205, PP7 and MS2. Preferably, Qβ or AP205, most often Qβ coat polypeptides, often Qβ coat polypeptide dimers are used to create conjugated VLPs according to the present invention.

As used herein, a “coat polypeptide” as defined herein is a polypeptide of the full length coat protein of the bacteriophage, a polypeptide fragment of the coat protein that possesses coat protein function and additionally encompasses the full length coat protein as well or single-chain variants thereof

As used herein, the term “immune response” refers to a humoral immune response and/or cellular immune response leading to the activation or proliferation of B- and/or T-lymphocytes and/or antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic peptide” is a conjugated peptide that elicits a cellular and/or humoral immune response as described above, whether alone or linked to a carrier in the presence or absence of an adjuvant. Preferably, antigen presenting cell may be activated.

As used herein, the term “vaccine” refers to a formulation which contains the composition of the present invention and which is in a form that is capable of being administered to an animal, often a human patient or subject.

As used herein, the term “virus-like particle of a bacteriophage” refers to a virus-like particle (VLP) resembling the structure of a bacteriophage, being non-replicative and noninfectious, and lacking at least the gene or genes encoding for the replication machinery of the bacteriophage, and typically also lacking the gene or genes encoding the protein or proteins responsible for viral attachment to or entry into the host.

This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA bacteriophage coat protein: The capsid structure formed from the self-assembly of one or more subunits of RNA bacteriophage coat protein and optionally containing host RNA is referred to as a “VLP of RNA bacteriophage coat protein”. In a particular embodiment, the capsid structure is formed from the self assembly of 90 coat protein single-chain dimers or 180 coat protein monomers. In the case of Qβ or AP205 VLPs 90 coat protein dimers or 180 coat protein monomers typically self-assemble into the VLP.

A nucleic acid molecule is “operatively linked” to, or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

### Immunogenicity and Prophylactic Efficacy

Chlamydia trachomatis (Ct) infection and related symptoms and morbidity includes, but is not limited to, the symptoms identified in this application which are caused by Chlamydia trachomatis (Ct) infection. Immunogenicity and prophylactic efficacy (e.g. whether a composition is prophylactic for Chlamydia trachomatis (Ct) infection) may be evaluated either by the techniques and standards mentioned in the attached APPENDIX A, or through other methodologies that are well-known to those of ordinary skill in the art.

To assess immunogenicity (e.g. whether a composition has induced a high-titer antibody responses against Chlamydia trachomatis (Ct) or the peptide conjugated to the surface of the VLP, an anti-Chlamydia trachomatis (Ct) geometric mean titer (GMT) can be measured by ELISA, e.g. after a few weeks of treatment (e.g. 3 or 4 weeks) and after administration of a few dosages (e.g. 2 or 3). The percentage of subjects who seroconverted for Chlamydia trachomatis (Ct) antigenicity (OA) after a few weeks of treatment (e.g. 3 or 4 weeks) and after administration of a few dosages (e.g. 1 to 4) can also be determined to assess immunogenicity.

### Production of Virus-Like Particles

The present invention is directed to virus-like phage particles as well as methods for producing these particles in vivo as well as in vitro. As used herein, producing virions “in vitro” refers to producing virions outside of a cell, for instance, in a cell-free system, while producing virions “in vivo” refers to producing virions inside a cell, for instance, an Escherichia coli or Pseudomonas aeruginosa cell or a yeast cell among others.

### Bacteriophages

The VLPs described here consist of assemblies of the coat proteins of single-strand RNA bacteriophage [RNA Bacteriophages, in The Bacteriophages. Calendar, RL, ed. Oxford University Press. 2005]. The known viruses of this group infect bacteria as diverse as E. coli, Pseudomonas and Acinetobacter. Each possesses a highly similar genome organization, replication strategy, and virion structure. In particular, the bacteriophages contain a single-stranded (+)-sense RNA genome, contain maturase, coat and replicase genes, and have small (<300 angstrom) icosahedral capsids. These include but are not limited to Qβ, AP205, PP7, MS2, R17, SP, PP7, GA, M11, MX1, f4, CbS, Cb12r, Cb23r, 7s and f2 RNA bacteriophages. Qβ, MS2, and AP205 RNA bacteriophages are preferred, Qβ is most preferred. QP and AP205 RNA bacteriophages form self-assembled VLPs from 90 dimeric coat polypeptide units or 180 monomeric coat polypeptide units. Methods for producing these coat polypeptides are well known in the art. See, for example Freivalds, et al., J Biotechnol., 2006 May 29;123(3):297-303.

The information required for assembly of the icosahedral capsid shell of this family of bacteriophage is contained entirely within coat protein itself. For example, purified coat protein can form capsids in vitro in a process stimulated by the presence of RNA [Beckett et al., 1988, J. Mol Biol 204: 939-47]. Moreover, coat protein expressed in cells from a plasmid assembles into a virus-like particle in vivo [Peabody, D.S., 1990, J Biol Chem 265: 5684-5689].

The preferred VLP for use in the present invention is a Qbeta or QP VLP. These VLPs are typically made by transformation of E. coli with a plasmid expressing the Qbeta coat protein as a monomer under a lac promoter. Colonies are selected on kanamycin Luria Broth (LB) agar plates. A single colony is used to inoculate LB broth and grown overnight at 37 degrees C. This is then used to inoculate a larger culture. Cultures are shaken at 37 degrees C. for several hours until OD600 reaches 0.8. Then the expression of the Qbeta coat protein is induced with isopropyl β-d-1-thiogalactopyranoside (IPTG) and incubated another 3 hours. Then cells are pelleted and frozen at −20 degrees C. Lysis of bacteria is then performed in isotonic buffer with sonication. VLPs are isolated by size exclusion chromatography and endotoxin is depleted with sequential Triton-X-100 phase extraction. There are many alternative methods to isolate the VLPs, which are well known in the art. The examples section of the present application describe the formation and production of Qβ VLPs.

### RNA Bacteriophage Coat Polypeptide

The coat polypeptides useful in the present invention also include those having similarity with one or more of the coat polypeptide described above. The similarity is referred to as structural similarity. Structural similarity may be determined by aligning the residues of the two amino acid sequences (i.e., a candidate amino acid sequence and the amino acid sequence) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence can be isolated from a single stranded RNA virus, or can be produced using recombinant techniques, or chemically or enzymatically synthesized. Preferably, two amino acid sequences are compared using the BESTFIT algorithm in the GCG package (version 10.2, Madison WI), or the Blastp program of the BLAST 2 search algorithm, as described by Tatusova, et al. (FEMS Microbial Lett 1999, 174:247-250), and available at http://www.ncbi.nlm.nih.gov/blast/b12seq/b12.html. Preferably, the default values for all BLAST 2 search parameters are used, including matrix=BLOSUM62; open gap penalty=11, extension gap penalty=1, gap xdropoff=50, expect=10, wordsize=3, and optionally, filter on. In the comparison of two amino acid sequences using the BLAST search algorithm, structural similarity is referred to as “identities.” Preferably, a coat polypeptide also includes polypeptides with an amino acid sequence having at least 80% amino acid identity, at least 85% amino acid identity, at least 90% amino acid identity, or at least 95% amino acid identity to one or more of the amino acid sequences disclosed above. Often, a coat polypeptide is inactive. Whether a coat polypeptide is active can be determined by evaluating the ability of the polypeptide to form a capsid and package a single stranded RNA molecule. Such an evaluation can be done using an in vivo or in vitro system, and such methods are known in the art and routine. Alternatively, a polypeptide may be considered to be structurally similar if it has similar three-dimensional structure as the recited coat polypeptide and/or functional activity.

### The Immunogenic Peptide Conjugate

As described herein, in certain embodiments the immunogenic conjugate may be present (covalently linked) to the VLP in the A-B loop, at the N-terminus or the carboxy terminus of a coat polypeptide. Preferably, the oligopeptide conjugate is covalently linked on the outer surface of the capsid. In particularly preferred embodiments, the approach is to attach the conjugated peptides to lysine residues present on the surface of Qbeta

### Qbeta VLPs Lysine Positions

The lysine residues which are available for conjugation on the coat polypeptide of the Qbeta VLPs of the present invention are set forth

```
    1 akletvtlgn igkdgkqtlv Inprgvnptn
        gvaslsqaga vpalekrvtv svsqpsrnrk
  61 nykvqvkiqn ptactangsc dpsvtrqaya
        dvtfsftqys tdeerafvrt elaallaspl
121 lidaidqlnp ay
```

Lysine amino acid residues are indicated above in bold. They are at amino acid positions 2, 13, 16, 46, 60, 63, and 67 of the monomeric coat polypeptide.

In embodiments, the present invention is directed to A-B loop, N-terminal or C-terminal presentation of peptide conjugates on VLPs including PP7, MS2, AP205 and Qβ, preferably Qβ. These VLP-CPs (VLP-conjugated peptides) can be used singly or as a combination vaccine. The inventors show protection against Chlamydia trachomatis (Ct) infection in cell culture using a vaccine consisting of a Qbeta VLP conjugated peptide wherein the immunopeptide is conjugated to lysine residues on the Qbeta VLP as described herein.

In a particular embodiment, the coat polypeptide is a single-chain dimer containing an upstream and downstream subunit. Each subunit contains a functional coat polypeptide sequence. The peptide conjugate may be inserted in the upstream and/or downstream subunit at the sites mentioned herein above, e.g., the A-B loop, the N-terminus or a carboxyl terminus. In a particular embodiment, the coat polypeptide is a single chain dimer of a Qβ, PP7 or MS2 coat polypeptide, preferably a QP coat polypeptide, although a number of bacteriophage coat polypeptides may be used.

### Preparation of Transcription Unit

The transcription unit of the present invention comprises an expression regulatory region, (e.g., a promoter), a sequence encoding a coat polypeptide and transcription terminator. The RNA polynucleotide may optionally include a coat recognition site (also referred to a “packaging signal”, “translational operator sequence”, “coat recognition site”). Alternatively, the transcription unit may be free of the translational operator sequence. The promoter, coding region, transcription terminator, and, when present, the coat recognition site, are generally operably linked. “Operably linked” or “operably associated with” refer to a juxtaposition wherein the components so described are in a relationship permitting them to function in their intended manner. A regulatory sequence is “operably linked” to, or “operably associated with”, a coding region when it is joined in such a way that expression of the coding region is achieved under conditions compatible with the regulatory sequence. The coat recognition site, when present, may be at any location within the RNA polynucleotide provided it functions in the intended manner.

The invention is not limited by the use of any particular promoter, and a wide variety of promoters are known. The promoter used in the invention can be a constitutive or an inducible promoter. Preferred promoters are able to drive high levels of RNA encoded by the coding region encoding the coat polypeptide. Examples of such promoters are known in the art and include, for instance, the lac promoter, T7, T3, and SP6 promoters.

The nucleotide sequences of the coding regions encoding coat polypeptides described herein are readily determined. These classes of nucleotide sequences are large but finite, and the nucleotide sequence of each member of the class can be readily determined by one skilled in the art by reference to the standard genetic code. Furthermore, the coding sequence of an RNA bacteriophage single chain coat polypeptide comprises a site for covalent binding of an immunogenic peptide conjugate. In a particular embodiment, the site for insertion of the peptide conjugate is at an appropriate amino acid residue exposed on the surface of the VLP (i.e., an amino acid which contains a functional group capable of conjugation to the immunogenic conjugate).

In a particular embodiment, the coding region encodes a single-chain dimer of the coat polypeptide. In an the coding region encodes a modified single chain coat polypeptide dimer, where the modification comprises an insertion of a coding sequence at one or more amino acids at the conjugation site. The transcription unit may contain a bacterial promoter, such as a lac promoter or it may contain a bacteriophage promoter, such as a T7 promoter.

### Synthesis

The VLPs of the present invention may be produced in vivo by introducing transcription units into bacteria, especially if transcription units contain a bacterial promoter. Alternatively, it may be synthesized in vitro in a coupled cell-free transcription/translation system.

The preferred VLP for use in the present invention is a Qbeta or Qβ VLP. These VLPs are typically made by transformation of E. coli with a plasmid expressing the Qbeta coat protein as a monomer under a lac promoter. Colonies are selected on kanamycin Luria Broth (LB) agar plates. A single colony is used to inoculate LB broth and grown overnight at 37 degrees C. This is then used to inoculate a larger culture. Cultures are shaken at 37 degrees C. for several hours until OD600 reaches 0.8. Then the expression of the Qbeta coat protein is induced with Isopropyl β-d-1 -thiogalactopyranoside (IPTG) and incubated another 3 hours. Then cells are pelleted and frozen at −20 degrees C. Lysis of bacteria is then performed in isotonic buffer with sonication. VLPs are isolated by size exclusion chromatography and endotoxin is depleted with sequential Triton-X-100 phase extraction. There are many alternative methods to isolate the VLPs, which are well known in the art.

### Assembly of VLPs Encapsidating Heterologous Substances

As noted above, the VLPs of the present invention conjugate immunogenic peptides on the surface of the VLPs. These VLPs may be assembled and combined with another substance, such as an adjuvant, or another VLP, such as heterologous prime boost strategies. Specifically, purified coat protein subunits are obtained from VLPs that have been disaggregated with a denaturant (usually acetic acid). The adjuvant is mixed with coat protein, which is then reassembled in its presence. In a particular embodiment, the substance has some affinity for the interior of the VLP and is preferably negatively charged.

In another embodiment, the adjuvant is passively diffused into the VLP through pores that naturally exist in the VLP surface. In a particular embodiment, the substance is small enough to pass through these pores and has a high affinity for the interior of the VLP.

In embodiments, the present invention is directed to a population of virus-like particles (VLPs) to which are conjugated immunopeptides as described herein. In embodiments, the population of conjugated VLPs is combined with a pharmaceutical carrier, additive and/or excipient to provide immunogenic compositions according to the present invention. In embodiments the population of VLPs is formulated into a pharmaceutical composition as a vaccine formulation (vaccine) for immunizing a patient or subject against a Chlamydia trachomatis (Ct) infection. In embodiments, the pharmaceutical composition comprises an adjuvant or other active component to enhance or facilitate an immunogenic response in a patient or subject to which the composition is administered. The compositions often include a pharmaceutically acceptable carrier, additive or excipient. These include preservatives, to prevent contamination, salts, buffering agents, surfactants, amino acids, stabilizers, such as various sugars or gelatin to keep the vaccine potent during transportation and storage, cell culture materials, used to grow the vaccine antigens, such as egg protein, culture media, inactivating ingredients, for example, formaldehyde used to kill viruses or inactivate toxins and antibiotics to prevent contamination and bacterial grown, among others. These are well-known in the art.

The invention is described in additional detail in the accompanying description and examples presented herein below.

### Overall Aims of and Approaches to the Present Invention

Chlamydia trachomatis (Ct) is the most common bacterial sexually transmitted infection. Long-term sequelae of Ct infection in women include pelvic inflammatory disease, ectopic pregnancy, and tubal factor infertility. Ct infections continue to increase world-wide despite widespread screening programs and the availability of curative antibiotic treatment. The WHO and the NIH have both prioritized vaccine development against Ct. Despite over 70 years of effort, there is no licensed vaccine available against Chlamydia. A viable, effective vaccine would therefore meet a long-felt need in the art. Some of the challenges that have hindered vaccine development include: (1) identifying appropriate vaccine targets, (2) choosing the appropriate model in which to assess a vaccine, (3) eliciting appropriate protective immune responses, and (4) the immunopathogenic nature of the natural immune response to Ct in women. Female urogenital infection with Ct is characterized by immune-mediated damage to the upper reproductive tract. Because of the immunopathogenic nature of Ct infection in women, the vaccine strategy used to develop the present invention focused on eliciting protective yet non-pathogenic responses. Since the natural protective immune response in humans appears to be mediated by IFN-γ secreting CD4+ T cells, most vaccine strategies have attempted to reproduce this type of response with a vaccine. Ct vaccine approaches focused on eliciting antibody responses might provide an alternative and innovative approach to the field's longstanding focus on eliciting cell-mediated immunity. One benefit of this approach is that it would eliminate any safety concerns due to the elicitation of inappropriate T cell responses (like CD8+ T cell responses secreting TNF-alpha).

The overall goal of the research that led to the present invention is to design prophylactic vaccines against urogenital Ct infection by identifying immunogens that can elicit epitope-specific antibodies able to protect against urogenital Ct infection and pathogenesis. Using a highly immunogenic bacteriophage virus-like particle (VLP) vaccine platform, the inventors identified epitope-specific antibody responses that can protect against Ct infection and identified peptide epitopes of interest, which are the targets of antibodies that bind to Ct elementary bodies (EBs), neutralize Ct infection of human cells in vitro or have other functionality, and are immunogenic in women with a history of urogenital Ct infection. Our data show the feasibility of our approach in reducing Chlamydia urogenital infection in female mice. In this work the inventors help to identify prophylactic Chlamydia vaccine candidates to move into further testing, and also to help define the range of functions antibodies can have against Chlamydia infection of the female urogenital tract.

### Engineering Epitope-Specific Immunogens to Chlamydia

The inventors engineered vaccines by using a bacteriophage virus-like particle (VLP) platform to display Ct peptides of interest. These VLPs are highly immunogenic, eliciting long-lasting, high-titer antibodies to peptide epitopes displayed on their surface. The present invention focuses on antigens involved in Ct adhesion and entry into host cells (such as PmpC, PmpD, MOMP, OmcB). Additionally, the inventors advanced previous data and identified new peptide epitopes that are immunogenic in women infected with urogenital Ct. The inventors focused on several bacteriophage VLP platforms, allowing the inventors to target both short peptide epitopes and larger antigen epitopes, but directed their principal focus on VLPs comprising Qβ coat polypeptide dimers, which self-assemble into VLPs to which immunogenic peptides can be readily conjugated.

### Identifying the Functionality of Antibodies to Chlamydia In Vitro

The inventors defined the mechanisms by which various epitope-specific antibodies act against Chlamydia. Using the immunogens generated as described above, rabbits are immunized and the resulting immune sera tested for neutralization, complement-mediated killing, and phagocyte uptake and killing activities against Ct using in vitro assays. This approach filled an important gap in the knowledge of the functions of antibodies during natural infection, and informed vaccine design by defining the correlates of antibody-mediated protection against urogenital Chlamydia.

### Assess the Protective Capacity of Antibodies Against Chlamydia In Vivo

Further experiments investigated the protective potential of specific antibody responses to Chlamydia using mouse models of infection. Mice immunized with VLPs displaying epitopes of interest were assessed for the ability of Chlamydia to infect and cause pathology in the reproductive tract of female mice. Also assessed, was the ability of the conjugated VLPs to protect female mice against sexual transmission of Chlamydia from infected male mice. This helped define epitope-specific antibody responses that can protect against urogenital Chlamydia infection, and provided VLP-based vaccine candidates to move into further testing.

### Example (MOMP Antigens) First Set of References

The inventors hypothesized that high-titer, vaccine-induced antibody responses toward a key Ct adhesion factor would provide protection against urogenital infection by blocking Ct adhesion and subsequent infection of host cells. The Major Outer Membrane Protein (MOMP) is an important adhesion factor of Ct, responsible for attachment and entry into host cells⁴⁰⁻⁴³. Indeed, MOMP is the most highly surface-expressed antigen, and it is an immunodominant antigen in animal models as well as humans⁴⁴⁻⁴⁸. In particular, the conserved region of the variable domain 4 (VD4) epitope (TTLNPTIAG) is the most immunodominant epitope, both of MOMP and overall⁴⁴. The inventors, and others, have identified this epitope as a target of interest^{32,33,49-52}. The conserved amino acid nature of this epitope across urogenital Ct serovars within an otherwise highly variable region, points toward functional importance for Ct. Further, the conserved nature, when targeted properly by a vaccine, could lend well to providing cross-protection against all urogenital serovars.

The inventors utilized the bacteriophage Qβ virus-like particle (VLP) as a vaccine platform, leveraging surface-exposed lysines to chemically conjugate the conserved VD4 epitope to the surface in a dense array⁵³⁻⁵⁵. The Qβ VLP vaccine platform induces high-titer antibodies to both the conjugated peptide and the vaccine platform. However, when short peptides are displayed on the Qβ VLP, T cell responses are typically limited to the vaccine platform alone, mediating antibody class-switching, without inducing peptide epitope-specific T cell responses⁵⁶. Here, the inventors demonstrate that immunization induces high-titer, long-lasting peripheral IgG serum antibodies, which are also present at the vaginal mucosa. Vaccine-induced antibodies are able to bind to all human Ct serovars via in vitro elementary body ELISA. Further, Qβ-VD4 vaccination can prevent vaginal Chlamydia infection, with protection that is afforded by antibody responses.

### MATERIALS AND DATA AVAILABILITY

### Production of Qβ VLPs

Escherichia coli C41 electrocompetent cells (Sigma) underwent transformation to include a plasmid containing the Qβ coat protein, under Kanamycin antibiotic selection. E. coli expressing said plasmid was then grown in lysogeny broth (LB) with shaking at 37° C. Culture was induced using 0.5 mM Isopropyl β-D-1-thiogalactopyranoside (IPTG) once the optical density (OD₆₀₀) reached 0.6-0.8. Induction continued for 3 hours at 37° C. with shaking before cells were pelleted via centrifugation and frozen at −80° C. Thawed cells were lysed via exposure to lysozyme solution (1 hour at 4° C.), sonication at 30% power multiple times, and incubation with 10 mg/mL DNase and 2mM MgCl₂ (1 hour at 37° C.). Cell debris was pelleted via centrifugation and supernatants collected. Ammonium sulfate is added to supernatants at 60% saturation and incubated overnight at 4° C. Centrifugation (10,000 RPM) then occurs, with supernatants discarded and pellets resuspended. Centrifugation is repeated, with supernatants containing Qβ VLP collected, twice more. Supernatants are frozen at −80° C. until isolation occurs. Qβ VLPs are then isolated using size exclusion chromatography via a Sepharose column. Small volume fractions are collected and ran on a 1% agarose gel to confirm Qβ VLP containing fractions. Said fractions are then run on an SDS-PAGE gel for further confirmation, along with a positive control. Fractions containing Qβ VLPs are combined and purified and concentrated using 100 K Amicon Ultra Centrifugal Filters (Merk Millipore). Purified Qβ VLPs are run again on an SDS-PAGE Gel to determine concentration using varying concentrations of Hens Egg Lysozyme controls.

### Production of Qβ VLPs Displaying MOMP VD4 Epitopes

Qβ VLP vaccines are produced by chemical conjugation of modified MOMP VD4 epitope peptides to surface exposed lysines on the coat protein of Qβ. In brief, purified Qβ VLPs are incubated for 2 hours at 25° C. with the bifunctional crosslinker Succinimidyl 6-(beta-maleimidopropionamido)-hexanoate in excess (SMPH, ThermoScientific). Excess SMPH is removed via 100 K Amicon Ultra Centrifugal filtration. Qβ-SMPH is then incubated with modified MOMP VD4 epitope peptides, containing a triglycine linker sequence and terminal cysteine (GenScript). Incubation continues at 4° C. overnight. Excess peptide is removed via 100 K Amico Ultra Centrifugal filtration. Conjugation of peptide to Qβ coat protein is confirmed via SDS-PAGE gel. Peptides representing all urogenital Ct serovars (DE, F, G, HIaJ, K) and Cm are conjugated to Qβ individually. Equal parts of the five conjugated Qβ VLPs are mixed together to produce a single mixed Ct vaccine. For NZW rabbits, 10 μg of each is added for 50 μg total/rabbit. For mice, 5 μg of each is added for 25 μg total/mouse. The Cm VD4 MOMP epitope vaccine contains only one epitope and has a final concentration of 25 μg/rabbit and 5 μl/mouse. Conjugated Qβ VLPs are assumed to have undergone half loss due to filtration and Qβ VLP vaccines are made as described in 1×PBS. Vaccines are stored at −80° C. until use.

### New Zealand White Rabbit Immunization Schedule

Female New Zealand White rabbits were purchased from Charles River Laboratories and allowed to acclimate for 3 weeks before experimentation began. At age 9-12 weeks, rabbits (n=1/immunization group) were administered their respective vaccine subcutaneously in a total volume of 0.5 mL without exogenous adjuvant. Qβ-Ct VD4 rabbits received 2 immunizations, three weeks apart and Qβ-Cm VD4 rabbits received 3 immunizations, three weeks apart. Sera was collected from rabbits 3 weeks after each immunization via the marginal ear vein to assess IgG antibody titers. 3-weeks after the final immunization, terminal sera was collected via cardiac puncture for in vitro assays.

### Quantification of Antibody Titers to Cognate Synthetic Peptides

All incubations occurred at 25° C. with shaking, washes were performed three times in 1×PBS, and volumes utilized were 50 μL, unless otherwise noted. Immulon 2 High Binding 96-well flat-bottom plates (Thermo Scientific) were coated with 0.5 jag of streptavidin (Invitrogen) and incubated overnight at 4° C. Plates were washed and 1 μg of Succinimidyl 6-(beta-maleimidopropionamido)-hexanoate (SMPH) was added for 1 hour. Plates were washed and incubated for 2 hours with 1 μg of synthetic peptide (Genscript). Plates were then washed and 150 μL of blocking solution (0.5% dry milk in 1×PBS) was added to each well and incubation continued overnight at 4° C. Plates were washed twice and 4-fold serial dilutions of sera or 2-fold serial dilutions of vaginal washes in blocking solution was added for 2 hours. Plates were washed five times and reacted with a 1:5000 dilution of peroxidase-conjugated AffiniPure goat anti-rabbit IgG secondary (Jackson ImmunoResearch) or peroxidase-conjugated AffiniPure goat anti-mouse IgG second (Jackson ImmunoResearch) in blocking solution, depending on the host sera. Incubation continued for 45 minutes before plates were again washed five times. Plates were reacted with 3,3′,5,5′-Tetramethylbenzidine (TMB, EMD Millipore) for 15 minutes, followed by quenching with 1% hydrochloric acid. Absorbance was read at 450 nm via accuSkan FC (Fisher Scientific). Background absorbances were averaged and values were subtracted from experimental wells. For¹¹⁻¹⁵ ELISAs measuring avidity, the protocol only differs by the addition of either distilled water or 4.8-6 M urea for a 10 minute incubation and additional washes before reaction with TMB. Binding of a broadly neutralizing monoclonal antibody against Chlamydia trachomatis was assessed as described using a mouse monoclonal.

### Propagation of CtsvD Elementary Bodies

CtsvD utilized for the murine challenge models were propagated and purified at the University of New Mexico as previously described^{24,55}. Ct serovars utilized for EB ELISAs were a generous gift from Dr. Harlan Caldwell. Briefly, CtsvD (strain UW-3-Cx, ATCC VR-885) was expanded in a series of 6-well flat-bottom tissue culture plates containing confluent McCoy cells (ATCC), at which point a single 6-well infected plate was harvested for infection of 6 T225 cm² tissue culture flasks. The frozen CtsvD infected 6-well plate was thawed in a 37° C. water bath. Cells were lysed via mechanical disruption and sonication to release CtsvD EBs. Cell debris was removed from supernatants via centrifugation. Supernatants were then utilized to infect 6 T225 cm² cell culture flasks containing confluent McCoy cells that were pretreated with 1×DEAE-Dextran and washed with Hanks' Balanced Salt Solution. Initial infection was allowed to continue for 2 hours at 37° C. with 5% CO₂. After 2 hours, Minimum Essential Medium (Gibco) containing 10% heat-inactivated fetal bovine serum (Gibco) and 1 μg/mL of cycloheximide (Sigma) was added to the infectious inoculum. Infection continued for an additional 42 hours. Flasks were then snap frozen at −80° C. until purification.

### Purification of CtsvD Elementary Bodies

6-12 frozen T225 cm² flasks containing infected McCoy cell monolayers were thawed in a 37° C. water bath for CtsvD EB purification. Sterile glass beads were added to each flask and mechanical disruption of monolayers was completed. Lysates were collected and underwent sonication at 30% power to further disrupt cells. Cell debris was removed via centrifugation and supernatants were transferred to Oak Ridge tubes. CtsvD bacteria was pelleted via ultracentrifugation at 29,500×g for 45 minutes. To separate EBs from reticulate bodies (RBs), the bacterial pellet was resuspended in SPG and underwent further sonication. Bacterial resuspension was overlayed onto 30% Gastrogafin density media (Bracco Diagnostics) and subjected to ultracentrifugation for 35 minutes. The EB pellet was resuspended into a small volume of SPG and sonicated once more. Sterility was confirmed via lack of bacterial growth in culture media and aliquoted CtsvD EBs were tittered and stored until further use at −80° C.

### Antibody Binding to Ct EBs via EB ELISA

Elementary body ELISAs were performed as previously described⁶⁷. Ct serovars utilized were: A2497, B/TW-5/OT, Ba/Ap-2, C/TW-3/OT, D/UW-3/cx, E/BOUR, F/IC-CA, G/uw-524/cx, H/UW-4/cx, I/UW-12UR, J/UW-36, K/UW-31, L1, L2, L3/404, Cm, kindly provided by Dr. Harlan Caldwell. All washes were performed three times using 1×PBS with 0.05% Tween-20 (PBST, Biorad). Briefly, Immulon 2 High Binding 96-well flat-bottom plates (Thermo Scientific) were coated with 1 μg/mL of poly-L-lysine (Sigma) in 0.05 M bicarbonate buffer. Plates were incubated overnight at 4° C. Plates were washed and EBs were added at 4.5 μg/mL in 1×PBS, as determined by a bicinchoninic acid protein assay (Pierce Thermo Scientific). Plates were then centrifuged for 5 minutes at 900×g before EBs were fixed via addition of 0.1% glutaraldehyde (Sigma) in 1×PBS. Plates were incubated for 20 minutes. Plates were washed and incubated in blocking solution (2% goat serum-PBST, Equitech Bio, Inc.) overnight at 4° C. 2-fold serum dilutions in blocking solution were added to each well after decanting blocking solution. Murine sera was pooled due to insufficient quantity. Incubation with sera continued for 2 hours with shaking. Plates were washed 4 times before addition of peroxidase-conjugated AffiniPure goat anti-rabbit IgG secondary (Jackson ImmunoResearch) or peroxidase-conjugated AffiniPure goat anti-mouse IgG second (Jackson ImmunoResearch) in blocking solution at a 1:5000 dilution, depending on the host sera. Incubation continued for 30 minutes with shaking before plates were washed again 4 times. Plates were then reacted with TMB for 20 minutes before the react^{3,22} ion was quenched with 1% hydrochloric acid. Absorbance was measured at 450 nm and background average absorbances were subtracted from experimental values. EB ELISAs using rabbit sera was done in multiple technical replicates.

### C57BL/6 Immunization Schedule and Chlamydia trachomatis Challenge

Female C57BL/6 mice (6-weeks, n=20/group) were administered 25 μg of Ct Qβ-VD4 or 5 μg of unconjugated Qβ in the hindleg intramuscularly three times, three weeks apart. Sera was collected via retroorbital eye bleed after each immunization to assess IgG antibody titers via peptide ELISA. Mice were administered 2.5 mg of medroxyprogesterone acetate (Amphastar Pharmaceuticals, Inc.) subcutaneously at days 10 and 3 pre-infection. Mice were then administered isoflurane anesthesia and 10⁵ IFU of CtsvD in 10 ΞL volume was instilled into the uterus of the mouse using a Non-Surgical Embryo Transfer Device (ParaTechs)^{24,68}.

### Quantification of Upper Genital Tract Bacterial Burden

At day 3 post-infection, 10 mice/group (Ct Qβ-VD4 and Qβ) were sacrificed via CO₂ administration and cervical dislocation before removal of the upper genital tracts. Upper genital tracts were stored in 1 mL of 1×PBS. Upper genital tracts were homogenized (VWR International). 100 μL of homogenate was utilized for DNA isolation via Qiagen DNeasy Blood & Tissue Kit following manufacturer's instructions. Isolated DNA was stored at −80° C. until use. Quantitation was performed via quantitative PCR as previously described⁵⁵. Custom 16s primer pairs were utilized from Integrated DNA Technologies (16sF 5′-GGA GGC TGC AGT CGA GAA TCT-3′, SEQIDNO:80; 16sR 5′-TTA CAA CCC TAG AGC CTT CAT CAC A-3′, SEQIDNO:81; and 16s Probe 5′-/56-FAM/TCG TCA GAC TTC CGT CCA TTG CGA/36-TAMSp/-3′, SEQ ID NO:82), along with a Rodent GAPDH kit (Applied Biosystems). Control samples included uninfected upper genital tract tissue DNA and CtsvD DNA. Controls were run alongside experimental samples to create a standard curve. qPCR reactions were completed in duplicate and in total volumes of 25 μL, with 10 μL of genomic DNA. Cycling conditions were as follows: 50° C. for 120 seconds, 95° C. for 600 seconds, 2 step amplification for 40 cycles at 95° C. for 15 seconds and 60° C. for 60 seconds. A standard curve was created using control sample values and experimental samples were normalized via GAPDH values before comparison to the CtsvD standard curve to obtain quantification of bacterial burden.

### Upper Genital Tract Pathology Quantification

At day 60 post-infection 10 mice/group (Ct Qβ-VD4 and Qβ) were sacrificed via CO2 administration and cervical dislocation before removal of the upper genital tracts. Gross pathology photos were acquired directly after removal of the organ. Upper genital tracts were placed into cassettes and stored in 10% normal buffer formalin for 24-48 hours before long-term storage in 70% ethanol. Gross pathology photos were again acquired of the fixed tissues. With the assistance of the University of New Mexico Human Tissue Repository and Tissue Analysis Shared Resource, tissues were embedded, sectioned, and underwent hematoxylin and eosin staining. Gross pathology photos were again acquired of the stained slides. Uterine horn dilation scores were acquired by counting the individual dilations within the horns of each upper genital tract. Three blinded scorers independently scored each tissue. Inconsistencies in scores were discussed among the blinded scorers until consensus was obtained. At this time, the tissues were unblinded. As additional controls, uterine horn dilation scores were also obtained from age matched naïve uninfected control mice (n=5) and age matched naïve CtsvD infected control mice (n=10).

### Balb/c Immunization Schedule and Luciferase-Expressing Chlamydia muridarum Challenge

Female Balb/c mice (6-weeks, n=10/group) were administered 5 μg of Cm Qβ-VD4 or 5 μg of unconjugated Qβ in the hindleg intramuscularly three times, three weeks apart. Sera was collected via retroorbital eye bleed before vaginal infection to assess IgG antibody titers via peptide ELISA. Mice were administered 2.5 mg of medroxyprogesterone acetate (Amphastar Pharmaceuticals, Inc.) subcutaneously at day 7 pre-infection. Mice were then administered isoflurane anesthesia and 2×10⁴ IFU of Luciferase-expressing Chlamydia muridarum in 20 μL volume was instilled into the vaginal cavity of the mouse⁶¹. At days 3 through 6 post-infection, bacterial burden was determined via an in vivo imaging system (IVIS Spectrum, PerkinElmer). Briefly, 200 μL of a 40 mg/mL solution of D-Luciferin (PerkinElmer) in 1×PBS was injected intraperitoneally into the abdomen of the mice. After at least 25 minutes, mice were put under anesthesia using isoflurane and positioned in the IVIS machine. Images were acquired using a firefly probe with 1 minute exposure time. To determine average radiance, uniform regions of interest (ROI) were selected on the genital tract of the mice. ROIs were also placed on the chest cavity of control mice to determine background average radiance, which was averaged and subtracted from experimental genital tract values. Analysis occurred using Live Image 4.3.1 software.

### Balb/c In Vivo Neutralization Chlamydia muridarum Challenge

Female Balb/c mice (6-weeks, n=2/group) were administered 5 μg of Cm Q(3-VD4 or 5 μg of unconjugated Qβ in the hindleg intramuscularly three times, three weeks apart. Terminal sera was then collected via cardiac puncture. Peptide ELISAs were performed as previously described to ensure high-titer antibody responses. Terminal sera was stored at −80° C. until use. Naïve female Balb/c mice (7-weeks, n=10/group) were administered 2.5 mg of medroxyprogesterone acetate (Amphastar Pharmaceuticals, Inc.) subcutaneously at day 7 pre-infection. Mice were challenged with a 1:1 volume mixture of mixed terminal immune sera and Luciferase-expressing Cm in a total volume of 20 μL. Briefly, mixed terminal immune sera was heat inactivated via incubation at 56° C. for 30 minutes. Terminal immune sera was then mixed with Luciferase-expressing Cm for a final IFU of 2×10⁴ at a 1:1 dilution. Mixture was then incubated at 37° C. for 30 minutes with occasional hand mixing. Mice were then administered isoflurane anesthesia and 20 μL volumes of terminal sera Luciferase-expressing Cm was instilled into the vaginal cavity of the mouse. IVIS was performed as previously described on days 3 through 6 post-infection.

### Balb/c Antibody Longevity Sample Collection

Female Balb/c mice (6-weeks, n=5/group) were administered 25 μg of Ct Qβ-VD4, 5 lag of Cm Qβ-VD4, or 5 μg of unconjugated Qr3 in the hindleg intramuscularly three times, three weeks apart. Mice were followed for 39 weeks post third immunization, collecting both sera and vaginal washes at various time points. Briefly, mice were put under anesthesia using isoflurane and sera was collected via retro-orbital eye bleed. Vaginal washes were collected by inserting 30 μL of 1×PBS into the vaginal cavity of the mouse using a P20 hand pipette. PBS was collected and a total of three repeated washes were collected and combined. Vaginal washes underwent a 10 minute centrifugation to remove cell debris. Both sera and vaginal washes were stored at −80° C. until use.

### Statistics

All statistical analysis was performed using GraphPad Prism 9 for macOS. Individual tests performed are noted in figure legends.

### Results

### Engineering Qβ-VD4 VLPs

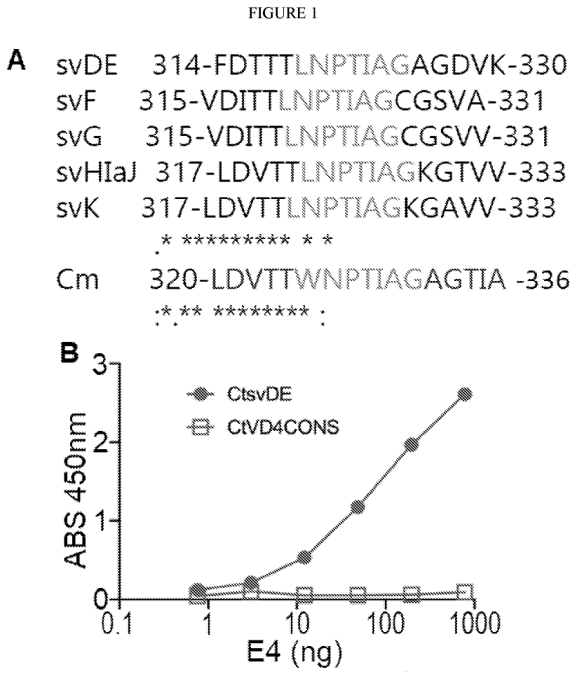

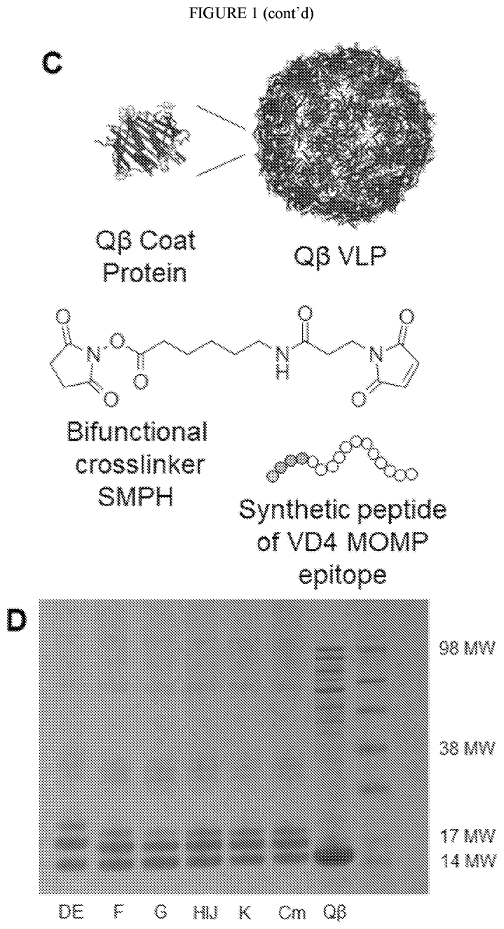

The core epitope of VD4 MOMP is an attractive vaccine target due to the amino acid sequence conservation among urogenital Ct serovars (FIG. 1A). Yet, it was found that the flanking amino acids of the conserved VD4 epitope are necessary for antibody recognition. Indeed, the E4 monoclonal, known to bind to the VD4 epitope and is cross-serovar neutralizing, only bound to an extended version of the epitope (FDTTTLNPTIAGAGDVK), instead of the conserved epitope alone (LNPTIAG) (FIG. 1B). Yet, the flanking regions of the conserved VD4 epitope are not perfectly conserved among urogenital Ct serovars (FIG. 1A). Therefore, to engineer a bacteriophage Qβ VLP vaccine that could elicit antibodies able to recognize all urogenital serovars, a mixed vaccine approach was undertaken. The Qβ VLP used is composed of 90 coat protein dimers, which contain surface-exposed lysines, that self-assemble into a VLP (FIG. 1C). These surface-exposed lysines are taken advantage of in order to chemically conjugate peptides of interest to the Qβ VLP using a bifunctional crosslinker, Succinimidyl 6-(beta-maleimidopropionamido)-hexanoate (SMPH) (FIG. 1C). In this case, the five modified synthetic peptides of interest are those representing all eight urogenital Ct serovars (FIG. 1A). Chemical conjugation of each peptide of interest to Qβ VLP was confirmed via SDS-PAGE gel (FIG. 1D). Once chemical conjugation was confirmed, a mixed vaccine was created by combining each of the five conjugated Qβ VLPs together in equal parts (Qβ-VD4). Separately, a Qβ VLP vaccine was created displaying the Chlamydia muridarum (Cm) sequence (Qβ-Cm VD4).

### Immunization with Qβ-VD4 Results in High-Titer Antibodies to Cognate Antigen Peptides

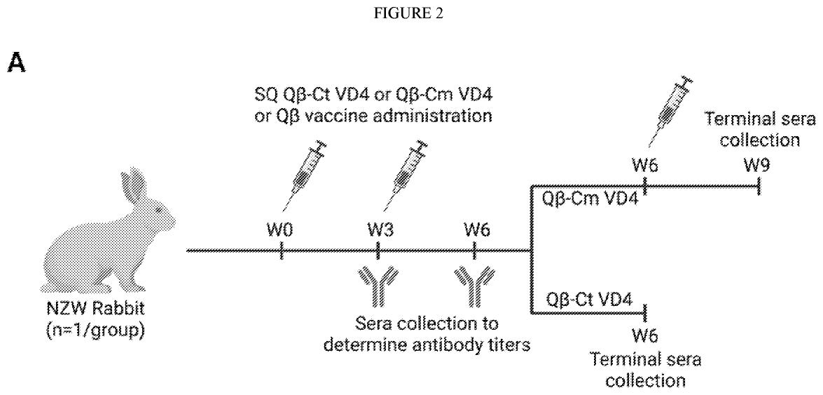

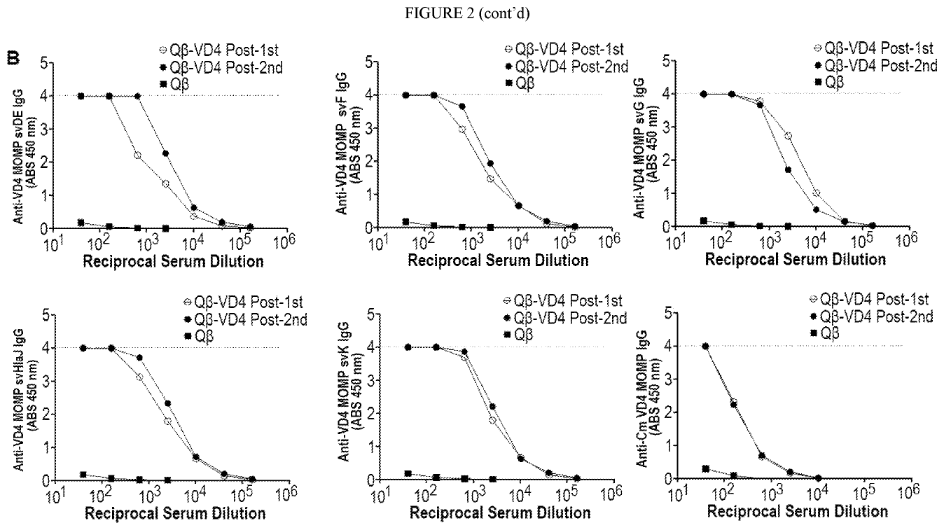

New Zealand White (NZW) rabbits (n=1/group) were administered the Qβ-VD4 vaccine subcutaneously twice, three weeks apart, before terminal immune sera was collected for investigation of in vitro antibody binding capacity (FIG. 2A). Immune sera IgG antibodies collected 3-weeks post the first and final immunization were investigated for binding potential to their cognate peptides. Immune sera IgG antibodies post the first and second immunization were able to recognize all five cognate antigen peptides via ELISA above that of terminal unconjugated Qβ control sera IgG antibodies (FIG. 2B). Further, in many cases, one immunization elicited similar antibody responses as two immunizations, such as that to serovar K peptide (FIG. 2B). In addition to a second dose not eliciting higher-titer antibodies, avidity indexes were also not significantly increased after administration of a booster (Figure Si).

### Qβ-VD4 VLP Immune Sera IgG Antibodies Binds to All Ct Serovars In Vitro

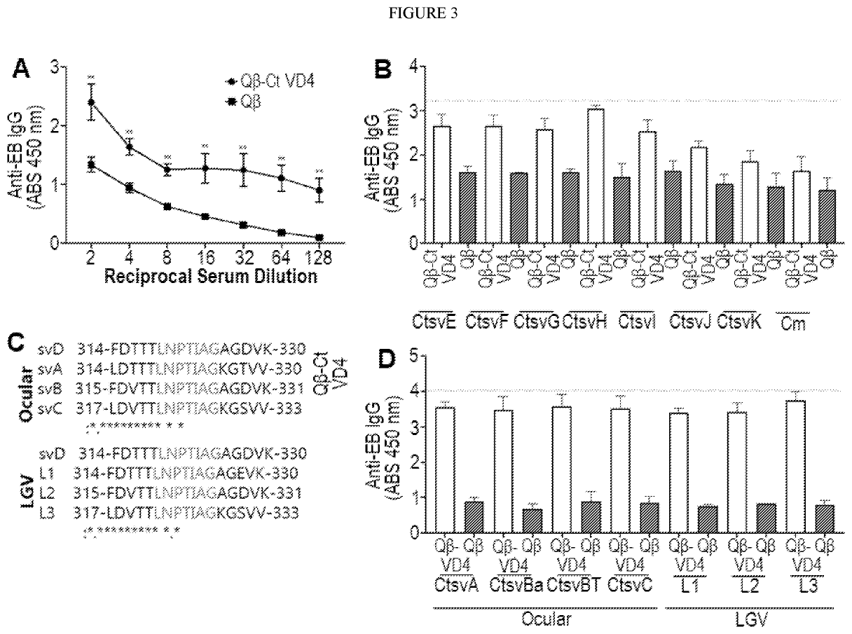

After finding that immune sera IgG antibodies recognized and bound to all urogenital Ct serovar cognate linear peptides (FIG. 2B), we investigated the terminal immune sera IgG antibodies for ability to bind to elementary body (EB) via ELISA. We found a statistically significant increase in binding of terminal sera IgG antibodies to CtsvD at all dilutions tested (1:2 through 1:128) above that of terminal unconjugated Qβ control sera IgG antibodies (FIG. 3A). Further, the terminal immune sera IgG antibodies was able to bind to all Ct urogenital serovars above that of unconjugated Qβ control sera IgG antibodies (FIG. 3B). Immune sera IgG was also able to bind to Cm, which has significant sequence homology to the urogenital serovars (FIG. 3B). Seeing that Qβ-VD4 immune sera IgG was able to bind to Cm, despite difference in flanking regions (FIG. 1A), we investigated binding capacity of terminal immune sera IgG to ocular trachoma Ct serovars (A-C) and lymphogranuloma venereum (LGV) Ct servoars (L1-3). Ocular and LGV Ct serovars also tend to have the highly conserved VD4 epitope (FIG. 3C). We found that Qβ-VD4 terminal immune sera IgG was also able to bind to both ocular and LGV Ct serovars above that of unconjugated Qβ control sera IgG (FIG. 3D).

### Qβ-VD4 VLP Vaccination Does Not Reduce Bacterial Burden or Pathology in an Intrauterine Ct Infection Mouse Model

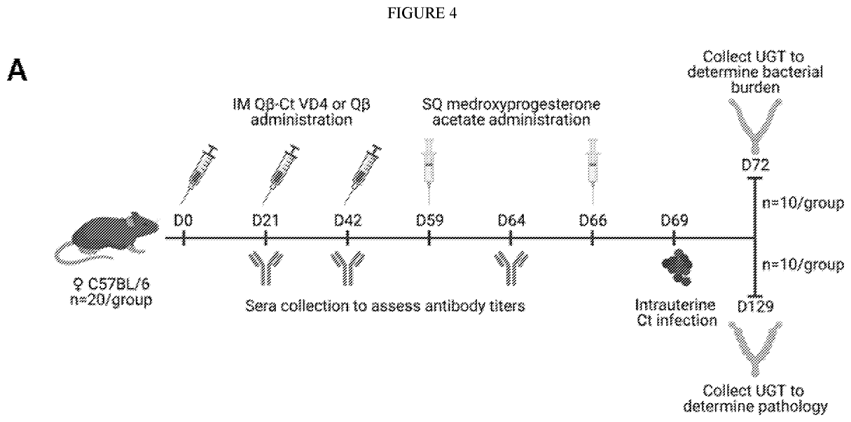

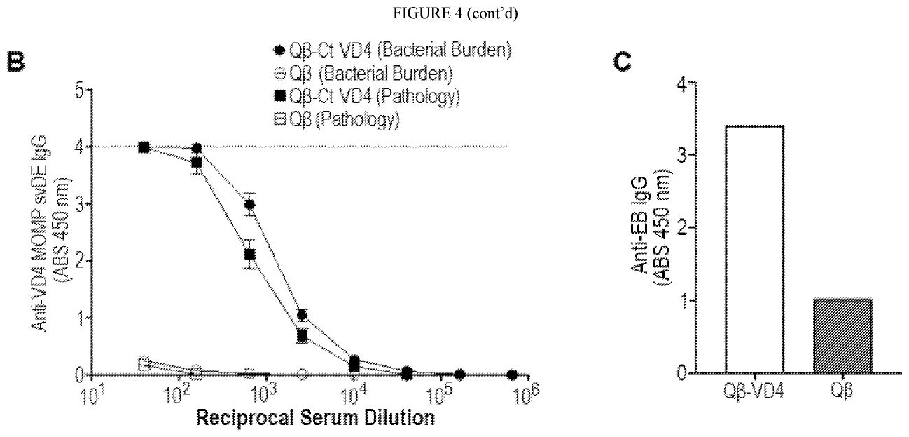

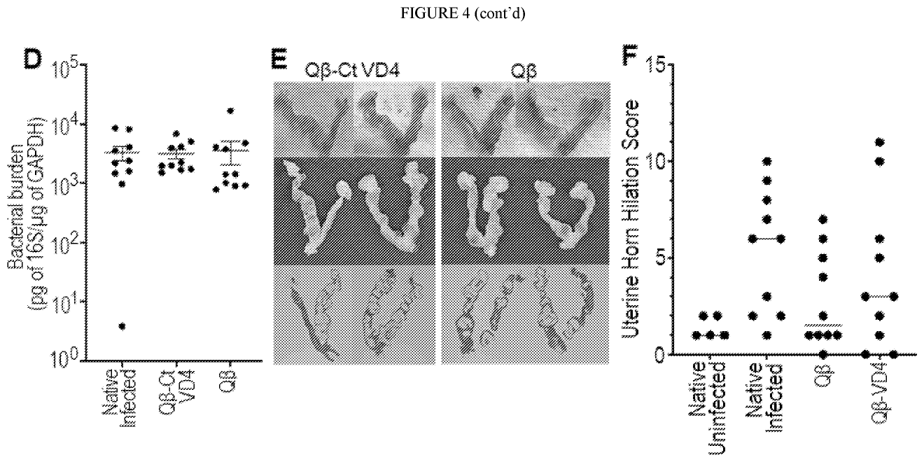

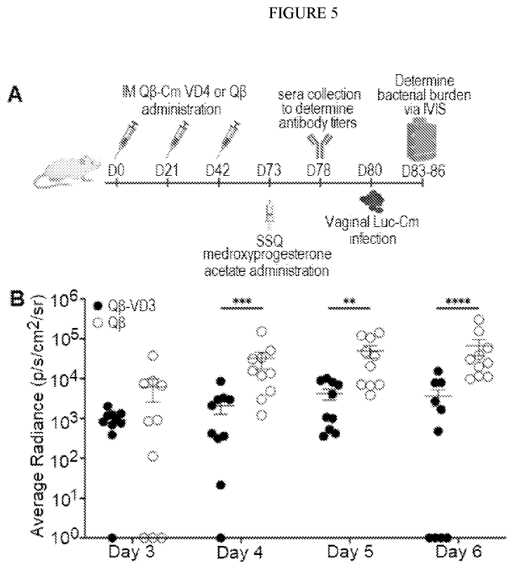

Having demonstrated that immune sera IgG had binding capacity for both the linear cognate peptides and Ct EBs (FIG. 2B, 3A-B), vaccine efficacy in a Ct murine challenge model was next investigated. Female C57BL/6 mice (n=10/group) were vaccinated with Ct Qβ-VD4 or unconjugated Qβ control vaccine three times, three weeks apart (FIG. 4A). Mice were administered medroxyprogesterone acetate at days 10 and 3 pre-infection and sera IgG antibody titers were determined after each immunization (FIG. 4A). As seen with immunization of NZW rabbits, high-titer peptide-specific IgG antibody responses were elicited via vaccination with Qβ-VD4 (FIG. 4B). Further, pooled murine immune sera IgG was able to bind to Ct serovar D via EB ELISA (FIG. 4C). Four weeks after the final immunization, mice were challenged with Ct serovar D via intrauterine instillation, bypassing the cervix, which is the initial site of infection for natural transmissions⁵⁷⁻⁶⁰. We found that at day 3 post-infection, there was not a statistical difference in bacterial burden in the upper reproductive tract of the mice, as measured by quantitative PCR, between the vaccinated and control mice (FIG. 4D). Further, at sixty days post-infection, pathology of the upper genital tract was assessed, which revealed no statistical difference in uterine horn dilations or gross visual pathology between vaccinated and control groups (FIG. 4E-F, Figures S2 and S3).

### Qβ-VD4 VLP Vaccination Protects Against Chlamydia muridarum Infection in Mice

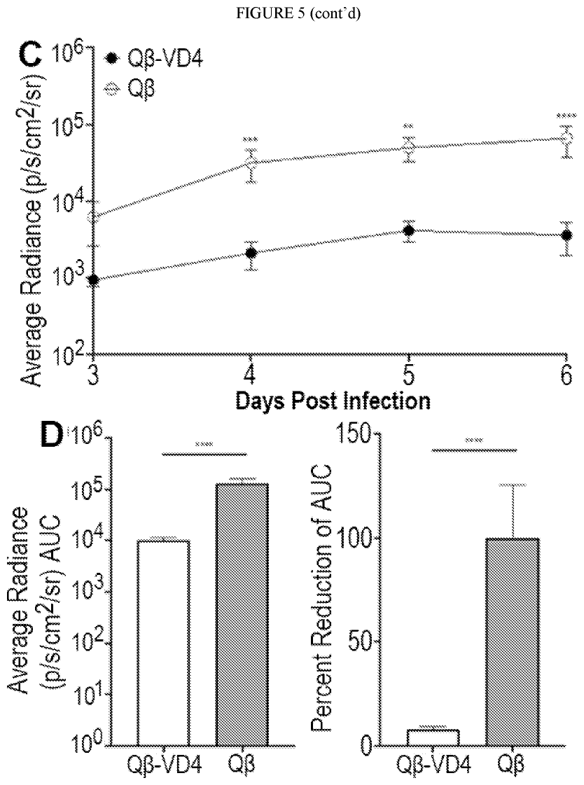

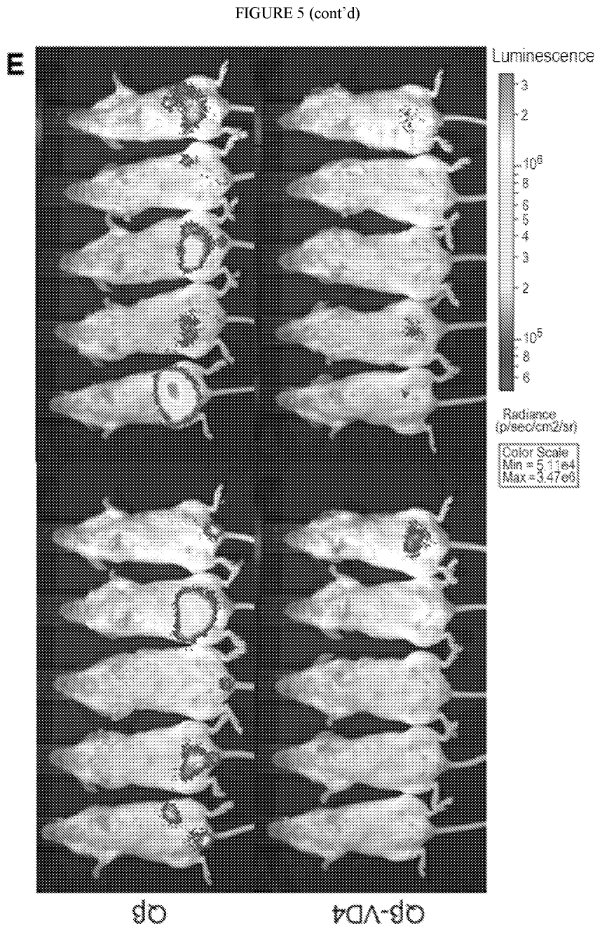

Having identified that Qβ-VD4 vaccination did not prevent establishment of Ct infection in the uterus, we next investigated the ability of Qβ-VD4 to prevent infection in a vaginal challenge, which is the natural route of transmission in humans. We used the Cm vaginal challenge model for these studies, utilizing a luciferase-expressing Cm strain (Luc-Cm) in order to track infection in live animals over time⁶¹. Since there are several amino acid differences in the vaccine target between Ct and Cm (FIG. 1A), we performed these studies using the Qβ-Cm VD4 vaccine. We immunized female Balb/c mice (n=10/group) with three doses of Qβ-Cm VD4, three weeks apart. Mice were administered medroxyprogesterone acetate and sera IgG antibody titers were determined one week before vaginal challenge with Luc-Cm at five weeks post-immunization (FIG. 5A). Qβ-Cm VD4 elicited high-titer serum IgG antibodies that recognized the cognate antigen peptide (LDVTTWNPTIAGAGTIA, SEQIDNO:31) (Figure S4A). Infection progression was monitored via an in vivo imaging system (IVIS) from day 3 post-infection through day 6 post-infection. At day 3 post-infection, the mean burden (as measured by average radiance) was similar between the Qβ-Cm VD4 vaccinated and Qβ vaccinated groups, though trending lower in the vaccinated group (FIG. 5B-C). By day 4 post-infection there was significantly less bacterial burden in the Qβ-Cm VD4 vaccinated group (FIG. 5B-C). This trend continued through day 6 post-infection (FIG. 5B-C). At this point, the Qβ-Cm VD4 vaccinated group had a 1.27 log decrease in mean bacterial burden, compared to the Qβ vaccinated group (FIG. 5B-C). The consistent decrease in bacterial burden over the course of infection is reflected by a statistically significant decrease in area under the curve and percent reduction of area under the curve (FIG. 5D). Together, the decrease in bacterial burden can be highlighted in the day 6 post-infection images of the mice, showing a striking difference between groups (FIG. 5E).

### Qβ-VD4 VLP-Induced Immunity is Mediated by Antibodies

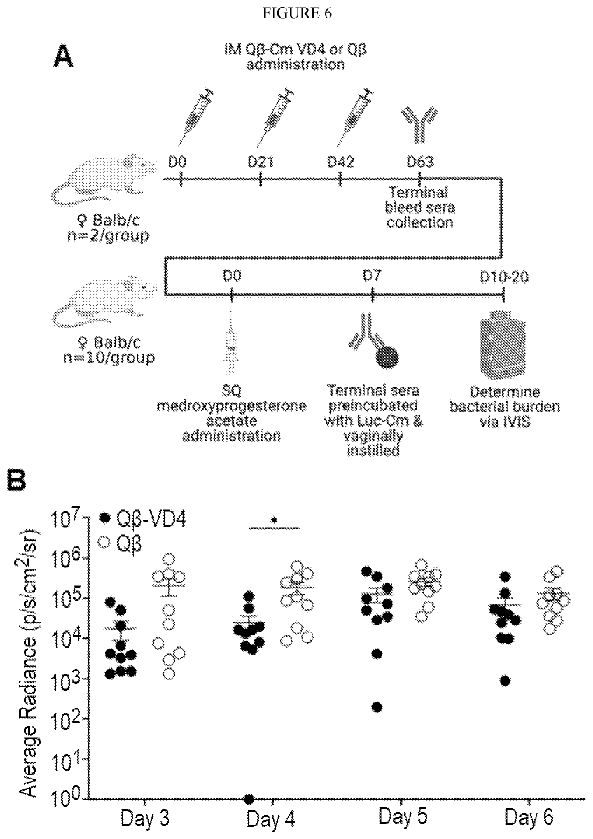

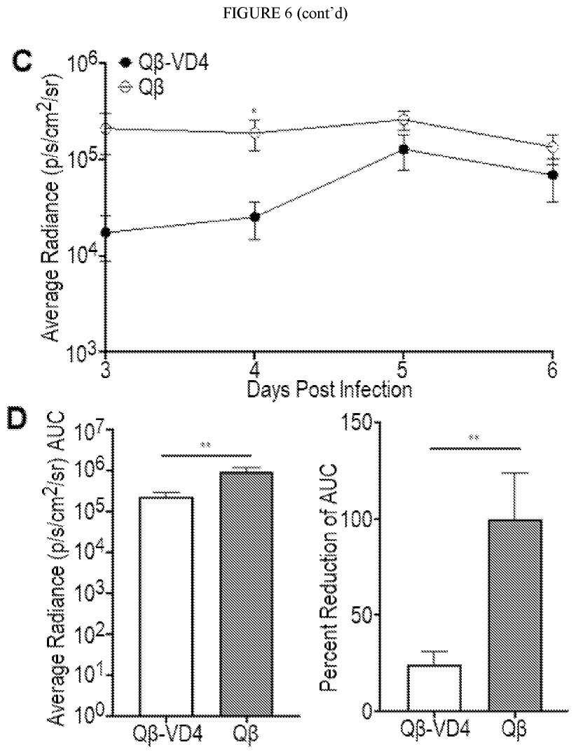

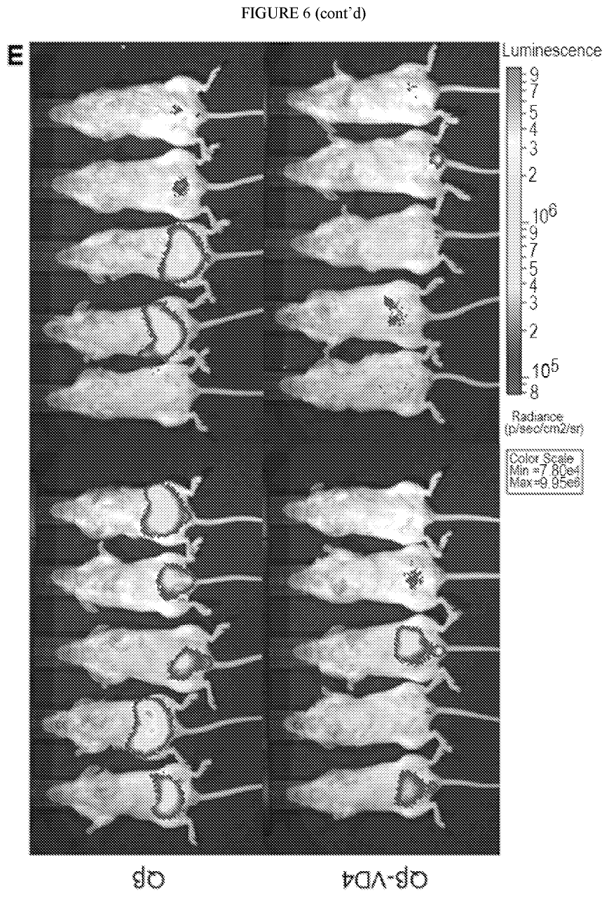

Having demonstrated efficacy of the Cm Qβ-VD4 VLP vaccine in a murine model, we next investigated if protection was mediated primarily via IgG antibodies, as has been shown previously of VLP-based vaccinations⁵⁶. We utilized an in vivo neutralization murine model, in which Cm is pre-incubated with heat-inactivated immune sera from Qβ-VD4 vaccinated mice or unconjugated Qβ vaccinated control mice before being instilled into the vagina of naive female mice Balb/c⁵⁵ (FIG. 6A). Bacterial burden is then monitored via IVIS at days 3-6 post-infection (FIG. 6A). At day 3, although not statistically significant, there is a trend toward lower bacterial burden in naive mice receiving Luc-Cm pre-incubated with Qβ-VD4 immune sera (FIG. 6B-C). By day 4, there is a significant difference in bacterial burden between the two groups (FIG. 6B-C). This resulted in a 0.87 log reduction in mean bacterial burden, compared to naive mice receiving Luc-Cm pre-incubated with Qβ immune sera. However, by days 5 and 6 post-infection, the mean bacterial burden is comparable between groups (FIG. 6B-C). However, the difference in mean bacterial burden seen at early post-infection time points is demonstrated by a significant reduction in area of under the curve and percent reduction of area under the curve (FIG. 6D). Together, the reduction in bacterial burden is highlighted by day 4 post-infection images of the mice (FIG. 6E).

### Qβ-VD4 VLP Vaccination Induces Long-Lived, High-Titer Sera IgG Antibodies

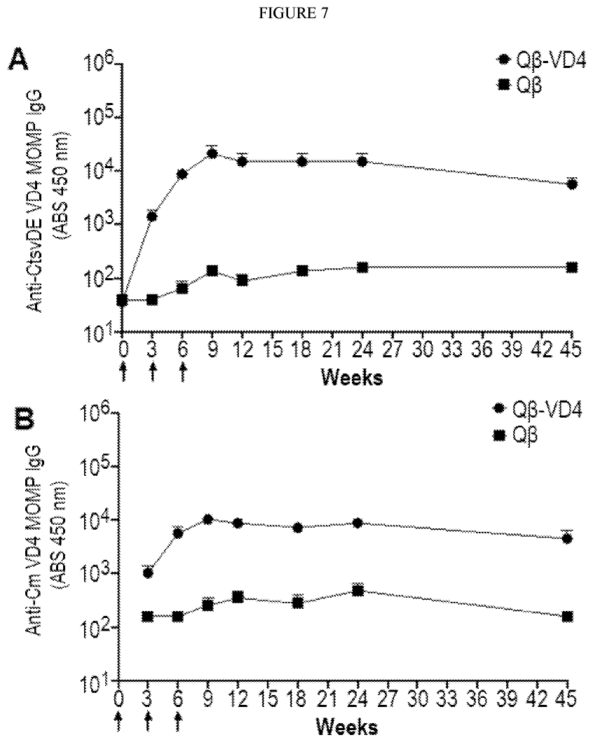

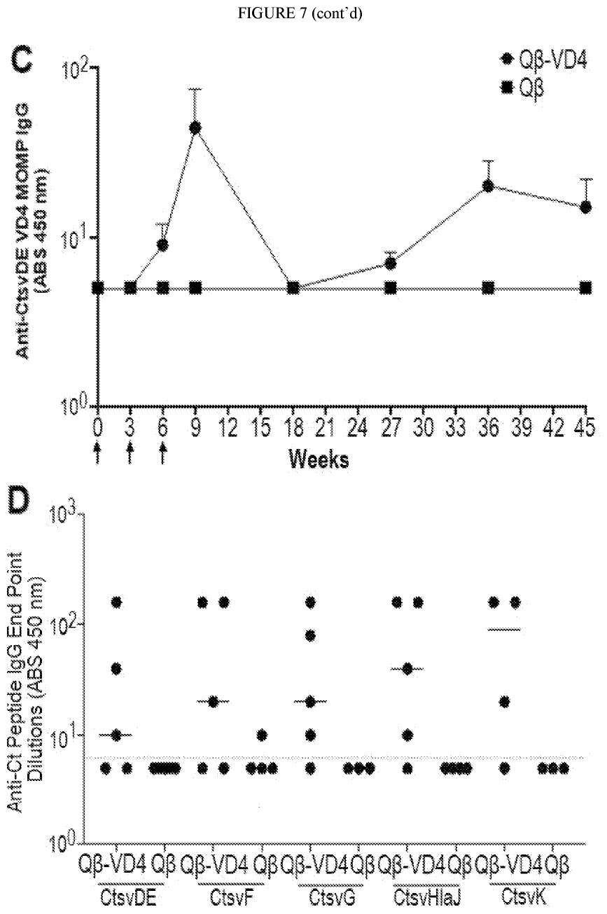

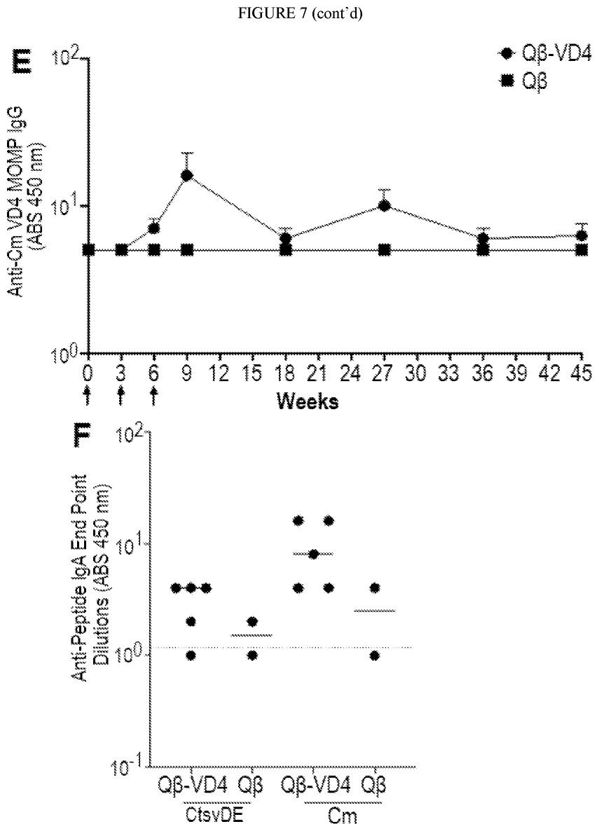

Since Qβ-VD4 vaccine-induced antibodies were shown to be important mediators of protection against vaginal Cm infection, we investigated the longevity of the antibody response. We vaccinated female Balb/c mice (n=5/group) three times, three weeks apart with either the Ct Qβ-VD4 vaccine, the Cm Qβ-VD4 vaccine, or the unconjugated Qβ control vaccine. We assessed the serum IgG antibody response and mucosal IgG and IgA antibody response over time via bait-peptide ELISA. We found that systemic IgG antibody response was long-lived in response to both the Cm and Ct Qβ-VD4 vaccines, lasting at least 39 weeks post the third immunization (FIG. 7A-B). Further, the IgG antibody response of the Ct Qβ-VD4 mixed vaccine continues to be long-lived, binding to all cognate peptides above that of Qβ immune sera through week 45 (Figure S5). Indeed, there is no significant decrease in antibody response over time. There were also high-titer peptide-specific IgG antibodies present in vaginal washes (FIG. 7C-E). Notably, the mucosal IgG antibody response was variable over time, likely as a result of sampling at different periods of murine estrous cycles (FIG. 7C, E). Additionally, it was found that mucosal IgG antibodies could bind to all peptides included in the mixed Ct Qβ-VD4 vaccine (FIG. 7D). IgA mucosal antibody responses were also present after vaccination, though at a lower dilution (FIG. 7F).

### DISCUSSION

A number of efforts have been undertaken to engineer a vaccine for urogenital Ct infection, and yet there is still no licensed vaccine. We, and others, have recognized MOMP as an attractive vaccine target^{32,33,49-52}. Indeed, the majority of antigen-based vaccines have targeted MOMP, recognizing MOMP as an important adhesion factor for Ct infection and as a target for neutralizing antibodies^{32,34,35,37,40-43,49,62} The only Ct vaccine (CTH522) to be tested in human Phase I clinical trials targeted MOMP³³. Although CTH522 has demonstrated safety and immunogenicity in women, it has not yet demonstrated efficacy in preventing infection or the long-term morbidity of Ct infection³³.

In this example, the inventors demonstrate antibody-mediated protection afforded by a novel Qβ-VD4 vaccine against urogenital Chlamydia infection in a vaginal murine infection model. The inventors found that Qβ-VD4 vaccination can elicit protection against vaginal Cm challenge, resulting in a 1.27 log bacterial burden reduction. The inventors identified that protection afforded by Qβ-VD4 vaccination is mediated primarily by antibodies, as demonstrated by an in vivo neutralization murine model. Indeed, pre-incubation of Luc-Cm with immune sera, which was then instilled into the vaginal cavity, resulted in a 0.87 log bacterial burden reduction. This is in agreement with previous studies, which demonstrated that passive transfer of immune sera to naive Rag1 knock-out mice could prevent vaginal Ct in approximately half of mice through day 21 post-infection³². Interestingly, the protection afforded by pre-incubation with immune sera did not match that seen in vaccinated mice and only lasted through day 4 post-infection. This is likely due to the fact that immune sera was not continuously present in the vagina of naive mice and all protective antibody was likely cleared by days 5 and 6 post-infection, allowing infection to proceed with additional antibody control.

The Qβ-VD4 vaccine has other features of that would be beneficial for a Ct vaccine, including long-lived antibody responses, potential cross-protection against all urogenital Ct serovars, and potential for a single dose vaccine. A Ct vaccine would likely require immune responses that last at least through the ages a woman is at most risk for Ct infection (15-24 years old), and those antibodies need to be at the site of infection to provide protection⁶³. Indeed, Qβ-VD4 elicits long-lived antibodies, lasting at least 39 weeks post the third immunization in murine models, and both IgG and IgA are detectable in the vaginal mucosa. Fluctuation in mucosal antibody titers was noted over time, which is likely due to sampling at different stages within the murine estrous cycles. Indeed, vaccine induced antibody titers can vary depending on stages of the estrous cycle, with higher IgA titers during estrus and higher IgG titers during diestrus⁶⁴. This has also been noted for women, where the entire immune environment can change throughout the female menstrual cycle, increasing the risk of STI and HIV transmission during certain stages of the menstrual cycle⁶⁵. We also demonstrate that high-titer antibodies are elicited in as few as one dose of vaccine, with little increase in titer after additional doses. Further, the quality of antibody response may not be further improved by additional doses, as the avidity index was not significantly increased after booster administration. Together, this suggests that a single dose of our vaccine may be sufficient to elicit protection, which would be a great advantage when considering global vaccine administration.

Qβ-VD4 elicits antibodies that bind to all urogenital Ct serovars, and ocular trachoma and LGV serovars, which suggests a cross-protection potential of this vaccine for all human Ct infections. The binding demonstrated here is likely afforded due to the amino acid conservation of the VD4 epitope, indicating its biological importance. Cross-serovar protection would be a notable advantage for Ct vaccination, since this has been a particular challenge for other Ct vaccine approaches. Indeed, CTH522 specifically targets Ct serovars D-G³³. Further investigations should be aimed at understanding protection that may be afforded against other Ct infections by Qβ-VD4.

Although Qβ-VD4 vaccine-elicited protection in mice from vaginal challenge was observed, protection was not observed when the inventors bypassed the cervix to instill Ct direction into the uterus. These data suggest that antibody-mediated protection against Ct infection is possible in the lower genital tract, but may no longer be protective once Ct ascends into the upper genital tract. However, there are likely important limitations to interpreting data generated from intrauterine challenge models. In this work, the inventors deposited 10⁵ IFU of CtsvD directly into the uterus. Yet, during natural infection the infectious dose that ascends into the uterus may be much lower. It is difficult to study the natural history of Ct infection and ascension in women, but mathematic modelling suggests that only 36% of Ct infections ascend to the upper genital tract⁶⁶. Further, more than 1000 bacteria ascend in only 9% of infections and more than 10,000 bacteria ascend in only 0.1% of infections⁶⁶. Together, this suggests that the infectious dose utilized in this work is unlikely to be biologically relevant (because the dose was too high) and future studies will be aimed at determining vaccine efficacy at lower infectious doses in the intrauterine challenge model. Further, because the natural route of transmission occurs in the lower female genital tract, and ascension is likely a consequence of persistent infection at the cervix, vaccines that aim to block or limit initial infection in lower genital tract and cervix should be tested in models that mimic this feature of infection.

### Example (OmcB Antigens) Second Set of References

In this example, the inventors hypothesized that targeting immunodominant B cell epitopes of Ct adhesion factors could mediate protection, as vaccine-induced antibodies may be at higher titers than antibodies elicited in response to natural infection. Indeed, IgG1 antibody responses toward OmcB in women with Ct infection are short-lived and not retained even after repeated Ct infections[19]. In previous work, the inventors identified the immunodominant B cell epitopes of 24 Ct antigens, including OmcB (amino acid 80-93, ATGPKQDSSFGRMY, SEQIDNO:83)[20].

OmcB (outer membrane complex B, CT443) is a surface-exposed protein characterized as a 60 kDa cysteine-rich polypeptide. Indeed, it is the second most abundant outer membrane protein. Its highly conserved nature among urogenital Ct serovars (D-K) suggests an important biological role for this protein. OmcB may, in fact, play a role in Ct's lifecycle via conversion from reticulate body (RB, metabolically active form) and elementary body (EB, infectious form) and contribute to cell wall rigidity[21,22]. Further, OmcB functions as an adhesion protein, binding to host glycosaminoglycans (GAGs)[23,24]. Indeed, recombinant OmcB expressed on the surface of E. coli is able to bind to both HeLa and Hec1B cells and this binding can be inhibited by the addition of anti-OmcB polyclonal serum[23]. Further, purified recombinant OmcB protein is able to inhibit 70% of Ct EB infectivity via in vitro assays[23]. Although epitopes mediating binding in Ct have not been fully elucidated, this provides a strong rational for developing vaccines that might elicit antibodies capable of blocking entry by OmcB.

Here, the primary aim was to engineer a vaccine targeting OmcB and assess the vaccine for immunogenicity and efficacy in relevant murine challenge models. The inventors previously identified a B-cell epitope of OmcB that was immunogenic during urogenital Ct infection in women[20]. The inventors demonstrate that our vaccine platform elicits high-titer IgG antibodies that recognize both the cognate linear peptide antigen and the epitope in its native conformation via EB ELISA. Further, immunization with our vaccine candidate reduces bacterial burden in a vaginal Chlamydia muridarum murine challenge model.

### Materials and Methods

### Production and Purification of Qβ VLPs

Bacteriophage QP VLPs were grown and purified as previously described[25,26]. Briefly, Escherichia coli C41 electrocompetent cells (Sigma) were transformed to include a Qβ coat protein containing plasmid, under Kanamycin antibiotic resistance. Subsequent E. coli was grown in lysogeny broth (LB), and the culture was induced once the optical density (OD₆₀₀) reached 0.6-0.8 using 0.5 mM Isopropyl β-D-1-thiogalactopyranoside (IPTG). Induction was allowed to continue for 3 hours before cells were pelleted and frozen at −80° C. Pellets were thawed and cells were chemically lysed via lysozyme solution for 1 hour at 4° C., followed by multiple rounds of sonication. 10 mg/mL DNase and 2 mM MgCl₂ were added, and incubation continued for 1 hour at 37° C. Cell debris was pelleted via centrifugation. Ammonium sulfate was added to supernatants to achieve 60% saturation before undergoing overnight incubation at 4° C. Mixtures underwent ultracentrifugation, with pellets resuspended. Centrifugation is repeated and supernatants containing Qβ are collected and frozen at −80° C. Isolation of Qβ occurs using size exclusion chromatography using a Sepharose column. Small volume fractions are collected with samples ran on a 1% agarose gel to confirm Qβ containing fractions. Further identification of Qβ containing fractions is confirmed via SDS-PAGE gel. Qβ containing fractions are combined, purified, and concentrated using 100 K Amicon Ultra Centrifugal Filters (Merk Millipore). Purified and concentrated Qβ concentration is determined via SDS-PAGE Gel using Hens Egg Lysozyme controls.

### Conjugation of Modified Peptides to the Surface of QβVLPs

Purified Qβ VLPs are incubated with a bifunctional crosslinker, Succinimidyl 6-(beta-maleimidopropionamido)-hexanoate (SMPH, ThermoScientific), for 2 hours at 25° C. with shaking. Excess SMPH is removed via 100 K Amico Ultra Centrifugal filtration before Qβ-SMPH is incubated with the peptide of interest at a 1:10 ratio. Epitopes of interest are modified to contain a terminal -GGGC linker sequence to facilitate conjugation when peptides are commercially manufactured (GenScript). Incubation of Q(3-SMPH with the peptide of interest continues overnight at 4° C. Unconjugated peptide is removed via 100 K Amicon Ultra Centrifugal filtration. Conjugation of peptide to Qβ coat protein is confirmed via a denaturing SDS-PAGE Gel. Conjugated Qβ VLPs are assumed to have undergone half loss due to filtration. Vaccines are properly diluted in 1×PBS for a final immunization of concentration of 25 μg/New Zealand White rabbit and 5 μg/Balb/c mouse. Vaccines are stored at −80° C. until use.

### Ethics Statement for Animal Studies

Animal studies were approved by the University of New Mexico School of Medicine Institutional Animal Care and Use Committee (19-200867). NZW rabbits were acquired from Charles River Laboratories and Balb/c mice were acquired from the Jackson Laboratory.

### Immunization of New Zealand White Rabbits

Female NZW rabbits (age 6 weeks) were allowed to acclimate for 3 weeks before experimentation procedures began. Rabbits (n=1/immunization group) were immunized with two doses of their respective vaccine, 3 weeks apart, subcutaneously in a total volume of 0.5 mL without exogenous adjuvant. Sera was collected from rabbits 3 weeks after the first dose via the marginal ear vein to assess IgG antibody titers. 3 weeks after the second dose, terminal sera was collected via cardiac puncture to assess immunogenicity.

### Peptide ELISA to Assess Vaccine Immunogenicity

Peptide ELISAs are performed as previously described[20,25]. Incubations are performed at 25° C. with shaking, washes are performed using 1×PBS three times, and 50 μL volumes are utilized, unless otherwise noted. Immulon 2 High Binding 96-well flat-bottom plates (Thermo Scientific) were coated with 0.5 lag of streptavidin (Invitrogen). Incubation continued overnight at 4° C. before plates were washed. 1 μg of SMPH (ThermoScientific) was added for 1 hour before plates were again washed and 1 μg of synthetic peptide (GenScript) was added for 2 hours. Plates were again washed and incubated with 150 μL of blocking solution (0.5% dry milk in 1×PBS) overnight at 4° C. Plates were washed and serial dilutions of sera in blocking solution was added for 2 hours of incubation. Plates were washed 5 times in 1×PBS before they were reacted with a 1:5000 dilution of peroxidase conjugated AffiniPure goat anti-rabbit or goat anti-mouse IgG secondary (Jackson ImmunoResearch) in blocking solution. Plates were washed again 5 times after a 45-minute incubation with secondary antibody. Plates were then reacted with 3,3′5,5′-Tetramethylbenzidine (TMB, EMD Millipore) for 15 minutes. Plates were then immediately quenched utilizing 1% hydrochloric acid (HCl). Absorbance values were read at 450 nm using accuSkan FC (Fisher Scientific). Background absorbance values were averaged and subtracted from experimental values.

### Growth and Purification of Chlamydia serovars

Chlamydia stocks utilized include Chlamydia trachomatis serovar D (strain UW-3-Cx, ATCC VR-885), Chlamydia muridarum (strain Nigg II), and Luciferase-expressing Chlamydia muridarum (kind gift of Dr. Guangming Zhong). Chlamydia was propagated as previously described[11,27,28]. Briefly, Chlamydia was expanded in a series of 6-well flat-bottom tissue culture plates containing confluent McCoy cells (ATCC). Once expanded, a single infected 6-well plate was harvested to infect 6 T225 cm² tissue culture flasks. Briefly, the frozen infected 6-well plate was thawed in a 37° C. water bath, at which point cells were lysed via mechanical disruption and sonication to release EBs. Cell debris was removed via centrifugation and supernatants were collected. 6 T225 cm² cell culture flasks containing confluent McCoy cells were pretreated with 1×DEAE-Dextran and washed with Hanks' Balanced Salt Solution (Gibco). Flasks were infected with collected supernatants containing EBs. Infection continued for 2 hours at 37° C. with 5% CO₂, with occasional gentle rocking of media. After 2 hours of incubation, Minimum Essential Medium (Gibco) with 10% heat-inactivated fetal bovine serum (Gibco) and 1 μg/mL of cycloheximide (Sigma) was added to the infectious inoculum and infection continued for 36-42 hours. At this point, flasks were snap frozen at −80° C. until purification of EBs. 6-12 frozen T225 cm² flasks containing infected McCoy cells were purified at once. Flasks were thawed in a 37° C. water bath before the addition of sterile glass beads to facilitate mechanical disruption. Lysates were collected and further lysed via sonication. Cell debris was removed via centrifugation. Supernatants were transferred to Oak Ridge tubes and bacteria was pelleted via ultracentrifugation at 29,500×g for 45 minutes. The bacterial pellet was resuspended and underwent further sonication. Separation of EBs and RBs was completed via overlaying the bacterial resuspension onto 30% Gastrogafin density media (Bracco Diagnostics) and ultracentrifugation for 35 minutes. The EB pellet was resuspended into a small volume and sonicated once more. EB aliquots were stored at −80° C. until further use. Sterility of prep was confirmed via lack of bacterial growth in culture media.

### Chlamydia Elementary Body ELISA

EB ELISAs were performed as previously described[29]. All washes were performed three times with 1×PBS with 0.05% Tween-20 (PBST). Immulon 2 High Binding 96-well flat-bottom plates were incubated with 1 μg/mL of poly-L-lysine (Sigma) in 0.05 M bicarbonate buffer overnight at 4° C. Protein concentrations of EBs were determined via bicinchoninic acid protein assay (Pierce Thermo Scientific). After plates were washed, EBs were added at approximately 22.5 μg/mL in 1×PBS. Plates underwent centrifugation at 900 ×g for 5 minutes before fixation with 0.1% glutaraldehyde (Sigma) in 1×PBS. Fixation was allowed to continue for 20 minutes before washing plates. Blocking solution (2% goat serum-PBST, Equitech Bio, Inc.) was added and incubation continued overnight at 4° C. Serial dilutions of serum in blocking solution was added after decanting blocking solution. Incubation with sera continued for 2 hours with shaking and plates were washed 4 times. Addition of a 1:5000 dilution of peroxidase conjugated AffiniPure goat anti-rabbit IgG secondary (Jackson ImmunoResearch) in blocking solution was added. Incubation continued for 30 minutes with shaking. Plates were washed 4 times and reacted with TMB for 20 minutes. The reaction was quenched with 1% HCl. Absorbance values were measured at 450 nm. Background values were averaged and subtracted from experimental values.

### Immunization of Balb/c Mice

Female Balb/c mice (aged 6-weeks, n=8-10/group) were administered 5 μg of conjugated Qβ vaccines or unconjugated Qβ (negative control) intramuscularly in the hindleg three times, three weeks apart. Sera was collected via retroorbital eye bleed after final immunization to determine IgG antibody titers via peptide ELISA.

### Luciferase-Expressing Chlamydia muridarum Vaginal Challenge

After immunization course was completed, mice were vaginally challenged with Chlamydia. 7 days before vaginal challenge, mice were administered 2.5 mg of medroxyprogesterone acetate (Amphastar Pharmaceuticals, Inc.) subcutaneously in 1× PBS. 2×10⁴ IFU of Luciferase-expressing Chlamydia muridarum in 20 μL volumes was instilled into the vaginal cavity of mice[30]. Bacterial burden was determined using an in vivo imaging system (IVIS Lumina II, PerkinElmer) at days 3-8 post-infection. This was completed by administering 200 μL of a 40 mg/mL solution of D-Luciferin (PerkinElmer) in 1×PBS intraperitoneally and imaging the mice at least 25 minutes afterwards. Images were acquired using a firefly probe with 1 minute exposure times.

### Determination of Bacterial Burden via IVIS

Bacterial burden was determined via average radiance. Uniform regions of interest (ROI) were selected on the genital tract of the mice and average radiance measurements were acquired. ROIs were also placed on the chest cavity of the control mice to determine background average radiance. Background average radiance of all control mice were averaged and subtracted from experimental genital tract values. All analysis occurred using Live Image 4.3.1 software.

### Statistical Analysis

Statistical analysis was performed utilizing GraphPad Prism 9 for macOS. Individual tests performed are noted in figure legends. *p≤0.05, **p≤0.01, ***p≤0.001, ****p≤0.0001.

### Results

### Engineering QβVLP Vaccines Targetting the Immunodominant B Cell Epitope of OmcB

In prior work, the inventors identified an immunodominant B cell epitope of OmcB as ATGPKQDSCFGRMY (amino acid 80-93, UniProt #P0CC04, SEQIDNO:83) by utilizing a novel technology called Deep Sequence-Coupled Biopanning and sera from women with a history of urogenital Ct infection[20]. In addition to being the most immunodominant B cell epitope of OmcB, the inventors also identified OmcB as the second most immunodominant

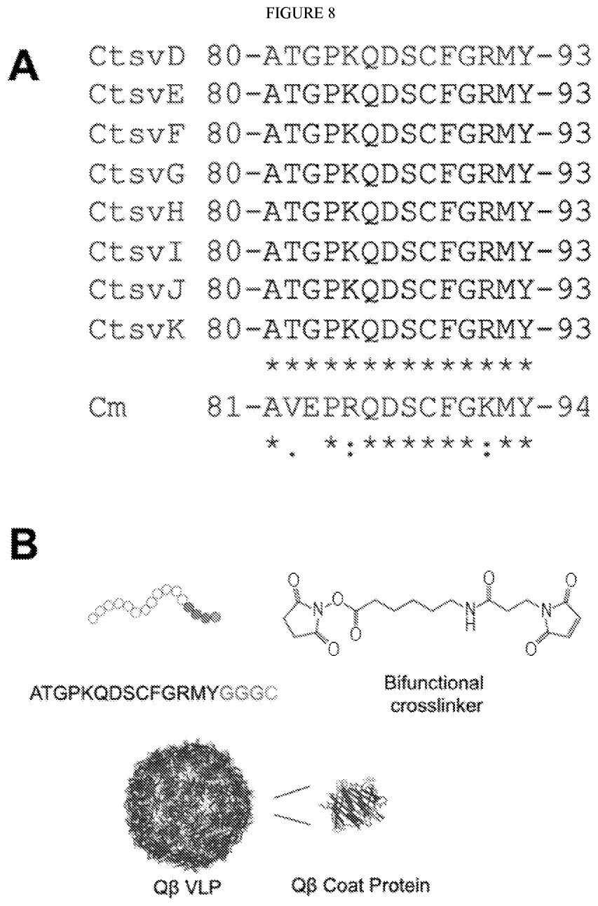

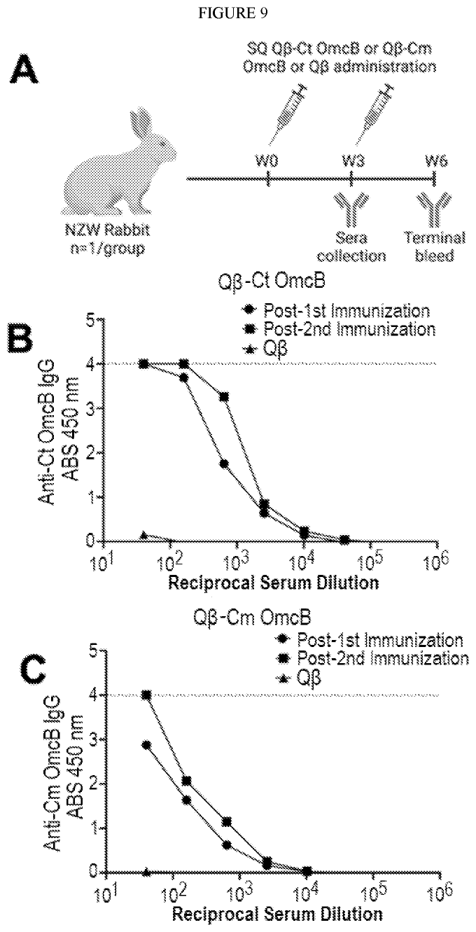

protein antigen that was included within our library [20]. The amino acid sequence for this epitope is perfectly conserved among urogenital Ct serovars D-K (FIG. 8A). Additionally, it is highly conserved between Cm and Ct (FIG. 9A). Accordingly, the inventors engineered vaccines targetting this region by chemically conjugating the peptide of interest to surface-exposed lysines on the coat protein of Qβ using a bifunctional crosslinker, SMPH (FIG. 8B). Notably, this was accomplished by modifying the epitope of interest during manufactuering to contain a C-terminal tri-glycine linker sequence, followed by a cysteine (FIG. 8B). We engineered two versions of our vaccine, utilizing both the Ct and Cm sequence.

### QβVLP Vaccines Targetting the Immunodominant Epitope of OmcB Elicit High-Titer Antibodies

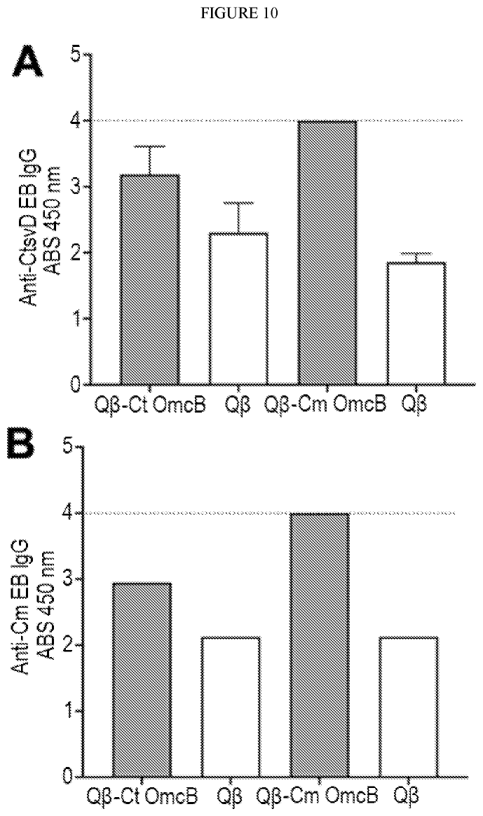

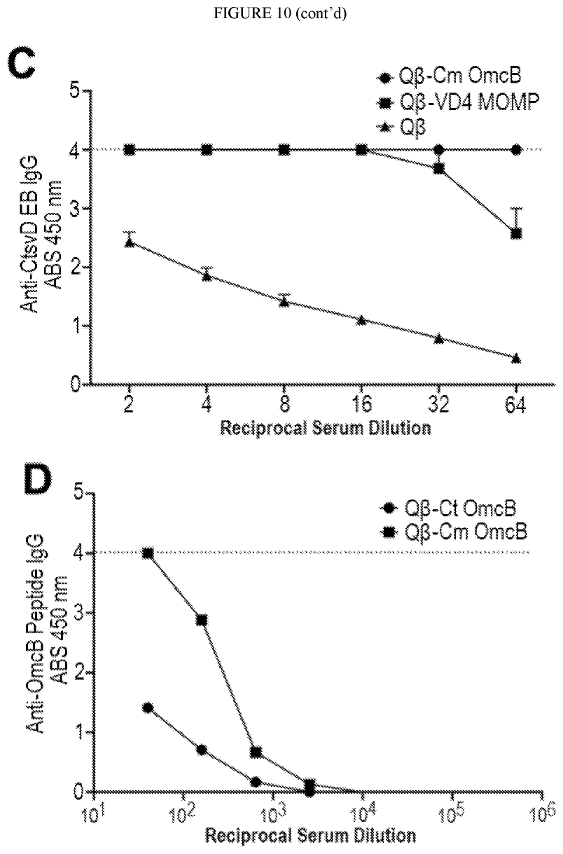

NZW rabbits (n=1/group) were immunized twice with their respective Qβ VLP vaccine, three weeks apart, or an unconjugated Qβ vaccine to serve as a negative control. Three weeks after the final immunization, terminal immune sera were collected to determine immunogencity via in vitro assays (FIG. 9A). Immune sera collected both 3-weeks after the first and second immunization were utilized to determine antibody binding to their cognate antigens via peptide ELISA. Both vaccine groups showed binding to their respective cognate peptides above that of control Qβ sera IgG (FIG. 9B-C). Additionally, both vaccine groups benefited from a vaccine boost 3-weeks post the first immunization, with higher absorbance values across a subset of reciprocal serum dilutions (FIG. 9B-C). Since immune sera IgG was able to bind to cognate peptide, the inventors investigated if terminal immune sera IgG could bind to Ct and/or Cm itself via an EB ELISA. The inventors found that Qβ-Ct OmcB serum IgG was able to bind to CtsvD above the Qβ control serum IgG (FIG. 10A). Further, despite amino acid sequence differences, the Qβ-Cm OmcB terminal sera IgG was also able to bind to CtsvD above Qβ (FIG. 10A). Likewise, both Qβ-Ct OmcB and Qβ-Cm OmcB terminal sera IgG bound to Cm above that of Qβ (FIG. 10B). Interestingly, we determined that Qβ-Cm OmcB terminal sera IgG may demonstrate higher absorbance values, and therefore better binding, to CtsvD than Qβ-Ct OmcB terminal sera IgG. Further investigated was the binding of Qβ-Cm OmcB terminal sera IgG to CtsvD across a range of serial dilutions, with the addition of a positive control, terminal sera from a NZW rabbit immunized with the VD4 epitope of MOMP. Strikingly, it was found that, despite OmcB being less abundant on the terminal sera IgG. Therefore, further investigated was the binding potential of Qβ-Cm OmcB terminal sera IgG to the Ct OmcB peptide and vice versa, surface CtsvD than MOMP[23], Qβ-Cm OmcB terminal sera had higher absorbance values than Qβ-VD4 sera and bound at the highest absorbance reading through a sera dilution of 1:64 (FIG. 10C). These results led us to investigate cross-binding potential of Qβ-Cm OmcB In agreement with the EB ELISA data, we found that Qβ-Cm OmcB terminal sera bound to Ct OmcB peptide, despite the amino acid differences in the sequence (FIG. 10D). Alternatively, the Qβ-Ct OmcB terminal sera IgG was largely unable to bind to the Cm OmcB peptide (FIG. 10D).

### Immunization with Qβ-Cm OmcB (C) Provides Moderate Protection Against Vaginal Chlamydia muridarum Infection

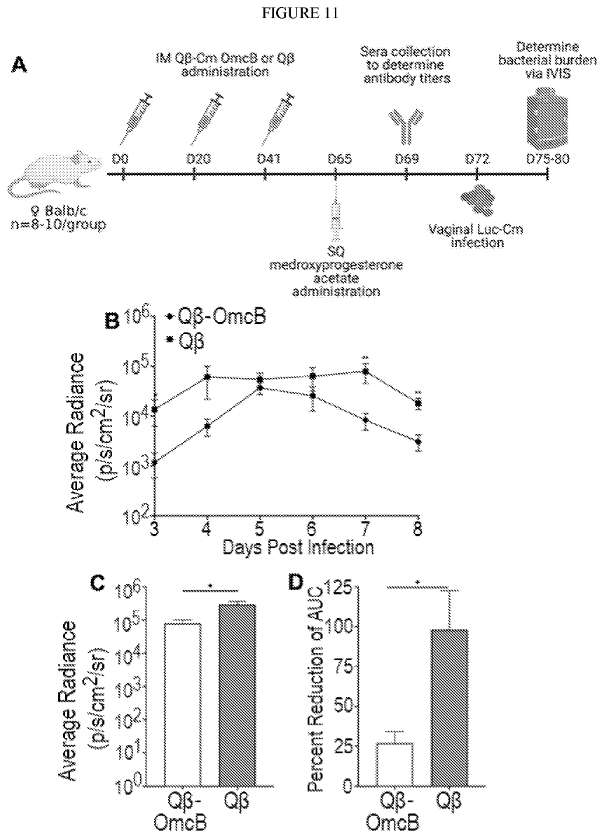

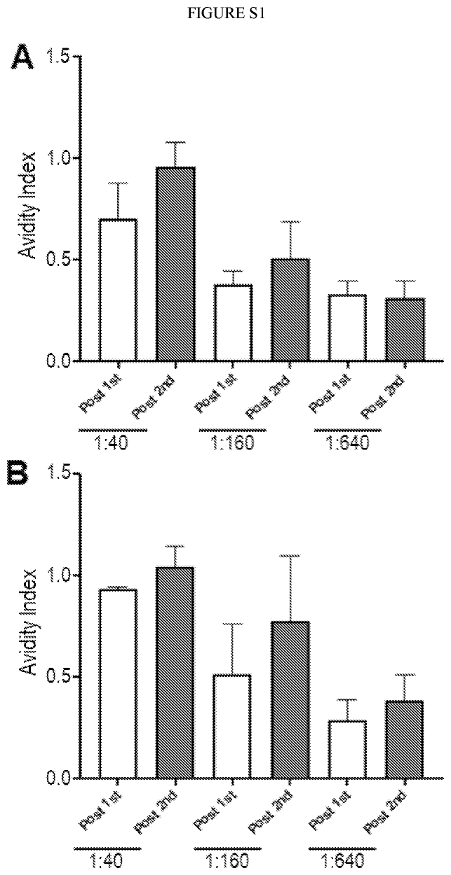

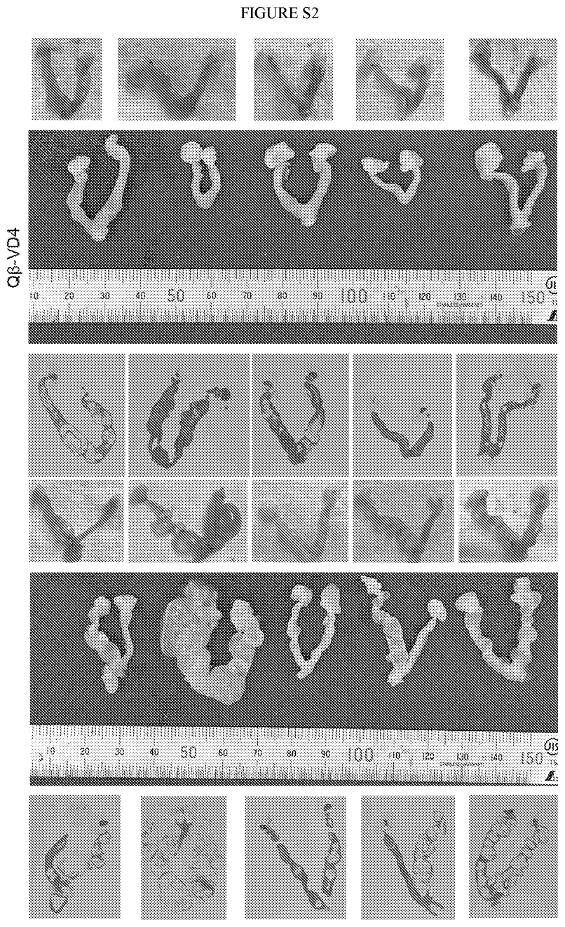

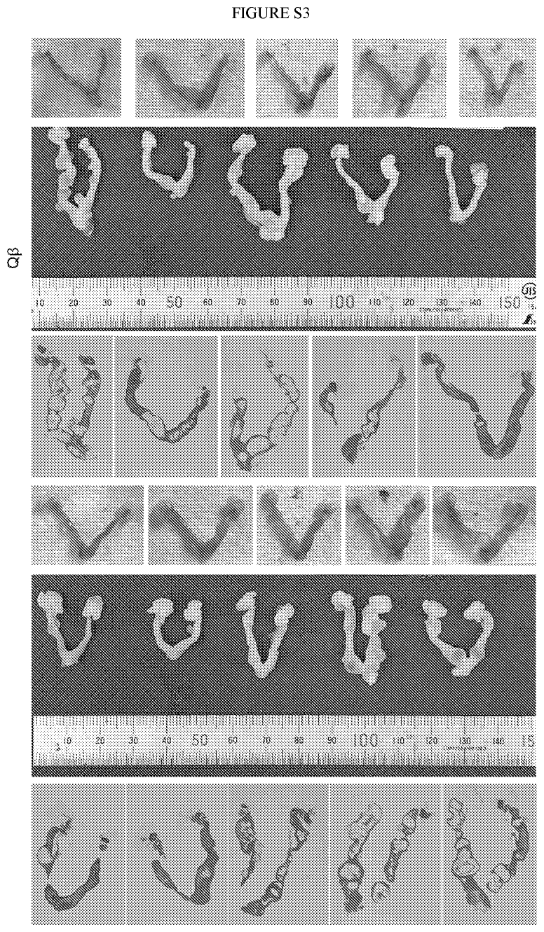

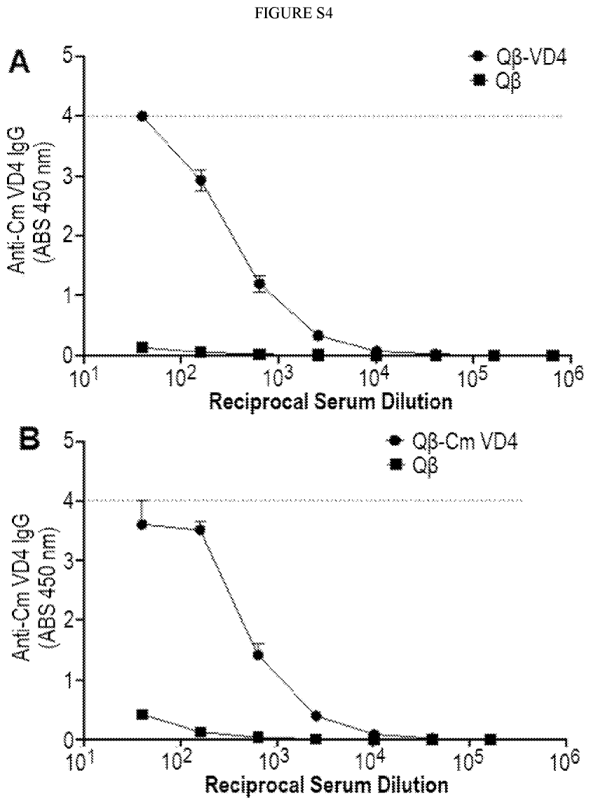

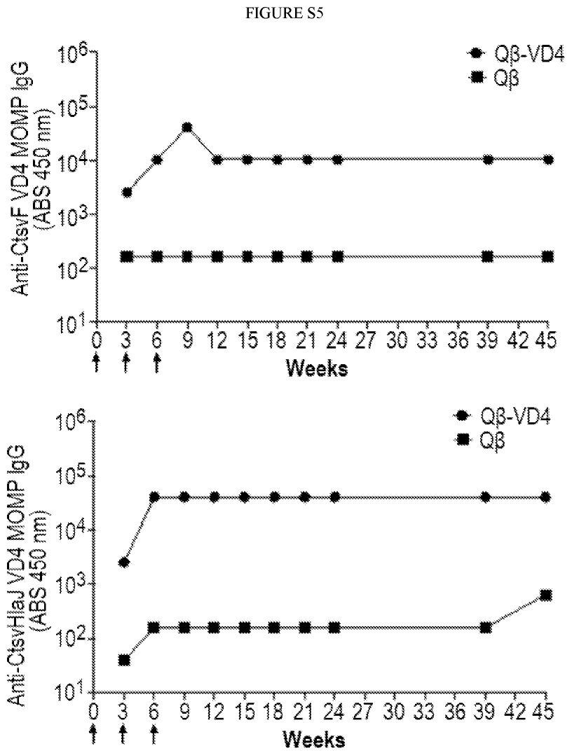

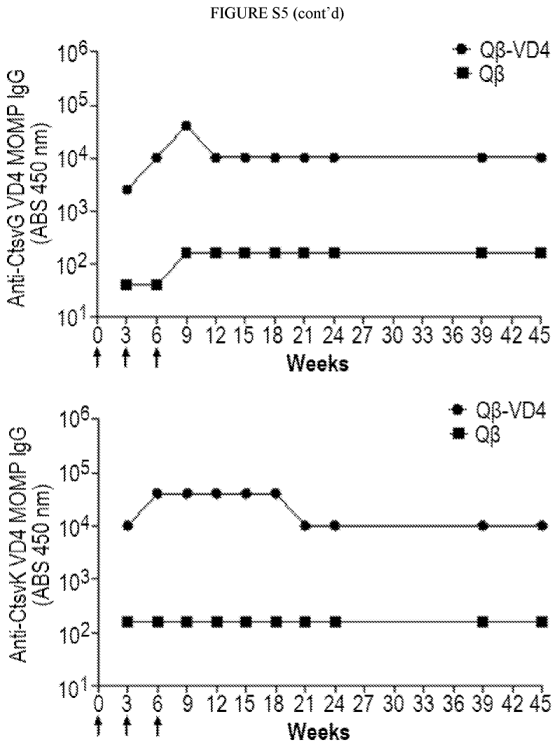

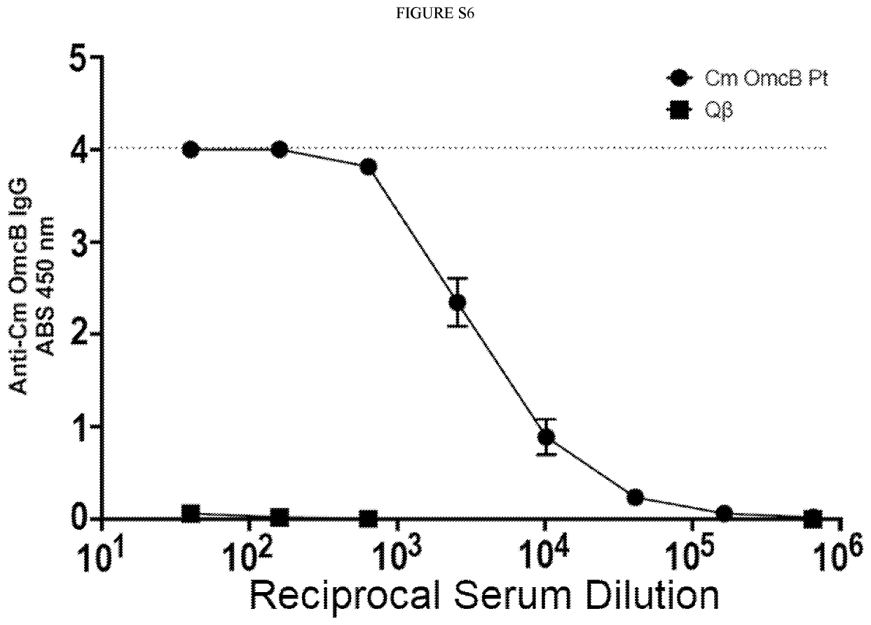

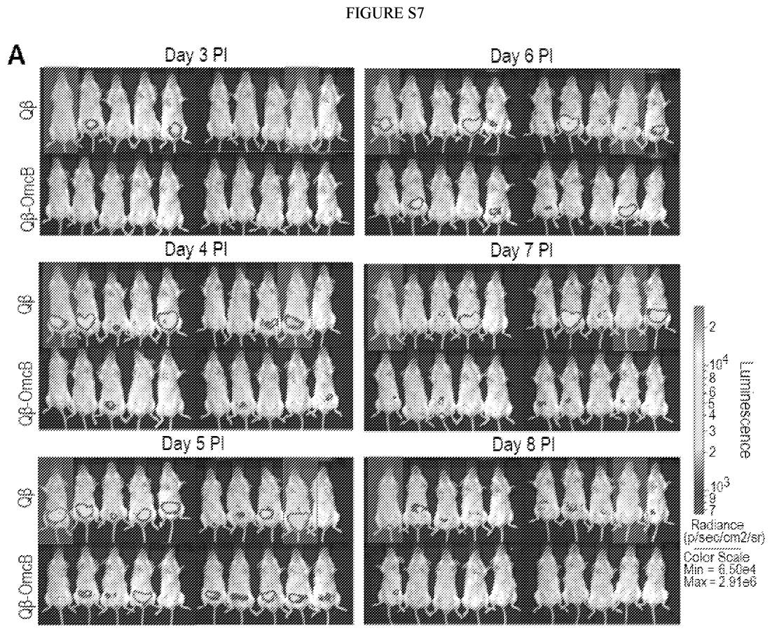

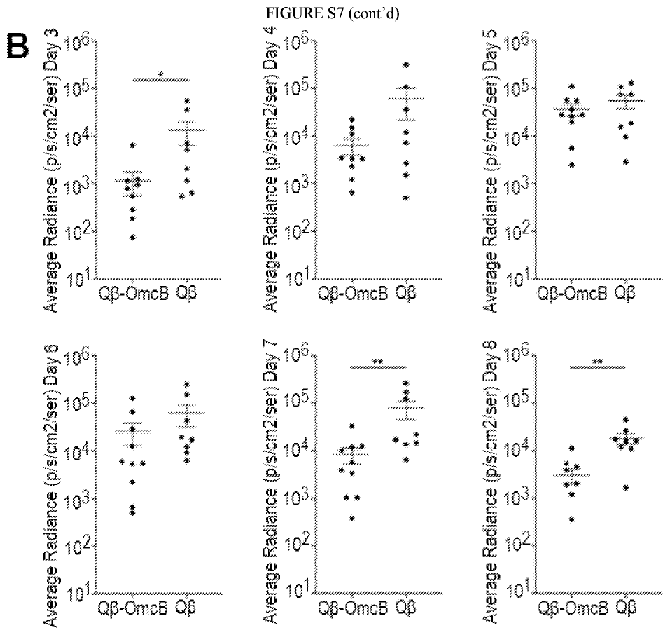

Having demonstrated that Qβ-Cm OmcB terminal immune sera IgG is able to bind to their cognate peptide and to both CtsvD and Cm elementary bodies, the inventors next investigated if immunization with Qβ-Cm OmcB is able to mediate protection against a vaginal Cm infection. Mice were immunized three times with Qβ-Cm OmcB or unconjugated Qβ and sera was collected after the final immunization to determine antibody titers (FIG. 11A). We found that immunization with Qβ-Cm OmcB elicited high-titer IgG antibodies that recognize the cognate peptide above that of the control (FIGURE S6). Vaccinated mice were challenged with 2×10⁴ IFU of Luciferase-expressing Cm instilled vaginally[30] and infection was monitored days 3 through 8 post-infection using IVIS (FIG. 11A). We demonstrate that bacterial burden is significantly lower on day 3 post-infection in Qβ-Cm OmcB vaccinated mice compared to controls, with a continued trend at day 4 post-infection (FIG. 11B, FIGURE S6). As infection continues, bacterial burden becomes similar between groups at days 5 and 6, but again decreases in Qβ-Cm OmcB immunized mice by days 7 and 8 (FIG. 11B, FIGURE S6). Indeed, by day 8 post-infection the Qβ-Cm OmcB vaccinated mice have little detectble infection via IVIS image (FIGURE S7). By measuring area under the curve, we demonstrate that, together, vaccination with Qβ-Cm OmcB results in a 0.56 log reduction in mean bacterial burden, which is a 72.5% reduction (FIG. 11C-D).

### Discussion

Urogenital Chlamydia trachomatis infection remains a major public health issue, for which screening programs alone have been unable to curb increasing prevalence of infection. Further, a subset of women who experience Ct infection go on to experience serious medical complications, including pelvic inflammatory disease, ectopic pregnancy, and tubal factor infertility. This underscores the urgent need for a prophylactic Ct vaccine to reduce healthcare costs, prevalence of infection, and morbidity associated with infection. Yet, a remaining barrier to producing an efficacious Ct vaccine is an understanding of protective immune responses, particularly the role of antibodies, along with identifying protective B cell epitopes.

In previous work, the inventors identified the human B cell epitopes of 24 chlamydial antigens, including important adhesion factors like OmcB[20]. They found that, aside from antibody responses to the Major Outer Membrane Protein, OmcB was the second most immunodominant antigen included in our library among females with urogenital Chlamydia trachomatis infection[20]. This knowledge, paired with extensive literature suggesting the key role of OmcB in Ct immunopathogenesis and host cell entry, made OmcB an attractive vaccine target[23,24,31]. The inventors hypothesized that targeting the immunodominant B cell epitope of OmcB through a VLP-based vaccine approach might elicit high titer antibodies that mediate protection from urogenital infection. Human infection with Ct is unable to provide naturally acquired protection, as many women experience repeated Ct infections, even with the same Ct serovar[32,33]. However, the inventors hypothesized that vaccine-induced antibodies may be at higher titers than would be elicited during natural infection, and thereby, provide necessary protection.

In these experiments, the inventors demonstrate that vaccination with a bacteriophage Qβ virus-like particle-based vaccine targeting the immunodominant B cell epitope of OmcB is highly immunogenic. Both Qβ-Ct OmcB and Qβ-Cm OmcB terminal immune sera IgG are able to bind to their cognate peptide, measured via ELISA. Further, terminal immune sera IgG can bind to Chlamydia itself, as measured by EB ELISAs. Also demonstrated was the moderate vaccine efficacy in a vaginal murine challenge model, decreasing the mean bacterial burden by 0.56 log.

Interestingly, the inventors also found that Qβ-Cm OmcB immune sera IgG is able to bind to CtsvD at potentially higher absorbance values than Qβ-Ct OmcB immune sera IgG, despite amino acid sequence differences. In agreement, Qβ-Cm OmcB immune sera IgG is able to bind to the Ct OmcB peptide. Further, Qβ-Cm OmcB immune sera IgG bound just as well, if not better, than the Qβ-VD4 MOMP positive control sera IgG to EBs, despite MOMP being much more prevalent on the surface of Ct than OmcB. This may signify antibody promiscuity, in which mature antibodies can have polyspecificity despite their rigid binding

pockets[34]. Additionally, Qβ-Cm OmcB immune sera may have higher avidity indexes, accounting for their higher absorbance values in the CtsvD EB ELISAs. Further investigation is needed to determine the quality of binding and Ct epitopes to which Qβ-Cm OmcB immune sera is able to bind.

Here, it was illustrated moderate vaccine efficacy, with a 0.56 log reduction in mean bacterial burden. This suggests that the immunodominant B cell epitope previously identified in our lab may be an important epitope in Ct entry into host cells. Yet, this epitope is likely not the only OmcB epitope involved in host cell binding, as previous research found that polyclonal sera can reduce in vitro infectivity by 36-39% and recombinant OmcB protein added to an in vitro Ct infection can inhibit 70% of Ct infectivity[231. Therefore, further research is undertaken to show the efficacy of additional protective epitopes.

The moderate protection the inventors observed with the Qβ-Cm OmcB vaccine in the murine urogenital Cm challenge model suggests that additional research is warranted for this vaccine. Further research into vaccine efficacy is undertaken by utilizing additional relevant challenge models, including a vaginal Ct model. This is an important next step, as Ct is the human pathogen we aim to protect against. Further, vaccine efficacy may be further increased in this model. One advantage to utilizing the bacteriophage Qβ virus-like particle vaccine platform is the ease of engineering mixed vaccinations. Therefore, future research will focus on mixed vaccinations targeting multiple adhesion factors antigens, including OmcB, which may further reduce bacterial burden. Finally, since there is a well-established role for CD4⁺ T cells in protection, our vaccine could also be combined with vaccines that provide CD4⁺ T cell Ct immunity.

In the present examples, the inventors demonstrated antibody-mediated vaccine efficacy in a pre-clinical Chlamydia murine model using a novel bacteriophage Qβ VLP vaccine platform, evidencing the potential for this vaccine to provide pan-serovar protection with binding to all Ct serovars, and demonstrate a long-lasting immune response in the serum and urogenital tract of mice.

### References (First Set MOMP)

1. Bautista, C. T., Hollingsworth, B. P. & Sanchez, J. L. Repeat Chlamydia Diagnoses Increase the Hazard of Pelvic Inflammatory Disease among US Army Women: A Retrospective Cohort Analysis. Sexually Transmitted Diseases 45, 770-773 (2018).
2. Beatty, W. L., Byrne, G. I. & Morrison, R. P. Repeated and persistent infection with Chlamydia and the development of chronic inflammation and disease. Trends in Microbiology 2, 94-98 (1994).
3. Herzog, S. A. et al. Timing of progression from Chlamydia trachomatis infection to pelvic inflammatory disease: a mathematical modelling study. BMC Infectious Diseases 12, 187 (2012).
4. Zhong, G., Brunham, R. C., de la Maza, L. M., Darville, T. & Deal, C. National Institute of Allergy and Infectious Diseases workshop report: “Chlamydia vaccines: The way forward”. Vaccine 37, 7346-7354 (2019).
5. Zhu, H., Shen, Z., Luo, H., Zhang, W. & Zhu, X. Chlamydia trachomatis Infection-Associated Risk of Cervical Cancer. Medicine (Baltimore) 95, e3077 (2016).
6. Paavonen, J. & Eggert-Kruse, E. Chlamydia trachomatis: impact on human reproduction. Human Reproduction Update 5,433-447 (1999).
7. Wasserheit, J. N. Epidemiological synergy: Interrelationships between human immunodeficiency virus infection and other sexually transmitted diseases. Sex Transm Dis 19, 61-77 (1992).
8. Johnson, L. F. & Lewis, D. A. The Effect of Genital Tract Infections on HIV-1 Shedding in the Genital Tract: A Systematic Review and Meta-Analysis. Sexually Transmitted Diseases 35, 946-959 (2008).
9. Fleming, D. T. & Wasserheit, J. N. From epidemiological synergy to public health policy and practice: the contribution of other sexually transmitted diseases to sexual transmission of HIV infection. Sex Transm Infect 75,3-17 (1999).
10. Tao, G., Hoover, K. W., Leichliter, J. S., Peterman, T. A. & Kent, C. K. Self-Reported Chlamydia Testing Rates of Sexually Active Women Aged 15-25 Years in the United States, 2006-2008. Sexually Transmitted Diseases 39, 605-607 (2012).
11. Chlamydia Screening Percentages Reported by Commercial and Medicaid Plans by State and Year. https://www.cdc.gov/std/chlamydia/chlamydia-screening-2014.htm (2019).
12. Broutet, N., Fruth, U., Deal, C., Gottlieb, S. L. & Rees, H. Vaccines against sexually transmitted infections: The way forward. Vaccine 32, 1630-1637 (2014).
13. Rose, S. B., Garrett, S. M., Stanley, J. & Pullon, S. R. H. Chlamydia trachomatis and Neisseria gonorrhoeae Retesting and Reinfection Rates in New Zealand Health Care Settings: Implications for Sexually Transmitted Infection Control. Sexually Transmitted Diseases 47, 151-157 (2020).
14. Batteiger, B. E. et al. Repeated Chlamydia trachomatis Genital Infections in Adolescent Women. J Infect Dis 201, 42-51 (2010).
15. Geisler, W. M., Lensing, S. Y., Press, C. G. & Hook, E. W. Spontaneous Resolution of Genital Chlamydia trachomatis Infection in Women and Protection from Reinfection. J Infect Dis 207, 1850-1856 (2013).
16. Morrison, S. G., Su, H., Caldwell, H. D. & Morrison, R. P. Immunity to Murine Chlamydia trachomatis Genital Tract Reinfection Involves B Cells and CD4+ T Cells but Not CD8+ T Cells. Infect Immun 68, 6979-6987 (2000).
17. Murthy, A. K. et al. Tumor Necrosis Factor Alpha Production from CD8+ T Cells Mediates Oviduct Pathological Sequelae following Primary Genital Chlamydia muridarum Infection. Infection and Immunity (2011).
18. Vlcek, K. R. et al. The Contribution of Chlamydia-Specific CD8+ T Cells to Upper Genital Tract Pathology. Immunol Cell Biol 94, 208-212 (2016).
19. Lijek, R. S., Helble, J. D., Olive, A. J., Seiger, K. W. & Starnbach, M. N. Pathology after Chlamydia trachomatis infection is driven by nonprotective immune cells that are distinct from protective populations. Proc Natl Acad Sci USA 115, 2216-2221 (2018).
20. Johansson, M., Schon, K., Ward, M. & Lycke, N. Genital tract infection with Chlamydia trachomatis fails to induce protective immunity in gamma interferon receptor-deficient mice despite a strong local immunoglobulin A response. Infect Immun 65, 1032-1044 (1997).
21. Wang, S., Fan, Y., C.Brunham, R. C. & Yang, X. IFN-γ knockout mice show Th2-associated delayed-type hypersensitivity and the inflammatory cells fail to localize and control chlamydial infection. European Journal of Immunology 29, 3782-3792 (1999).
22. Poston, T. B. et al. Cervical Cytokines Associated With Chlamydia trachomatis Susceptibility and Protection. The Journal of Infectious Diseases 220, 330-339 (2019).
23. Chronic chlamydial genital infection in congenitally athymic nude mice. https://journals.asm.org/doi/epdf/10.1128/iai.48.3.847-849.1985 doi:10.1128/iai.48.3.847-849.1985.
24. Gondek, D. C., Olive, A. J., Stary, G. & Starnbach, M. N. CD4+ T cells are necessary and sufficient to confer protection against C. trachomatis infection in the murine upper genital tract. J Immunol 189, 2441-2449 (2012).
25. Morrison, R. P., Feilzer, K. & Tumas, D. B. Gene knockout mice establish a primary protective role for major histocompatibility complex class II-restricted responses in Chlamydia trachomatis genital tract infection. Infect Immun 63, 4661-4668 (1995).
26. Ness, R. B. et al. Chlamydia Antibodies, Chlamydia Heat Shock Protein, and Adverse Sequelae After Pelvic Inflammatory Disease: The PID Evaluation and Clinical Health (PEACH) Study. Sexually Transmitted Diseases 35, 129-135 (2008).
27. Tan, C. et al. Chlamydia trachomatis-Infected Patients Display Variable Antibody Profiles against the Nine-Member Polymorphic Membrane Protein Family. Infect Immun 77, 3218-3226 (2009).
28. Taylor, B. D. et al. The Role of Chlamydia trachomatis Polymorphic Membrane Proteins in Inflammation and Sequelae among Women with Pelvic Inflammatory Disease. Infect Dis Obstet Gynecol 2011, 989762 (2011).
29. Ramsey, K. H., Soderberg, L. S. & Rank, R. G. Resolution of chlamydial genital infection in B-cell-deficient mice and immunity to reinfection. Infect Immun 56, 1320-1325 (1988).
30. Su, H., Feilzer, K., Caldwell, H. D. & Morrison, R. P. Chlamydia trachomatis genital tract infection of antibody-deficient gene knockout mice. Infect Immun 65, 1993-1999 (1997).
31. Crane, D. D. et al. Chlamydia trachomatis polymorphic membrane protein D is a species-common pan-neutralizing antigen. Proc Natl Acad Sci USA 103, 1894-1899 (2006).
32. Olsen, A. W., Lorenzen, E. K., Rosenkrands, I., Follmann, F. & Andersen, P. Protective Effect of Vaccine Promoted Neutralizing Antibodies against the Intracellular Pathogen Chlamydia trachomatis. Front Immunol 8, 1652 (2017).
33. Abraham, S. et al. Safety and immunogenicity of the Chlamydia vaccine candidate CTH522 adjuvanted with CAF01 liposomes or aluminium hydroxide: a first-in-human, randomised, double-blind, placebo-controlled, phase 1 trial. The Lancet Infectious Diseases 19, 1091-1100 (2019).
34. Peterson, E. M., Cheng, X., Markoff, B. A., Fielder, T. J. & de la Maza, L. M. Functional and structural mapping of Chlamydia trachomatis species-specific major outer membrane protein epitopes by use of neutralizing monoclonal antibodies. Infect Immun 59, 4147-4153 (1991).
35. Verma, R. et al. The Chlamydia M278 Major Outer Membrane Peptide Encapsulated in the Poly(lactic acid)-Poly(ethylene glycol) Nanoparticulate Self-Adjuvanting Delivery System Protects Mice Against a Chlamydia muridarum Genital Tract Challenge by Stimulating Robust Systemic and Local Mucosal Immune Responses. Frontiers in Immunology 9, (2018).
36. Byrne, G. I. et al. Workshop on in vitro Neutralization of Chlamydia trachomatis: Summary of Proceedings. The Journal of Infectious Diseases 168, 415-420 (1993).
37. Peeling, R., Maclean, I. W. & Brunham, R. C. In vitro neutralization of Chlamydia trachomatis with monoclonal antibody to an epitope on the major outer membrane protein. Infect Immun 46, 484-488 (1984).
38. Naglak, E. K., Morrison, S. G. & Morrison, R. P. Neutrophils Are Central to Antibody-Mediated Protection against Genital Chlamydia. Infect Immun 85, e00409-17 (2017).
39. Kari, L. et al. Antibody Signature of Spontaneous Clearance of Chlamydia trachomatis Ocular Infection and Partial Resistance against Re-challenge in a Nonhuman Primate Trachoma Model. PLoS Negl Trop Dis 7, e2248 (2013).
40. Su, H., Zhang, Y. X., Barrera, 0., Watkins, N. G. & Caldwell, H. D. Differential effect of trypsin on infectivity of Chlamydia trachomatis: loss of infectivity requires cleavage of major outer membrane protein variable domains II and IV. Infect Immun 56, 2094-2100 (1988).
41. Su, H., Watkins, N. G., Zhang, Y. X. & Caldwell, H. D. Chlamydia trachomatis-host cell interactions: role of the chlamydial major outer membrane protein as an adhesin. Infect Immun 58, 1017-1025 (1990).
42. Swanson, A. F., Ezekowitz, R. A. B., Lee, A. & Kuo, C. Human Mannose-Binding Protein Inhibits Infection of HeLa Cells by Chlamydia trachomatis. Infect Immun 66, 1607-1612 (1998).
43. Puolakkainen, M., Kuo, C.-C. & Campbell, L. A. Chlamydia pneumoniae Uses the Mannose 6-Phosphate/Insulin-Like Growth Factor 2 Receptor for Infection of Endothelial Cells. Infect Immun 73, 4620-4625 (2005).
44. Collar, A. L. et al. Antibodies to Variable Domain 4 Linear Epitopes of the Chlamydia trachomatis Major Outer Membrane Protein Are Not Associated with Chlamydia Resolution or Reinfection in Women. mSphere 5, e00654-20 (2020).
45. Molina, D. M. et al. Identification of immunodominant antigens of Chlamydia trachomatis using proteome microarrays. Vaccine 28, 3014-3024 (2010).
46. Rahman, K. S. et al. Discovery of Human-Specific Immunodominant Chlamydia trachomatis B Cell Epitopes. mSphere 3, e00246-18 (2018).
47. Hepler, R. W. et al. Spectroscopic analysis of chlamydial major outer membrane protein in support of structure elucidation. Protein Sci 27, 1923-1941 (2018).
48. Østergaard, O. et al. Quantitative Protein Profiling of Chlamydia trachomatis Growth Forms Reveals Defense Strategies Against Tryptophan Starvation. Mol Cell Proteomics 15, 3540-3550 (2016).
49. Boje, S. et al. A multi-subunit Chlamydia vaccine inducing neutralizing antibodies and strong IFN-γ+ CMI responses protects against a genital infection in minipigs. Immunol Cell Biol 94, 185-195 (2016).
50. Motin, V. L., de la Maza, L. M. & Peterson, E. M. Immunization with a Peptide Corresponding to Chlamydial Heat Shock Protein 60 Increases the Humoral Immune Response in C3H Mice to a Peptide Representing Variable Domain 4 of the Major Outer Membrane Protein of Chlamydia trachomatis. Clin Diagn Lab Immunol 6, 356-363 (1999).
51. Peterson, E. M., Cheng, X., Qu, Z. & de la Maza, L. M. The effect of orientation within a chimeric peptide on the immunogenicity of Chlamydia trachomatis epitopes. Mol Immunol 33, 335-339 (1996).
52. Cheng, X., Pal, S., de la Maza, L. M. & Peterson, E. M. Characterization of the humoral response induced by a peptide corresponding to variable domain IV of the major outer membrane protein of Chlamydia trachomatis serovar E. Infect Immun 60, 3428-3432 (1992).
53. Jelinková, L. et al. An epitope-based malaria vaccine targeting the junctional region of circumsporozoite protein. NPJ Vaccines 6, 13 (2021).
54. Mogus, A. T. et al. Virus-Like Particle Based Vaccines Elicit Neutralizing Antibodies against the HIV-1 Fusion Peptide. Vaccines (Basel) 8, 765 (2020).
55. Webster, E. et al. Immunogenicity and Protective Capacity of a Virus-like Particle Vaccine against Chlamydia trachomatis Type 3 Secretion System Tip Protein, CT584. Vaccines (Basel) 10, 111 (2022).
56. Chackerian, B., Rangel, M., Hunter, Z. & Peabody, D. S. Virus and virus-like particle-based immunogens for Alzheimer's disease induce antibody responses against amyloid-β without concomitant T cell responses. Vaccine 24, 6321-6331 (2006).
57. Barron, A. L., White, H. J., Rank, R. G., Soloff, B. L. & Moses, E. B. A New Animal Model for the Study of Chlamydia trachomatis Genital Infections: Infection of Mice with the Agent of Mouse Pneumonitis. The Journal of Infectious Diseases 143, 63-66 (1981).
58. Phillips, D. M. & Burillo, C. A. Ultrastructure of the murine cervix following infection with Chlamydia trachomatis. Tissue and Cell 30, 446-452 (1998).
59. Hay, P. E. et al. Chlamydia trachomatis in women: the more you look, the more you find. Genitourin Med 70, 97-100 (1994).
60. Oriel, J. D. et al. Infection of the Uterine Cervix with Chlamydia trachomatis. The Journal of Infectious Diseases 137, 443-451 (1978).
61. Campbell, J. et al. Bioluminescence Imaging of Chlamydia muridarum Ascending Infection in Mice. PLoS One 9, e101634 (2014).
62. Phillips, S., Quigley, B. L. & Timms, P. Seventy Years of Chlamydia Vaccine Research — Limitations of the Past and Directions for the Future. Frontiers in Microbiology 10, (2019).
63. Detailed STD Facts—Chlamydia. https://www.cdc.gov/std/chlamydia/stdfact-chlamydia-detailed.htm (2022).
64. Gallichan, W. S. & Rosenthal, K. L. Effects of the Estrous Cycle on Local Humoral Immune Responses and Protection of Intranasally Immunized Female Mice against Herpes Simplex Virus Type 2 Infection in the Genital Tract. Virology 224, 487-497 (1996).
65. Venkatesh, K. K. & Cu-Uvin, S. Anatomic and Hormonal Changes in the Female Reproductive Tract Immune Environment during the Life Cycle: Implications for HIV/STI Prevention Research. American Journal of Reproductive Immunology 71, 495-504 (2014).
66. Callan, T., Woodcock, S. & Huston, W. M. Ascension of Chlamydia is moderated by uterine peristalsis and the neutrophil response to infection. PLoS Comput Biol 17, e1009365 (2021).
67. Albritton, H. L. et al. A novel whole-bacterial enzyme linked-immunosorbant assay to quantify Chlamydia trachomatis specific antibodies reveals distinct differences between systemic and genital compartments. PLoS ONE 12, e0183101 (2017).
68. Yang, C. et al. Chlamydia trachomatis Plasmid Gene Protein 3 Is Essential for the Establishment of Persistent Infection and Associated Immunopathology. mBio (2020) doi:10.1128/mBio.01902-20.

### References (Second Set OcmB)

1. U.S. Department of Health & Human Services, Centers for Disease Control and Prevention National Overview—Sexually Transmitted Disease Surveillance, 2019 Available online: https://www.cdc.gov/std/statistics/2019/overview.htm (accessed on 1 December 2021).
2. Tao, G.; Hoover, K.W.; Leichliter, J.S.; Peterman, T.A.; Kent, C.K. Self-Reported Chlamydia Testing Rates of Sexually Active Women Aged 15-25 Years in the United States, 2006-2008. Sex. Transm. Dis. 2012, 39, 605-607, doi:10.1097/OLQ.0b013e318254c837.
3. Chlamydia Screening Percentages Reported by Commercial and Medicaid Plans by State and Year Available online: https://www.cdc.gov/std/chlamydia/chlamydia-screening-2014.htm (accessed on 31 January 2022).
4. Johnson, L.F.; Lewis, D.A. The Effect of Genital Tract Infections on HIV-1 Shedding in the Genital Tract: A Systematic Review and Meta-Analysis. Sex. Transm. Dis. 2008, 35, 946-959, doi:10.1097/OLQ.0b013e3181812d15.
5. Wasserheit, J.N. Epidemiological Synergy: Interrelationships between Human Immunodeficiency Virus Infection and Other Sexually Transmitted Diseases. Sex. Transm. Dis. 1992, 19, 61-77.
6. Murthy, A.K.; Li, W.; Chaganty, B.K.R.; Kamalakaran, S.; Guentzel, M.N.; Seshu, J.; Forsthuber, T.G.; Zhong, G.; Arulanandam, B.P. Tumor Necrosis Factor Alpha Production from CD8+ T Cells Mediates Oviduct Pathological Sequelae Following Primary Genital Chlamydia muridarum Infection. Infect. Immun. 2011.
7. Wang, S.; Fan, Y.; C.Brunham, R.C.; Yang, X. IFN-γKnockout Mice Show Th2-Associated Delayed-Type Hypersensitivity and the Inflammatory Cells Fail to Localize and Control Chlamydial Infection. Eur. J. Immunol. 1999, 29, 3782-3792, doi:10.1002/(SICI)1521-4141(199911)29:11<3782::AID-IMMU3782>3.0.00;2-B.
8. Johansson, M.; Schon, K.; Ward, M.; Lycke, N. Genital Tract Infection with Chlamydia trachomatis Fails to Induce Protective Immunity in Gamma Interferon Receptor-Deficient Mice despite a Strong Local Immunoglobulin A Response. Infect. Immun. 1997, 65, 1032-1044.
9. Morrison, S.G.; Su, H.; Caldwell, H.D.; Morrison, R.P. Immunity to Murine Chlamydia trachomatis Genital Tract Reinfection Involves B Cells and CD4+ T Cells but Not CD8+T Cells. Infect. Immun. 2000, 68, 6979-6987.
10. Morrison, R.P.; Feilzer, K.; Tumas, D.B. Gene Knockout Mice Establish a Primary Protective Role for Major Histocompatibility Complex Class II-Restricted Responses in Chlamydia trachomatis Genital Tract Infection. Infect. Immun. 1995, 63, 4661-4668.
11. Gondek, D.C.; Olive, A.J.; Stary, G.; Starnbach, M.N. CD4+ T Cells Are Necessary and Sufficient to Confer Protection against C. Trachomatis Infection in the Murine Upper Genital Tract. J. Immunol. Baltim. Md 1950 2012, 189, 2441-2449, doi:10.4049/jimmuno1.1103032.
12. Taylor, B.D.; Darville, T.; Tan, C.; Bavoil, P.M.; Ness, R.B.; Haggerty, C.L. The Role of Chlamydia Trachomatis Polymorphic Membrane Proteins in Inflammation and Sequelae among Women with Pelvic Inflammatory Disease. Infect. Dis. Obstet. Gynecol. 2011, 2011, 989762, doi:10.1155/2011/989762.
13. Budrys, N.M.; Gong, S.; Rodgers, A.K.; Wang, J.; Louden, C.; Shain, R.; Schenken, R.S.; Zhong, G. Chlamydia trachomatis Antigens Recognized by Women With Tubal Factor Infertility, Normal Fertility, and Acute Infection. Obstet. Gynecol. 2012, 119, 1009-1016, doi:10.1097/A0G.0b013e3182519326.
14. Morrison, S.G.; Morrison, R.P. Resolution of Secondary Chlamydia trachomatis Genital Tract Infection in Immune Mice with Depletion of Both CD4+ and CD8+ T Cells. Infect. Immun. 2001, 69, 2643-2649, doi:10.1128/IAI.69.4.2643-2649.2001.
15. Olsen, A.W.; Lorenzen, E.K.; Rosenkrands, I.; Follmann, F.; Andersen, P. Protectie Effect of Vaccine Promoted Neutralizing Antibodies against the Intracellular Pathogen Chlamydia trachomatis. Front. Immunol. 2017, 8, 1652, doi:10.3389/fimmu.2017.01652.
16. Crane, D.D.; Carlson, J.H.; Fischer, E.R.; Bavoil, P.; Hsia, R.; Tan, C.; Kuo, C.; Caldwell, H.D. Chlamydia trachomatis Polymorphic Membrane Protein D Is a Species-Common Pan-Neutralizing Antigen. Proc. Natl. Acad. Sci. U S. A. 2006, 103, 1894-1899, doi:10.1073/pnas.0508983103.
17. Peterson, E.M.; Cheng, X.; Markoff, B.A.; Fielder, T.J.; de la Maza, L.M. Functional and Structural Mapping of Chlamydia trachomatis Species-Specific Major Outer Membrane Protein Epitopes by Use of Neutralizing Monoclonal Antibodies. Infect. Immun. 1991, 59, 4147-4153.
18. Boje, S.; Olsen, A.W.; Erneholm, K.; Agerholm, J.S.; Jungersen, G.; Andersen, P.; Follmann, F. A Multi-Subunit Chlamydia Vaccine Inducing Neutralizing Antibodies and Strong IFN-Γ+ CMI Responses Protects against a Genital Infection in Minipigs. Immunol. Cell Biol. 2016, 94, 185-195, doi:10.1038/icb.2015.79.
19. Gupta, K.; Brown, L.; Bakshi, R.K.; Press, C.G.; Chi, X.; Gorwitz, R.J.; Papp, J.R.; Geisler, W.M. Performance of Chlamydia trachomatis OmcB Enzyme-Linked Immunosorbent Assay in Serodiagnosis of Chlamydia trachomatis Infection in Women. J. Clin. Microbiol. 2018, 56, e00275-18, doi:10.1128/JCM.00275-18.
20. Collar, A.L.; Linville, A.C.; Core, S.B.; Wheeler, C.M.; Geisler, W.M.; Peabody, D.S.; Chackerian, B.; Frietze, K.M. Antibodies to Variable Domain 4 Linear Epitopes of the Chlamydia trachomatis Major Outer Membrane Protein Are Not Associated with Chlamydia Resolution or Reinfection in Women. mSphere 2020, 5, e00654-20, doi:10.1128/mSphere.00654-20.
21. Mygind, P.; Christiansen, G.; Birkelund, S. Topological Analysis of Chlamydia trachomatis L2 Outer Membrane Protein 2. J. Bacteriol. 1998, 180, 5784-5787.
22. Newhall, W.J. Biosynthesis and Disulfide Cross-Linking of Outer Membrane Components during the Growth Cycle of Chlamydia trachomatis. Infect. Immun. 1987, 55, 162-168.
23. Fadel, S.; Eley, A. Chlamydia trachomatis OmcB Protein Is a Surface-Exposed Glycosaminoglycan-Dependent Adhesin. J. Med. Microbiol. 2007, 56, 15-22, doi:10.1099/jmm.0.46801-0.
24. Fechtner, T.; Stallmann, S.; Moelleken, K.; Meyer, K.L.; Hegemann, J.H. Characterization of the Interaction between the Chlamydial Adhesin OmcB and the Human Host Cell. J. Bacteriol. 2013, 195, 5323-5333, doi:10.1128/JB.00780-13.
25. Warner, N.L.; Frietze, K.M. Development of Bacteriophage Virus-Like Particle Vaccines Displaying Conserved Epitopes of Dengue Virus Non-Structural Protein 1. Vaccines 2021, 9, 726, doi:10.3390/vaccines9070726.
26. Crossey, E.; Amar, M.J.A.; Sampson, M.; Peabody, J.; Schiller, J.T.; Chackerian, B.; Remaley, A.T. A Cholesterol-Lowering VLP Vaccine That Targets PCSK9. Vaccine 2015, 33, 5747-5755, doi:10.1016/j.vaccine.2015.09.044.
27. Webster, E.; Seiger, K.W.; Core, S.B.; Collar, A.L.; Knapp-Broas, H.; Graham, J.; Shrestha, M.; Afzaal, S.; Geisler, W.M.; Wheeler, C.M.; et al. Immunogenicity and Protective Capacity of a Virus-like Particle Vaccine against Chlamydia trachomatis Type 3 Secretion System Tip Protein, CT584. Vaccines 2022, 10, 111, doi:10.3390/vaccines10010111.
28. Lijek, R.S.; Helble, J.D.; Olive, A.J.; Seiger, K.W.; Starnbach, M.N. Pathology after Chlamydia trachomatis Infection Is Driven by Nonprotective Immune Cells That Are Distinct from Protective Populations. Proc. Natl. Acad. Sci. U S. A. 2018, 115, 2216-2221, doi:10.1073/pnas.1711356115.
29. Albritton, H.L.; Kozlowski, P.A.; Lillis, R.A.; McGowin, C.L.; Siren, J.D.; Taylor, S.N.; Ibana, J.A.; Buckner, L.R.; Shen, L.; Quayle, A.J. A Novel Whole-Bacterial Enzyme Linked-Immunosorbant Assay to Quantify Chlamydia trachomatis Specific Antibodies Reveals Distinct Differences between Systemic and Genital Compartments. PLOS ONE 2017, 12, e0183101, doi:10.1371/journal.pone.0183101.
30. Campbell, J.; Huang, Y.; Liu, Y.; Schenken, R.; Arulanandam, B.; Zhong, G. Bioluminescence Imaging of Chlamydia muridarum Ascending Infection in Mice. PLoS ONE 2014, 9, e101634, doi:10.1371/journal.pone.0101634.
31. Moelleken, K.; Hegemann, J.H. The Chlamydia Outer Membrane Protein OmcB Is Required for Adhesion and Exhibits Biovar-Specific Differences in Glycosaminoglycan Binding. Mol. Microbiol. 2008, 67, 403-419, doi:10.1111/j.1365-2958.2007.06050.x.
32. Rose, S.B.; Garrett, S.M.; Stanley, J.; Pullon, S.R.H. Chlamydia trachomatis and Neisseria Gonorrhoeae Retesting and Reinfection Rates in New Zealand Health Care Settings: Implications for Sexually Transmitted Infection Control. Sex. Transm. Dis. 2020, 47, 151-157, doi:10.1097/OLQ.0000000000001112.
33. Batteiger, B.E.; Tu, W.; Other, S.; Van Der Pol, B.; Stothard, D.R.; Orr, D.P.; Katz, B.P.; Fortenberry, J.D. Repeated Chlamydia trachomatis Genital Infections in Adolescent Women. J. Infect. Dis. 2010, 201, 42-51, doi:10.1086/648734.
34. Kaur, H.; Salunke, D.M. Antibody Promiscuity: Understanding the Paradigm Shift in Antigen Recognition. IUBMB Life 2015, 67, 498-505, doi:10.1002/iub.1397.

| Sequences of ImmunoPeptides | SEQ ID NO: |
|---|---|
| FEGNSANFANGGAIAASGKVLFVAN | SEQ ID NO: 1 |
| GAQLTQTTSGSS | SEQ ID NO: 2 |
| ARAPQAIPTRSSD | SEQ ID NO: 3 |
| DLEDSVNSEK | SEQ ID NO: 4 |
| DGYKGAYGGASA | SEQ ID NO: 5 |
| TFFLEEEKLPSEAF | SEQ ID NO: 6 |
| SEQENNAEIG | SEQ ID NO: 7 |
| ALFASEDGDLSPESS | SEQ ID NO: 8 |
| STPVQQGHAISK | SEQ ID NO: 9 |
| QGHAISKPEAEIE | SEQ ID NO: 10 |
| SKPEAEIESSSEP | SEQ ID NO: 11 |
| ARAPQALPTQEEF | SEQ ID NO: 12 |
| KKEGRPLSSGYS | SEQ ID NO: 13 |
| ASEDGDLSPE | SEQ ID NO: 14 |
| GGAICTQNLTISQNTGNVLFYNN | SEQ ID NO: 15 |
| FEGNSAQLANGGAIAASGKVLFVAN | SEQ ID NO: 16 |
| GGAICTQNLTISQNNGNVLFLNN | SEQ ID NO: 17 |
| DGYRGSYIGASA | SEQ ID NO: 18 |
| LEEEKLPSE | SEQ ID NO: 19 |
| FASEDGDLSPESSISSEE | SEQ ID NO: 20 |
| FLEEEKLPSEAFISAEE | SEQ ID NO: 21 |
| GSQGDTADT | SEQ ID NO: 22 |
| APSGDQS | SEQ ID NO: 23 |
| LEGSQGDTADTGT | SEQ ID NO: 24 |
| FDTTTLNPTIAGAGDVK | SEQ ID NO: 25 |
| VDITTLNPTIAGSGSVA | SEQ ID NO: 26 |
| LDVTTLNPTIAGKGAVV | SEQ ID NO: 27 |
| LDVTTLNPTIAGKGTVV | SEQ ID NO: 28 |
| VDITTLNPTIAGSGSVV | SEQ ID NO: 29 |
| EFTINKPKGYVGKEFPLD | SEQ ID NO: 30 |
| LDVTTWNPTIAGAGTIA | SEQ ID NO: 31 |
| LKMTTWNPTISGSGI | SEQ ID NO: 32 |
| EFTINKPKGYVGQEFPLN | SEQ ID NO: 33 |
| ARKNHSK | SEQ ID NO: 34 |
| NTSHKSKKARKNSHSKET | SEQ ID NO: 35 |
| ATGPKQDSSFGRMY | SEQ ID NO: 36 |
| TTSHQKDRKARKNHQNRT | SEQ ID NO: 37 |
| ARKNHQN | SEQ ID NO: 38 |
| AVEPRQDSCFGKMY | SEQ ID NO: 39 |
| KSTPVAAKMTAS | SEQ ID NO: 40 |
| TNTGLTPTT | SEQ ID NO: 41 |
| SNNSSTNA | SEQ ID NO: 42 |
| RTSITNTGLT | SEQ ID NO: 43 |
| STTPVAAKITAS | SEQ ID NO: 44 |
| TNSGSTPTT | SEQ ID NO: 45 |
| TNNAQANS | SEQ ID NO: 46 |
| KTCVTNSGST | SEQ ID NO: 47 |
| SNNPSTNA | SEQ ID NO: 48 |
| INTGLTPTT | SEQ ID NO: 49 |
| GDSKPYAISYGY | SEQ ID NO: 50 |
| GDTQPCAISYGY | SEQ ID NO: 51 |
| TSTPVAAKMTAS | SEQ ID NO: 52 |
| RTRIINTGLT | SEQ ID NO: 53 |
| VDITTLNPTIAGCGSVV | SEQ ID NO: 54 |
| FDVTTLNPTIAGAGDVK | SEQ ID NO: 55 |
| LDVTTLNPTIAGKGSVV | SEQ ID NO: 56 |
| FDTTTLNPTIAGAGEVK | SEQ ID NO: 57 |
| AVEPRQDSCFGKMY | SEQ ID NO: 58 |
| ATGPKQDSCFGRMYGGGC | SEQ ID NO: 59 |
| TTLNPTIAG | SEQ ID NO: 60 |
| EDEQIGARIV | SEQ ID NO: 61 |
| NIYESIGGSRTSGPEN | SEQ ID NO: 62 |
| LNPTIAG | SEQ ID NO: 63 |
| LNPTIAGA | SEQ ID NO: 64 |
| APSGDQS | SEQ ID NO: 65 |
| PSGDQSI | SEQ ID NO: 66 |
| GSQGDTADT | SEQ ID NO: 67 |
| SQGDTADTG | SEQ ID NO: 68 |
| ASSGAPSGDQSISANA | SEQ ID NO: 69 |
| TNLEGSQGDTADTGTGDVN | SEQ ID NO: 70 |
| EFTINKPKGYVGKEFPLD | SEQ ID NO: 71 |
| FDTTTLNPTIAGAGDV | SEQ ID NO: 72 |
| ATGPKQDSCFGRMY | SEQ ID NO: 73 |
| LEGSQGDTADTGT | SEQ ID NO: 74 |
| ASEDGDLSPE | SEQ ID NO: 75 |

1. A composition comprising: (a) a virus-like particle (VLP) comprising a bacteriophage coat protein; and (b) at least one immunogenic peptide; wherein said peptide is displayed on said virus-like particle, and wherein said peptide comprises a conjugated antigenic determinant of at least five (5) contiguous amino acid residues of a peptide sequence according to SEQIDNOS:1-75.

2. The composition of claim 1, wherein said peptide conjugate is displayed at one or more lysine amino acid residues at the N-terminus or carboxy terminus of said bacteriophage coat protein.

3. The composition of claim 1, wherein said peptide conjugate is displayed at high density on the surface of said VLP.

4. The composition of claim 1, wherein the bacteriophage coat protein is a coat protein derived from Qbeta, MS2, or AP205 bacteriophage.

5. The composition of claim 1, wherein said bacteriophage coat protein is a coat protein derived from Qbeta bacteriophage.

6. (canceled)

7. The composition according to claim 1, wherein said peptide conjugate is displayed at one or more lysine residues on the surface of the VLP.

8. The composition according to claim 2 wherein said peptide conjugate is displayed on said bacteriophage at said lysine residues by covalently binding said immunogenic peptide to said lysine residues through a linker group.

9. The composition according to claim 8 wherein said linker group comprises an oligopeptide linker covalently bonded to a crosslinker.

10. The composition according to claim 9 wherein said oligopeptide linker is covalently bonded to an electrophilic or nucleophilic group on the immunogenic peptide and the crosslinker is bonded to said lysine residues on the surface of said bacteriophage.

11. The composition according to claim 9 wherein said oligopeptide is a 4 to 15 mer oligopeptide comprising neutral amino acid residues bonded to an amine group on said opioid molecule.

12. The composition according to claim 11 wherein said neutral amino acid residues are selected from the group consisting of glycine, alanine, valine, leucine, isoleucine, phenylalanine, tryptophan, methione, proline, serine and mixtures thereof.)

13. The composition according to claim 11 wherein said neutral amino acids are selected from the group consisting of glycine, serine and mixture thereof.

14. (canceled)

15. (canceled)

16. (canceled)

17. The composition according to claim 1 wherein said immunogenic peptide is a peptide sequence according to any one of SEQIDNOs 1-75.

18. (canceled)

19. (canceled)

20. (canceled)

21. (canceled)

22. (canceled)

23. (canceled)

24. (canceled)

25. (canceled)

26. The composition according to claim 1 wherein said immunogenic peptide is a peptide sequence according to any one of SEQIDNOs 25-29, 54-58 or 71-75.

27. (canceled)

28. (canceled)

29. (canceled)

30. A composition comprising: (a) a virus-like particle (VLP) comprising a bacteriophage coat protein; and (b) at least one immunogenic peptide; wherein said peptide is displayed on said virus-like particle, and wherein said peptide comprises a conjugated antigenic determinant of a peptide sequence according any one of SEQIDNOS:1-75, wherein said bacteriophage coat protein is a dimeric Qbeta coat protein which conjugates said immunogenic peptide to lysine residues on the surface of said VLP.

31. A population of virus-like particles according to claim 1.

32. A pharmaceutical composition comprising a population of virus-like particles according to claim 31 in combination with a pharmaceutically acceptable carrier, additive and/or excipient.

33. The composition according to claim 32 which is formulated as a vaccine for administration to a subject or patient.

34. The composition according to claim 33 wherein said vaccine comprises an adjuvant.

35. A method for enhancing an immune response against an immunogenic peptide in a patient or subject in need comprising introducing the composition of claim 32 into said subject or patient, wherein an enhanced immune response against said immunogenic peptide is produced in said patient or subject.

36. The method of claim 35, wherein the composition is prophylactic for a Ct infection.

37. A method of inducing an immunogenic response in a patient or subject in a patient or subject in need comprising administering to said patient or subject a composition according to claim 32 to said patient or subject.

38. A method for treating or inhibiting Chlamydia trachomatis (Ct) infection or a symptom or morbidity thereof in a patient or subject in need comprising administering to said patient a composition according to claim 32 to said patient or subject.

39. A method for treating or reducing the likelihood of a Chlamydia trachomatis (Ct) infection in a patient or subject in need comprising administering to said patient or subject a composition according to claim 32 to said patient or subject.


SEQUENCE LISTING (75 sequences)

SEQ ID NO: 1 - protein, 25 aa, Chlamydia trachomatis
FEGNSANFANGGAIAASGKVLFVAN

SEQ ID NO: 2 - protein, 12 aa, Chlamydia trachomatis
GAQLTQTTSGSS

SEQ ID NO: 3 - protein, 13 aa, Chlamydia trachomatis
ARAPQAIPTRSSD

SEQ ID NO: 4 - protein, 10 aa, Chlamydia trachomatis
DLEDSVNSEK

SEQ ID NO: 5 - protein, 12 aa, Chlamydia trachomatis
DGYKGAYGGASA

SEQ ID NO: 6 - protein, 14 aa, Chlamydia trachomatis
TFFLEEEKLPSEAF

SEQ ID NO: 7 - protein, 10 aa, Chlamydia trachomatis
SEQENNAEIG

SEQ ID NO: 8 - protein, 15 aa, Chlamydia trachomatis
ALFASEDGDLSPESS

SEQ ID NO: 9 - protein, 12 aa, Chlamydia trachomatis
STPVQQGHAISK

SEQ ID NO: 10 - protein, 13 aa, Chlamydia trachomatis
QGHAISKPEAEIE

SEQ ID NO: 11 - protein, 13 aa, Chlamydia trachomatis
SKPEAEIESSSEP

SEQ ID NO: 12 - protein, 13 aa, Chlamydia trachomatis
ARAPQALPTQEEF

SEQ ID NO: 13 - protein, 12 aa, Chlamydia trachomatis
KKEGRPLSSGYS

SEQ ID NO: 14 - protein, 10 aa, Chlamydia trachomatis
ASEDGDLSPE

SEQ ID NO: 15 - protein, 23 aa, Chlamydia trachomatis
GGAICTQNLTISQNTGNVLFYNN

SEQ ID NO: 16 - protein, 25 aa, Chlamydia trachomatis
FEGNSAQLANGGAIAASGKVLFVAN

SEQ ID NO: 17 - protein, 23 aa, Chlamydia trachomatis
GGAICTQNLTISQNNGNVLFLNN

SEQ ID NO: 18 - protein, 12 aa, Chlamydia trachomatis
DGYRGSYIGASA

SEQ ID NO: 19 - protein, 9 aa, Chlamydia trachomatis
LEEEKLPSE

SEQ ID NO: 20 - protein, 18 aa, Chlamydia trachomatis
FASEDGDLSPESSISSEE

SEQ ID NO: 21 - protein, 17 aa, Chlamydia trachomatis
FLEEEKLPSEAFISAEE

SEQ ID NO: 22 - protein, 9 aa, Chlamydia trachomatis
GSQGDTADT

SEQ ID NO: 23 - protein, 7 aa, Chlamydia trachomatis
APSGDQS

SEQ ID NO: 24 - protein, 13 aa, Chlamydia trachomatis
LEGSQGDTADTGT

SEQ ID NO: 25 - protein, 17 aa, Chlamydia trachomatis
FDTTTLNPTIAGAGDVK

SEQ ID NO: 26 - protein, 17 aa, Chlamydia trachomatis
VDITTLNPTIAGSGSVA

SEQ ID NO: 27 - protein, 17 aa, Chlamydia trachomatis
LDVTTLNPTIAGKGAVV

SEQ ID NO: 28 - protein, 17 aa, Chlamydia trachomatis
LDVTTLNPTIAGKGTVV

SEQ ID NO: 29 - protein, 17 aa, Chlamydia trachomatis
VDITTLNPTIAGSGSVV

SEQ ID NO: 30 - protein, 18 aa, Chlamydia trachomatis
EFTINKPKGYVGKEFPLD

SEQ ID NO: 31 - protein, 17 aa, Chlamydia trachomatis
LDVTTWNPTIAGAGTIA

SEQ ID NO: 32 - protein, 15 aa, Chlamydia trachomatis
LKMTTWNPTISGSGI

SEQ ID NO: 33 - protein, 18 aa, Chlamydia trachomatis
EFTINKPKGYVGQEFPLN

SEQ ID NO: 34 - protein, 7 aa, Chlamydia trachomatis
ARKNHSK

SEQ ID NO: 35 - protein, 18 aa, Chlamydia trachomatis
NTSHKSKKARKNSHSKET

SEQ ID NO: 36 - protein, 14 aa, Chlamydia trachomatis
ATGPKQDSSFGRMY

SEQ ID NO: 37 - protein, 18 aa, Chlamydia trachomatis
TTSHQKDRKARKNHQNRT

SEQ ID NO: 38 - protein, 7 aa, Chlamydia trachomatis
ARKNHQN

SEQ ID NO: 39 - protein, 14 aa, Chlamydia trachomatis
AVEPRQDSCFGKMY

SEQ ID NO: 40 - protein, 12 aa, Chlamydia trachomatis
KSTPVAAKMTAS

SEQ ID NO: 41 - protein, 9 aa, Chlamydia trachomatis
TNTGLTPTT

SEQ ID NO: 42 - protein, 8 aa, Chlamydia trachomatis
SNNSSTNA

SEQ ID NO: 43 - protein, 10 aa, Chlamydia trachomatis
RTSITNTGLT

SEQ ID NO: 44 - protein, 12 aa, Chlamydia trachomatis
STTPVAAKITAS

SEQ ID NO: 45 - protein, 9 aa, Chlamydia trachomatis
TNSGSTPTT

SEQ ID NO: 46 - protein, 8 aa, Chlamydia trachomatis
TNNAQANS

SEQ ID NO: 47 - protein, 10 aa, Chlamydia trachomatis
KTCVTNSGST

SEQ ID NO: 48 - protein, 8 aa, Chlamydia trachomatis
SNNPSTNA

SEQ ID NO: 49 - protein, 9 aa, Chlamydia trachomatis
INTGLTPTT

SEQ ID NO: 50 - protein, 12 aa, Chlamydia trachomatis
GDSKPYAISYGY

SEQ ID NO: 51 - protein, 12 aa, Chlamydia trachomatis
GDTQPCAISYGY

SEQ ID NO: 52 - protein, 12 aa, Chlamydia trachomatis
TSTPVAAKMTAS

SEQ ID NO: 53 - protein, 10 aa, Chlamydia trachomatis
RTRIINTGLT

SEQ ID NO: 54 - protein, 17 aa, Chlamydia trachomatis
VDITTLNPTIAGCGSVV

SEQ ID NO: 55 - protein, 17 aa, Chlamydia trachomatis
FDVTTLNPTIAGAGDVK

SEQ ID NO: 56 - protein, 17 aa, Chlamydia trachomatis
LDVTTLNPTIAGKGSVV

SEQ ID NO: 57 - protein, 17 aa, Chlamydia trachomatis
FDTTTLNPTIAGAGEVK

SEQ ID NO: 58 - protein, 14 aa, Chlamydia trachomatis
AVEPRQDSCFGKMY

SEQ ID NO: 59 - protein, 18 aa, Chlamydia trachomatis
ATGPKQDSCFGRMYGGGC

SEQ ID NO: 60 - protein, 9 aa, Chlamydia trachomatis
TTLNPTIAG

SEQ ID NO: 61 - protein, 10 aa, Chlamydia trachomatis
EDEQIGARIV

SEQ ID NO: 62 - protein, 16 aa, Chlamydia trachomatis
NIYESIGGSRTSGPEN

SEQ ID NO: 63 - protein, 7 aa, Chlamydia trachomatis
LNPTIAG

SEQ ID NO: 64 - protein, 8 aa, Chlamydia trachomatis
LNPTIAGA

SEQ ID NO: 65 - protein, 7 aa, Chlamydia trachomatis
APSGDQS

SEQ ID NO: 66 - protein, 7 aa, Chlamydia trachomatis
PSGDQSI

SEQ ID NO: 67 - protein, 9 aa, Chlamydia trachomatis
GSQGDTADT

SEQ ID NO: 68 - protein, 9 aa, Chlamydia trachomatis
SQGDTADTG

SEQ ID NO: 69 - protein, 16 aa, Chlamydia trachomatis
ASSGAPSGDQSISANA

SEQ ID NO: 70 - protein, 19 aa, Chlamydia trachomatis
TNLEGSQGDTADTGTGDVN

SEQ ID NO: 71 - protein, 18 aa, Chlamydia trachomatis
EFTINKPKGYVGKEFPLD

SEQ ID NO: 72 - protein, 16 aa, Chlamydia trachomatis
FDTTTLNPTIAGAGDV

SEQ ID NO: 73 - protein, 14 aa, Chlamydia trachomatis
ATGPKQDSCFGRMY

SEQ ID NO: 74 - protein, 13 aa, Chlamydia trachomatis
LEGSQGDTADTGT

SEQ ID NO: 75 - protein, 10 aa, Chlamydia trachomatis
ASEDGDLSPE